(19) United States
(12) Patent Application Publication
(10) Pub. No.: US 2015/0306187 A1
(43) Pub. Date: Oct. 29, 2015

(54) SUBCUTANEOUS ADMINISTRATION OF ALPHA-GALACTOSIDASE A

(71) Applicant: Shire Human Genetic Therapies, Inc., Lexington, MA (US)

(72) Inventors: Lisa Marie Sturk, Boston, MA (US); Justin C. Lamsa, Westminster, MA (US); Michael W. Heartlein, Boxborough, MA (US); Vinh Nguyen, Chelsea, MA (US); Katherine D. Taylor, Arlington, MA (US); Zahra Shahrokh, Weston, MA (US)

(21) Appl. No.: 14/702,154

(22) Filed: May 1, 2015

Related U.S. Application Data
(63) Continuation of application No. 13/902,297, filed on May 24, 2013, now abandoned.
Application No. 13/902,297 is a continuation of application No. 12/675,591, filed on Aug. 5, 2010, now abandoned.
(60) Provisional application No. 60/966,722, filed on Aug. 29, 2007.

(51) Int. Cl. A61K 38/47

(52) U.S. Cl. CPC A61K 38/47; C12Y 302/01022

(57) ABSTRACT

The invention relates, in part, to improved methods of administering α-galactosidase A for the treatment of α-galactosidase A deficiencies including Fabry disease.

### RELATED APPLICATIONS

This application is a continuation of U.S. application Ser. No. 13/902,297 filed May 24, 2013, now abandoned, which is a continuation of U.S. application Ser. No. 12/675,591 filed Aug. 5, 2010, which is a national stage filing under 35 U.S.C. §371 of international application PCT/US2008/010212, filed Aug. 28, 2008, which was published under PCT Article 21(2) in English, and claims benefit under 35 U.S.C. §119(e) of U.S. provisional application 60/966,722, filed Aug. 29, 2007, the disclosures of each of which are incorporated in their entirety herein by reference.

### FIELD OF THE INVENTION

This invention relates to improved methods of treating α-galactosidase A deficiencies, including Fabry disease, through the administration of α-galactosidase A compositions.

### BACKGROUND OF THE INVENTION

Fabry disease is an X-linked disorder characterized by the absence of a-galactosidase A (α-Gal A), an enzyme required for the normal processing of glycosphingolipids in mammalian lysosomes. The loss of α-Gal A leads to accumulation of the neutral globotriaosylceramide (Gb3), also known as ceramide trihexoside (CTH), within the heart, kidney, liver, and vascular endothelial cells. Renal and cardiac diseases are the most common cause of mortality and morbidity in Fabry patients (Thurberg et al., 2002 Kidney International, 62(6): 1933-1946; Tanaka et al., 2005 Clinical Nephrology, 64(4): 281-287). Hemizygous males, homozygous females, and some heterozygous females experience progressive organ dysfunction manifesting clinically as angiokeratomas, acroparathesis, stroke, cardiomyopathies, myocardian infarction and renal failure (Thurberg et al., 2002 Kidney International, 62(6): 1933-1946). The kidney is exceptionally susceptible to damage from Gb3 deposition with several published reports of glycosphingolipid localized to the podocytes, vascular endothelial cells, and epithelial cells of the glomerulus. Loss of podocytes by apoptosis leads to glomerulosclerosis and drastically reduced kidney function. Affected individuals vary in disease progression and severity of symptoms.

Historically, treatment options for Fabry patients were limited to symptomatic relief of renal and cardiovascular complications (Desnick et al., 2002 Clinical Nephrology, 57(1):1-8). Attempts at more severe treatments, namely organ transplantation (Cho and Kopp, 2004 Pediatr Nephrol, 19(6):583-593; Sessa et al., 2002 Nephron, 91(2): 348-351) and plasmapheresis (Winters et al., 2000 J Clin Apheresis, 15(1-2):53-73), did not prove successful. Currently, two galactosidase drugs are available for treatment of Fabry disease via enzyme replacement therapy (ERT): agalsidase alfa (Replagal®, TKT/Shire) and agalsidase beta (Fabrazyme®, Genzyme). These protein based therapeutics are administered by (approved for) intravenous injection and deliver galactosidase activity to the lysomomes of affected organs in order to reduce the level of Gb3 accumulation. Additional approaches to ERT for treatment of lysosomal storage diseases, such as Fabry disease, are needed.

### SUMMARY OF THE INVENTION

By understanding the pharmacokinetics and modification profile (e.g., carbohydrate, phosphate or sialylation modification) of human α-Gal A, we have developed novel pharmaceutical compositions of α-Gal A, kits for treatment of α-Gal A deficiency, methods of selecting an appropriate dose of α-Gal A for a patient, and methods of treating α-Gal A deficiency using such compositions. Also provided are methods of evaluating α-Gal A preparations, samples, batches, and the like, e.g., methods of quality control and determination of bioequivalence, e.g., with reference to the α-Gal A compositions described herein.

According to one aspect of the invention, methods of enhancing delivery of α-Gal A to the kidneys in an individual with Fabry disease are provided. The methods include administering human α-Gal A subcutaneously to the individual. In some embodiments, the α-Gal A is administered in a sufficient dose to result in a peak concentration of α-Gal A in the kidney of the subject within about 24 hours after the administration of the dose. In certain embodiments, the α-Gal A is administered in sufficient dose to result in a peak concentration of α-Gal A in the kidney of the subject within about 45, 40, 35, 30, 25, or fewer hours after the administration of the dose. In some embodiments, the dose does not result in a toxic level of α-Gal A in the liver of the individual. In some embodiments, the α-Gal A is administered in sufficient dose to result in kidney α-Gal A levels in the individual that result in an increase in the fraction of normal glomeruli and/or a decrease in the fraction of glomeruli with mesangial widening. In some embodiments, α-Gal A is isolated, genetically engineered α-Gal A. In certain embodiments, the genetically engineered α-Gal A is produced in a human cell, a yeast cell, a bacterial cell, an insect cell, or a plant cell. In some embodiments, the α-Gal A is administered in an α-Gal A formulation. In some embodiments, the formulation of the α-Gal A is a single dose formulation. In some embodiments, the formulation of the α-Gal A includes from about 1 mg/ml to about 60 mg/ml α-Gal A, from about 2% to about 10% (w/v) carbohydrate, from about 5 mM to about 10 mM citrate, up to 3% (v/v) excipient, and from about 0.05% to about 0.5% (v/v) surfactant. In certain embodiments, the carbohydrate is sucrose. In some embodiments, the pH of the formulation is about 6.0. In some embodiments, the excipient is glycerol. In some embodiments, the surfactant is poloxamer 188. In certain embodiments, the single dose formulation includes 30 mg/ml of α-Gal A, 5% (w/v) sucrose, 5 mM citrate, between about 1% and 2.5% (v/v) glycerol, and 0.05% (v/v) poloxamer 188, and wherein the pH of the formulation is 6.0. In some embodiments, the formulation of the α-Gal A is a multi-dose formulation. In some embodiments, the formulation of the α-Gal A includes from about 1 mg/ml to about 60 mg/ml α Gal A, from about 2% to about 10% (w/v) carbohydrate, from about 5 mM to about 10 mM citrate, about 1% or less of an antimicrobial agent, and up to 3% (v/v) excipient. In certain embodiments, the pH of the formulation is about 6.0. In some embodiments, the carbohydrate is sucrose. In some embodiments, the excipient is glycerol. In certain embodiments, the antimicrobial agent is phenol, m-crescol, parabens, or benzyl alcohol. In some embodiments, the multi-dose formulation includes 30 mg/ml of α-Gal A, 5% (w/v) sucrose, 5 mM citrate, 1% or less (v/v) benzyl alcohol, up to 3% (v/v) glycerol, and has a pH of 6.0. In some embodiments, α-Gal A is administered once per day, once every two days, once every three days, once every four days, once every five days, or once every six days. In a dose of from about 0.1 mg to about 20 mg of α-Gal A per kg body weight. In some embodiments, the α-Gal A formulation is a Replagal® or Fabrazyme® formulation.

According to another aspect of the invention, methods of producing therapeutically effective kidney levels of α-Gal A in an individual with Fabry disease are provided. The methods include administering subcutaneously to the individual a dose of from about 0.1 mg to about 20 mg of α-Gal A per kg, body weight, wherein the dose is administered once per day, once every two days, once every three days, once every four days, once every five days, or once every six days. In certain embodiments, the dose does not result in a toxic level of α-Gal A in the liver of the individual. In some embodiments, α-Gal A is administered in sufficient dose to result in kidney α-Gal A levels in the individual that result in an increase in the fraction of normal glomeruli and/or a decrease in the fraction of glomeruli with mesangial widening. In some embodiments, the α-Gal A is administered in sufficient dose to result in a peak concentration of α-Gal A in kidney of the subject within about 24 hours after the administration of the dose. In certain embodiments, the α-Gal A is administered in sufficient dose to result in a peak concentration of α-Gal A in kidney of the subject within about 45, 40, 35, 30, 25, or fewer hours after the administration of the dose. In some embodiments, the α-Gal A is isolated, genetically engineered α-Gal A. In some embodiments, the genetically engineered α-Gal A is produced in a human cell, a yeast cell, a bacterial cell, an insect cell, or a plant cell. In some embodiments, the α-Gal A is administered in an α-Gal A formulation. In certain embodiments, the formulation of the α-Gal A is a single dose formulation. In some embodiments, the formulation of the α-Gal A includes from about 1 mg/ml to about 60 mg/ml α Gal A, from about 2% to about 10% (w/v) carbohydrate, from about 5 mM to about 10 mM citrate, up to 3% (v/v) excipient, and from about 0.05% to about 0.5% (v/v) surfactant. In certain embodiments, the pH of the formulation is about 6.0. In some embodiments, the carbohydrate is sucrose. In some embodiments, the excipient is glycerol. In some embodiments, the surfactant is poloxamer 188. In certain embodiments, the single dose formulation includes 30 mg/ml of α-Gal A, 5% (w/v) sucrose, 5 mM citrate, between about 1% and 2.5% (v/v) glycerol, and 0.05% (v/v) poloxamer 188, and wherein the pH of the formulation is 6.0. In some embodiments, the formulation of the α-Gal A is a multi-dose formulation. In some embodiments, the formulation of the α-Gal A includes from about 1 mg/ml to about 60 mg/ml α Gal A, from about 2% to about 10% (w/v) carbohydrate, from about 5 mM to about 10 mM citrate, about 1% or less of an antimicrobial agent, and up to 3% (v/v) excipient. In some embodiments, the pH of the formulation is about 6.0. In certain embodiments, the carbohydrate is sucrose. In some embodiments, the excipient is glycerol. In some embodiments, the antimicrobial agent is phenol, m-crescol, parabens, or benzyl alcohol. In certain embodiments, the multi-dose formulation includes 30 mg/ml of α-Gal A, 5% (w/v/) sucrose, 5 mM citrate, 1% or less (v/v) benzyl alcohol, up to 3% (v/v) glycerol, and has a pH of 6.0. In some embodiments, the α-Gal A formulation is a Replagal® or Fabrazyme® formulation.

According to yet another aspect of the invention, methods of delivering to a subject a dose of α-Gal A that reaches a peak concentration of α-Gal A in kidney of the subject within about 24 or fewer hours after the administration of the dose are provided.

According to another aspect of the invention, methods of delivering to a subject a dose of α-Gal A that reaches a peak concentration of α-Gal A in kidney of the subject within about 45, 40, 35, 30, 25, or fewer hours after the administration of the dose are provided. In some embodiments of any aforementioned delivery aspect of the invention, α-Gal A is administered once per day, once every two days, once every three days, once every four days, once every five days, or once every six days. In some embodiments of any aforementioned delivery aspect of the invention, the dose does not result in a toxic level of α-Gal A in the liver of the individual. In certain embodiments of any aforementioned delivery aspect of the invention, α-Gal A is administered in sufficient dose to result in kidney α-Gal A levels in the individual that result in an increase in the fraction of normal glomeruli and/or a decrease in the fraction of glomeruli with mesangial widening. In some embodiments of any aforementioned delivery aspect of the invention, α-Gal A is isolated or genetically engineered α-Gal A. In some embodiments, the genetically engineered α-Gal A is produced in a human cell, a yeast cell, a bacterial cell, an insect cell, or a plant cell. In certain embodiments of any aforementioned delivery aspect of the invention, α-Gal A is administered at least once a day, every two days, every three days, every four days, every five days, or every six days in a dose of from about 0.1 mg/kg body weight to about 20 mg/kg body weight. In some embodiments of any aforementioned delivery aspect of the invention, the α-Gal A is administered in a formulation. In some embodiments, the formulation of the α-Gal A is a single dose formulation. In some embodiments of any aforementioned delivery aspect of the invention, the formulation of the α-Gal A includes from about 1 mg/ml to about 60 mg/ml α Gal A, from about 2% to about 10% (w/v) carbohydrate, from about 5 mM to about 10 mM citrate, up to 3% (v/v) excipient, and from about 0.05% to about 0.5% (v/v) surfactant. In certain embodiments, the carbohydrate is sucrose. In some embodiments, the excipient is glycerol. In some embodiments the surfactant is poloxamer 188. In certain embodiments, the single dose formulation includes about 30 mg/ml of α-Gal A, 5% (w/v) sucrose, 5 mM citrate, between about 1% and 2.5% (v/v) Glycerol, and 0.05% (v/v) poloxamer 188, and wherein the pH of the formulation is 6.0. In some embodiments, the formulation of the α-Gal A is a multi-dose formulation. In some embodiments, the formulation of the α-Gal A includes from about 1 mg/ml to about 60 mg/ml α Gal A, from about 2% to about 10% (w/v) carbohydrate, from about 5 mM to about 10 mM citrate, about 1% or less of an antimicrobial agent, and up to 3% (v/v) excipient. In some embodiments, the carbohydrate is sucrose. In certain embodiments, the excipient is glycerol. In some embodiments, the antimicrobial agent is phenol, m-crescol, parabens, or benzyl alcohol. In some embodiments, the multi-dose formulation includes 30 mg/ml of α-Gal A, 5% (w/v) sucrose, 5 mM citrate, 1% or less (v/v) benzyl alcohol, up to 3% (v/v) glycerol, and has a pH of 6.0. In certain embodiments, the α-Gal A formulation is a Replagal® or Fabrazyme® formulation.

According to yet another aspect of the invention, compositions that include from about 1 mg/ml to about 60 mg/ml α-Gal A, from about 2% to about 10% (w/v) carbohydrate, from about 5 mM to about 10 mM citrate, up to 3% (v/v) excipient, from about 0.05% to about 0.5% (v/v) surfactant, and having a pH of 6.0, are provided. In some embodiments, the carbohydrate is sucrose. In some embodiments, the excipient is glycerol. In certain embodiments, the surfactant is poloxamer 188.

According to another aspect of the invention, a composition that includes 30 mg/ml of α-Gal A, 5% (w/v) sucrose, 5 mM citrate, between about 1% and 2.5% (v/v) glycerol, and 0.05% (v/v) poloxamer 188, and having a pH of 6.0, is provided.

According to yet another aspect of the invention, compositions that include from about 1 mg/ml to about 60 mg/ml α-Gal A, from about 2% to about 10% (w/v) carbohydrate, from about 5 mM to about 10 mM citrate, about 1% or less of an antimicrobial agent, up to 3% (v/v) excipient, and having a pH of 6.0, are provided. In some embodiments, the carbohydrate is sucrose. In some embodiments, the excipient is glycerol. In certain embodiments, the antimicrobial agent is phenol, m-crescol, parabens, or benzyl alcohol.

According to another aspect of the invention, compositions that include 30 mg/ml of α-Gal A, 5% (w/v) sucrose, 5 mM citrate, 1% or less (v/v) benzyl alcohol, up to 3% (v/v) glycerol, and having a pH of 6.0, are provided.

### BRIEF DESCRIPTION OF THE DRAWINGS

FIG. 1 shows a graph illustrating serum pharmacokinetics of [125I]-Replagal® in rats after SC and IV administration. Graph provides a summary of all treatment groups. 5.0 mg/kg=Group A and B; 1.0 mg/kg=Group C and D; and 0.1 mg/kg=Group E and F.

FIG. 2 shows a graph illustrating tissue radioactivity after 5.0 mg/kg SC vs IV [125I]-Replagal® in rats. Data illustrated is mean+SEM for blank-corrected CPM with n=5 rats per group. Radioactivity was measured via gamma counting of tissue sample. Tissues were not homogenized prior to analysis.

FIG. 3 is a graph illustrating tissue radioactivity after 1.0 mg/kg SC vs IV [125I]-Replagal® in rats. Data illustrated is mean+SEM for blank-corrected CPM with n=5 rats per group. Radioactivity was measured via gamma counting of tissue sample. Tissues were not homogenized prior to analysis.

FIG. 4 is a graph illustrating tissue radioactivity after 0.1 mg/kg SC vs IV [125I]-Replagal® in rats. Data illustrated is mean+SEM for blank-corrected CPM with n=5 rats per group. Radioactivity was measured via gamma counting of tissue sample. Tissues were not homogenized prior to analysis.

FIG. 5 is a graph illustrating dose-matched tissue radioactivity after SC [125I]-Replagal® expressed as percent IV values. Dose-matched “percent IV” values were calculated using the following relationship: % IV=[(mean SC CPM/mg)/(mean IV CPM/mg)*100]. Radioactivity was measured via gamma counting of tissue sample. Tissues were not homogenized prior to analysis. Calculations were performed in MS Excel spreadsheets.

FIG. 6 is a graph illustrating a summary of tissue radioactivity after subcutaneous (SC) [125I]-Replagal® expressed as percent intravenous (IV) values for all dose levels. Data is a summary of all SC dose groups (B, D, F) versus all IV dose groups (A, C, E) in terms of tissue radioactivity illustrated as mean+SEM percent IV values. Calculations were performed in MS Excel spreadsheets using the relationship: [(mean SC CPM/mg)/(mean IV CPM/mg)*100].

FIG. 7 is a WinNonLin Chart—Log-Linear Serum Radioactivity vs. Time. Rsq=0.8056 Rsq_adjusted=0.7409 HL_Lambda_z=58.2377 (hr) (5 points used in calculation) Uniform Weighting.

FIGS. 8A-8B show graphs illustrating serum pharmacokinetics of 125I-Replagal® in rats after SC administration. FIG. 8A shows Blank Corrected CPM per ml vs. time and FIG. 8B shows Percent Dose vs. time.

FIGS. 9A-9I show graphs illustration tissue radioactivity after 1.0 mg/kg SC 125I-Replagal® expressed as percent dose. FIGS. 9A, 9B, 9C, 9D, 9E, 9F, 9G, 9H, and 9I are results for liver, kidneys (pooled), heart, thyroid, injection site skin (1 cm2), distal (thigh) skin (1 cm2), spleen, testes (pooled) and lungs, respectively.

FIGS. 10A-10B show graphs of serum pharmacokinetics of 125I-Replagal® in rats after IV administration. The data depicted is mean+SEM (FIG. 10A) and mean data (FIG. 10B) for 125I-Replagal® in serum following a single intravenous injection of 1 mg/kg. The best-fit lambda z line from WinNonLin noncompartmental modeling is illustrated in FIG. 10B. Nine data points were employed to provide an acceptable correlation value (R2=0.8945). Rsq=0.8945 Rsq_adjusted=0.8794 HL_Lambda_z=19.5416 (hr). 9 points used in calculation. Uniform Weighting.

FIGS. 11A-11I show graphs providing a summary of tissue radioactivity data after 1.0 mg/kg IV 125I-Replagal®. FIGS. 11A, 11B, 11C, 11D, 11E, 11F, 11G, 11H, and 11I are results for radioactivity in kidneys, heart, spleen, liver, lungs, testes, thyroid, injection site (scapular skin, and skin, respectively.

FIGS. 12A-12C show graphs providing a summary of TCA precipitable radioactivity in rat tissues 24 h after SC (FIG. 12A) or IV (FIG. 12B) injection of 1 mg/kg 125I-Replagal®. FIG. 12C provides a summary of mean pellet recovery (%) at 24 h. Data represents mean percent recovery for n=3 rats per route.

FIGS. 13A-13C show graphs providing a summary of TCA precipitable radioactivity in rat tissues 48 h after SC (FIG. 13A) or IV (FIG. 13B) injection of 1 mg/kg 125I-Replagal®. FIG. 13C provides a summary of mean pellet recovery (%) at 48 h. Data represents mean percent recovery for n=3 rats per route.

FIG. 14 is a graph of serum radioactivity in wild-type male Fabry mice after a single SC or IV injection of 1 mg/kg 125I-Replagal®. Data is mean+SEM for n=per time point per route.

FIGS. 15A-15F show graphs that provide a summary of tissue radioactivity (total organ CPM) over 24 h after a single SC or IV injection of 1 mg/kg 125I-Replagal® in wild-type male Fabry mice. Data is mean+SEM for n=3 mice per time point per route. FIGS. 15A, 15B, 15C, 15D, 15E, and 15F are results for radioactivity in kidney, liver, heart, spleen, thyroid, and injection site (SC group only), respectively.

FIGS. 16A-16F show graphs that provide a summary of tissue radioactivity (percent dose) over 24 h after a single SC (dark columns) or IV injection (light columns) of 1 mg/kg 125I-Replagal® in wild-type male Fabry mice. Data is mean+SEM for n=3 mice per time point per route. FIGS. 16A, 16B, 15C, 16D, 16E, and 16F are results for radioactivity in kidney, liver, heart, spleen, thyroid, and injection site (SC group only), respectively.

FIGS. 17A-17B show graphs that provide a summary of TCA-precipitable radioactivity in representative liver and kidney samples from mice injected with SC or IV 1 mg/kg 125I-Replagal®. FIG. 17A shows percent recovery for SC and IV for kidney and liver. FIG. 17B shows a summary of mean pellet and supernatant recovery in selected tissues. Data is mean of n=2 tissues per group.

FIG. 18 is a graph showing serum radioactivity in Groups A and B after 2×1 mg/kg 125I-Replagal® in rats. Data represents n>3 rats per point. Vertical lines indicate dosing times after initial dose at t=0 h.

FIG. 19 is a graph showing serum radioactivity in Groups C and D after 4×1 mg/kg 125I-Replagal® in rats. Data represents n>3 rats per point. Vertical lines indicate dosing times after initial dose at t=0 h.

FIG. 20 is a graph showing serum radioactivity in Groups A and B after 2×0.5 mg/kg 125I-Replagal® in rats. Data represents n>3 rats per point. Vertical lines indicate dosing times after initial dose at t=0 h.

FIG. 21 is a graph showing serum radioactivity in Groups A and B after 4×0.25 mg/kg 125I-Replagal® in rats. Data represents n>3 rats per point. Vertical lines indicate dosing times after initial dose at t=0 h.

FIGS. 22A-22F show graphs illustrating tissue radioactivity after 2×1 mg/kg 125I-Replagal® in rats expressed as percent dose. X-axis is time after study was initiated; since these animals received the second and final dose at 96 hrs, the time points below represent 24 hr, 48 hr, and 72 hr after the last treatment. Data is mean±SEM for n=3 rats per time point. FIGS. 22A, 22B, 22C, 22D, 22E, and 22F are results in kidney, liver, heart, spleen, thyroid, and injection site (skin), respectively.

FIGS. 23A-23F show graphs illustrating tissue radioactivity after 4×1 mg/kg 125I-Replagal® in rats expressed as percent dose. X-axis is time after study was initiated; these animals received injections at 0, 48, 96, and 144 hrs. These data represent tissue radioactivity 24 hr after the second, third, and final treatments. Data is mean±SEM for n=3 rats per time point. FIGS. 23A, 23B, 23C, 23D, 23E, and 23F are results for kidney, liver, heart, spleen, thyroid, and injection site (skin), respectively.

FIGS. 24A-24F show graphs illustrating tissue radioactivity after 2×0.5 mg/kg 125I-Replagal® in rats expressed as percent dose. X-axis is time after study was initiated; since these animals received the second and final dose at 96 hrs, the time points below represent 24 hr, 48 hr, and 72 hr after the last treatment. Data is mean±SEM for n=3 rats per time point. FIGS. 24A, 24B, 24C, 24D, 24E, and 24F are results for kidney, liver, heart, spleen, thyroid, and injection site (skin), respectively.

FIGS. 25A-25F show graphs illustrating tissue radioactivity after 4×0.25 mg/kg 125I-Replagal® in rats expressed as percent dose. X-axis is time after study was initiated; animals received the final injection at 96 hrs. Data is mean±SEM for n=3 rats per time point. FIGS. 25A, 25B, 25C, 25D, 25E, and 25F are results for kidney, liver, heart, spleen, thyroid, and injection site (skin), respectively.

FIGS. 26A-26B show a graph (FIG. 26A) and table (FIG. 26B) illustrating serum radioactivity over one week after a single injection of 1 mg/kg 125I-Replagal® in JVC rats. Data represents mean+SEM for n=>3 rats per route group. AUCobs=observed area under the CMP per mL vs. time curve. F %=fraction available, i.e., bioavailability.

FIG. 27 shows a summary of WinNonLin NCA results for serum radioactivity after a single 1 mg/kg injection of 125I-Replagal® in rats. Lambda z=elimination constant; Lambda z t_{1/2}=elimination (terminal phase) serum half-life; Tₘₐₓ=time maximal serum CPM/mL is achieved following injection; Cₘₐₓ=maximal serum CPM/mL achieved following injection; AUC_{obs}=observed area under the CPM/mL vs. time curve, without extrapolation; AUC_{inf}=area under the CPM/mL vs. time curve, with extrapolation to infinity; Vz_{obs}=volume of distribution in the terminal phase (z); Cl z_{obs}=serum clearance in the terminal phase (z); AUMC_{obs}=observed area under the first moment curve without extrapolation; AUMC_{inf}=area under the first moment curve with extrapolation to infinity; MRT_{obs}=observed mean residence time without extrapolation; MRT_{inf}=observed mean residence time with extrapolation to infinity; .Fobs=observed fraction available (bioavailability), based on the observed AUC; and F_{inf}=fraction available (bioavailability), based on AUC extrapolated to infinity.

FIG. 28A is a graph showing that after a single 1 mg/kg 125I-Replagal® injection, radioactivity levels peaked in kidney at 24 hr after subcutaneous (SC) injection compared to 48 hr following intravenous (IV) injection. These data are expressed as the mean percent dose (±SEM) for n=3 rats per time point. FIG. 28A shows the percent dose vs. time after injection (hr). FIG. 28B shows Cmax and time to Cmax for SC and IV route.

### DETAILED DESCRIPTION

It has been discovered that human α-Gal A can be made having modifications (e.g., in carbohydrate structure, e.g., glycan, phosphate or sialylation modifications) that result in a human α-Gal A preparation having pharmacokinetic properties that are desirable for enzyme replacement therapy for α-Gal A deficiency. For example, a preparation of human α-Gal A produced from human cells genetically engineered to produce human α-Gal A has an exponent “b” for the allometric scaling equation for clearance from the circulation in humans, Y=a(BW)^{b}, of at least 0.85 (in some embodiments, up to 0.92), where Y is clearance rate of α-Gal A (ml/min), “a” is a non-specific constant, and BW is body weight. Such an α-Gal A preparation, as described herein, can be predominantly taken up by M6P receptors and has a serum clearance less rapid than that of human ab-Gal A produced in non-human cells, e.g., CHO cells. Accordingly, pharmaceutical compositions and kits for treatment of α-Gal A deficiency described herein include such α-Gal A preparations that are administered in a unit dose substantially smaller than what is currently used in the art. For example, in some embodiments, the α-Gal A preparations described herein are administered in a unit dose of between 0.05 mg and 2.0 mg per kilogram of body weight (mg/kg), in some embodiments, between 0.05 and 5 mg/kg, in certain embodiments, between 0.05 and 0.3 mg/kg (e.g., about 0.1, 0.2, 0.25, 0.3, 0.4 or 0.5 mg/kg). The unit dose can be, e.g., between 0.1×10⁶ U/kg and 10×10⁶ U/kg. In some embodiments, the unit dose of the α-Gal A preparation is between 0.1×10⁶ U/kg and 5×10⁶ U/kg, and in certain embodiments, the unit does is between about 0.1×10⁶ U/kg and 3×10⁶ U/kg. In some embodiments of the invention, the α-Gal A preparations described herein are administered at a frequency of about every day, every two days, every three days, every four days, every five days, or every six days.

It is believed that the desirable pharmacokinetics result at least in part from the glycosylation patterns of the α-Gal A preparation. The glycosylation patterns required for the desirable pharmacokinetics of human α-Gal A (e.g., at least 50% complex glycans per α-Gal A monomer, on average; a ratio of sialic acid to mannose-6-phosphate on a mole per mole basis) greater than 1.5 to 1, in some embodiments, greater than 2 to 1, in certain embodiments, greater than 3 to 1, and in some embodiments, greater than 3.5 to 1 or higher) can be achieved through a number of methods known in the art. Certain representative embodiments are summarized and described in greater detail below.

The α-Gal A preparations described herein can be produced in any cell (an α-Gal A production cell) for the treatment of Fabry disease. In some embodiments, the compositions and methods described herein use human α-Gal A produced using standard genetic engineering techniques (based on introduction of the cloned α-Gal A gene or cDNA into a host cell), or gene activation, described in more detail below. The human α-Gal A can be produced in human cells, which provide the carbohydrate modifications that are important for the enzyme's pharmacokinetic activity.

However, human α-Gal A can also be produced in non-human cells, e.g., CHO cells. If the α-Gal A is produced in non-human cells, one or more of: the α-Gal A expression construct, the non-human cells, or the α-Gal A isolated from the non-human cells can be modified, e.g., as described herein below, to provide α-Gal A preparations having a glycosylation profile that results in desirable pharmacokinetic properties.

The term α-Galactosidase A (α-Gal A) is also known in the art as: a-D-galactoside galactohydrolase, algalsidase alpha, a-D-galactosidase, α-Gal, Gal A, α-galactosidase A, α-galactoside galactohydrolase, melibiase, TmGa1A, TnGa1A, and algalsidase beta. Commercially available forms of α-Gal A include Replagal® and Fabrazyme®. A function of α-gal A is catalysis of the hydrolysis of globotriaosy-lceramide (G13) and other α-galactyl-terminated neutral glycosphingolipids, such as galabiosylceramide and blood group B substances to ceramide dihexoside and galactose.

### Cells Suitable for Production of Human α-Gal A

Purified human α-Gal A can be obtained from cultured cells, in some embodiments, genetically modified cells, e.g., genetically modified human cells or other mammalian cells, e.g., CHO cells. Insect cells and plant cells, e.g., carrot plant root cells, can also be used. When cells are to be genetically modified for the purposes of treatment of Fabry disease, the cells may be modified by conventional genetic engineering methods or by gene activation.

According to conventional methods, a DNA molecule that contains an α-Gal A cDNA or genomic DNA sequence may be contained within an expression construct and transfected into primary, secondary, or immortalized cells by standard methods including, but not limited to, liposome-, polybrene-, or DEAE dextran-mediated transfection, electroporation, calcium phosphate precipitation, microinjection, or velocity driven microprojectiles (see, e.g., U.S. Pat. No. 6,048,729, incorporated herein by reference).

Alternatively, one can use a system that delivers the genetic information by viral vector. Viruses known to be useful for gene transfer include adenoviruses, adeno associated virus, herpes virus, mumps virus, poliovirus, retroviruses, Sindbis virus, and vaccinia virus such as canary pox virus. Alternatively, cells may be genetically modified using a gene activation (“GA”) approach, for example, as described in U.S. Pat. No. 5,641,670; U.S. Pat. No. 5,733,761; U.S. Pat. No. 5,968,502; U.S. Pat. No. 6,200,778; U.S. Pat. No. 6,214,622; U.S. Pat. No. 6,063,630; U.S. Pat. No. 6,187,305; U.S. Pat. No. 6,270,989; and U.S. Pat. No. 6,242,218, each incorporated herein by reference. α-Gal A made by gene activation is referred to herein as GA-GAL (Selden et al., U.S. Pat. Nos. 6,083,725 and 6,458,574 B1).

The term “genetically modified,” as used herein in reference to cells, is meant to encompass cells that express a particular gene product following introduction of a DNA molecule encoding the gene product and/or including regulatory elements that control expression of a coding sequence for the gene product. The DNA molecule may be introduced by gene targeting or homologous recombination, i.e., introduction of the DNA molecule at a particular genomic site. Homologous recombination may be used to replace the defective gene itself (the defective α-Gal A gene or a portion of it could be replaced in a Fabry disease patient's own cells with the whole gene or a portion thereof).

In some aspects of the invention, cells that produce α-Gal A are cells that are genetically engineered cells. As used herein, the term “genetically engineered” means cells that have been genetically altered by the introduction of heterologous DNA (RNA) encoding an α-Gal A polypeptide or fragment or variant thereof into the cells. The introduced heterologous DNA (RNA) is placed under operable control of transcriptional elements to permit the expression of the heterologous DNA in the host cell. In certain embodiments of the invention, genetically engineered α-Gal A is α-Gal A that is recombinantly produced.

Cells of the invention are maintained under conditions, as are known in the art that result in expression of the α-Gal A polypeptide or functional fragments thereof. Polypeptides expressed using any suitable method, including, but not limited to methods described herein, may be purified from cell lysates or cell supernatants. Polypeptides made according to methods set forth herein or by alternative methods can be prepared as a pharmaceutically useful formulation and delivered to a human or non-human animal by conventional pharmaceutical routes as is known in the art (e.g., subcutaneous). As described elsewhere herein, recombinant cells can be immortalized, primary, or secondary cells, preferably human. The use of cells from other species may be desirable in cases where the non-human cells are advantageous for polypeptide production purposes where the non-human α-Gal A produced is useful therapeutically. Cell-free transcription systems also may be used in lieu of cells.

As used herein, the term “primary cell” includes cells present in a suspension of cells isolated from a vertebrate, or other, tissue source (prior to their being plated, i.e., attached to a tissue culture substrate such as a dish or flask), cells present in an explant derived from tissue, both of the previous types of cells plated for the first time, and cell suspensions derived from these plated cells.

“Secondary cells” refers to cells at all subsequent steps in culturing. That is, the first time a plated primary cell is removed from the culture substrate and replated (passaged), it is referred to as a secondary cell, as are all cells in subsequent passages.

A “cell strain” consists of secondary cells which have been passaged one or more times; exhibit a finite number of mean population doublings in culture; exhibit the properties of contact-inhibited, anchorage dependent growth (except for cells propagated in suspension culture); and are not immortalized.

By “immortalized cell” or “continuous cell line” is meant a cell from an established cell line that exhibits an apparently unlimited lifespan in culture.

Examples of primary or secondary cells include fibroblasts, epithelial cells including mammary and intestinal epithelial cells, endothelial cells, formed elements of the blood including lymphocytes and bone marrow cells, glial cells, hepatocytes, keratinocytes, muscle cells, neural cells, or the precursors of these cell types. Examples of immortalized human cell lines useful in the present methods include, but are not limited to, Bowes Melanoma cells (ATCC Accession No. CRL 9607), Daudi cells (ATCC Accession No. CCL 213), HeLa cells and derivatives of HeLa cells (ATCC Accession Nos. CCL 2, CCL 2. 1, and CCL 2.2), HL-60 cells (ATCC Accession No. CCL 240), HT-1080 cells (ATCC Accession No. CCL 121), Jurkat cells (ATCC Accession No. TIB 152), KB carcinoma cells (ATCC Accession No. CCL 17), K-562 leukemia cells (ATCC Accession No. CCL 243), MCF-7 breast cancer cells (ATCC Accession No. BTH 22), MOLT-4 cells (ATCC Accession No. 1582), Namalwa cells (ATCC Accession No. CRL 1432), Raji cells (ATCC Accession No. CCL 86), RPMI 8226 cells (ATCC Accession No. CCL 155), U-937 cells (ATCC Accession No. CRL 15 93), WI-3 8VAI 3 sub line 2R4 cells (ATCC Accession No. CLL 75. 1), CCRF-CEM cells (ATCC Accession No. CCL 119), and 2780AD ovarian carcinoma cells (Van der Blick et al., Cancer Res. 48: 5927-5932, 1988), as well as heterohybridoma cells produced by fusion of human cells and cells of another species.

Following the genetic modification of human cells to produce a cell which secretes α-Gal A, a clonal cell strain consisting essentially of a plurality of genetically identical cultured primary human cells or, where the cells are immortalized, a clonal cell line consisting essentially of a plurality of genetically identical immortalized human cells, may be generated. In one embodiment, the cells of the clonal cell strain or clonal cell line are fibroblasts. In some embodiments the cells are secondary human fibroblasts, e.g., BRS-L11 cells.

Additional guidance on the production of cells genetically engineered to produce human α-Gal A.

After genetic modification, the cells are cultured under conditions permitting production and secretion of α-Gal A. The protein is isolated from the cultured cells by collecting the medium in which the cells are grown, and/or lysing the cells to release their contents, and then applying protein purification techniques.

Increasing Circulatory Half Life, Cellular Uptake and/or Targeting of α-Gal A to Appropriate Tissues

The data described herein shows that human α-Gal A can be made having modifications (e.g., carbohydrate, phosphate or sialylation modifications) that result in pharmacokinetic properties of the enzyme that are desirable for use in enzyme replacement therapy for α-Gal A deficiency. One method of making such human α-Gal A preparations is to produce human α-Gal A from human cells.

There are differences in the glycosylation characteristics of human and nonhuman cells (e.g., CHO cells) such that, the production of α-Gal A (or indeed, of any glycoprotein) from human cells necessarily results in a structurally different protein than that produced in CHO cells. Although not bound by theory, these differences are thought to be important for the desirable pharmacokinetics of human α-Gal A preparations in the compositions and methods described herein. However, α-Gal A preparations described herein can also be produced from non-human cells, wherein either the cells, the α-Gal A coding sequence and/or the purified α-Gal A are modified. For example, non-human cells whose glycosylation machinery differs from human (e.g., CHO cells) can be genetically modified to express an enzyme of carbohydrate metabolism, e.g., α-2,6-sialyltransferase, that is present in human but not in CHO cells.

In another example, the cells can be genetically engineered to express an α-Gal A protein that has one or more modified glycosylation sites, e.g., a human or non-human cell can be genetically engineered to express an α-Gal A coding sequence in which one or more additional N-linked glycosylation sites have been added or deleted. The additional glycosylation sites can be glycosylated by the cellular machinery in the cell, e.g., the CHO cell, in which the modified α-Gal A coding sequence is expressed, thus providing “an α-Gal A preparation” that has an increased circulatory half-life, cellular uptake, and/or improved targeting to heart, kidney or other appropriate tissues compared to the unmodified α-Gal A, e.g., when expressed in non-human cells.

α-Gal A can also be modified (e.g., after isolation from a genetically engineered non-human cell) to resemble human α-Gal A produced in human cells. For example, a human α-Gal A preparation isolated from a non-human cell can be modified, e.g., phosphorylated or cleaved (e.g., with neuraminidase or phosphatase) before administration to a subject.

The circulating half-life, cellular uptake and/or tissue targeting can also be modified, inter alia, by (i) modulating the phosphorylation of α-Gal A; (ii) modulating the sialic acid content of α-Gal A; and/or (iii) sequential removal of the sialic acid and terminal galactose residues, or removal of terminal galactose residues, on the oligosaccharide chains on α-Gal A. Altered sialylation of α-Gal A preparations can enhance the circulatory half-life, cellular uptake and/or tissue targeting of exogenous α-Gal A. A change in the ratio of moles of mannose-6-phosphate per mole of sialic acid per molecule of α-Gal A can also result in improved cellular uptake, relative to that of hepatocytes, in non-hepatocytes such as liver endothelial cells, liver sinusoidal-cells, capillary/vascular endothelial cells, renal glomerular epithelial cells (podocytes) and glomerular mesangial cells, renal endothelial cells, pulmonary cells, renal cells, neural cells, and/or cardiac myocytes. For example, in some embodiments, a ratio of sialic acid to mannose-6-phosphate in the α-Gal A preparation (on a mole per mole basis) is greater than 1.5 to 1, in some embodiments, greater than 2 to 1, in certain embodiments, greater than 3 to 1, and in some embodiments, the ratio is greater than 3.5 to 1 or higher.

### Glycan Remodeling

Glycoprotein modification (e.g., when α-Gal A is produced in non-human cells) can increase uptake of the enzyme in specific tissues other than liver and macrophages, e.g., increase uptake in capillary/vascular endothelial cells, renal glomerular epithelial cells (podocytes) and glomerular mesangial cells, renal endothelial cells, pulmonary cells, renal cells, neural cells, and/or cardiac myocytes. Using glycoprotein modification methods, human glycosylated α-Gal A preparations can be obtained, wherein between 35% and 85% of the oligosaccharides, in some embodiments, at least 50%, are charged.

Protein N-glycosylation functions by modifying appropriate asparagine residues of proteins with oligosaccharide structures, thus influencing their properties and bioactivities (Kukuruzinska & Lennon, Crit. Rev. Oral. Biol. Med. 9: 415-48 (1998)). An α-Gal A preparation described herein can have a high percentage of the oligosaccharides being negatively charged, primarily by the addition of one to four sialic acid residues on complex glycans, or of one to two phosphate moieties on high-mannose glycans, or of a single phosphate and a single sialic acid on hybrid glycans. Smaller amounts of sulfated complex glycans may also be present. A high proportion of charged structures serves two main functions. First, capping of penultimate galactose residues by 2,3- or 2,6-linked sialic acid prevents premature removal from the circulation by the asialoglycoprotein receptor present on hepatocytes. This receptor recognizes glycoproteins with terminal galactose residues.

Modifying the glycosylation pattern of α-Gal A produced in non-human cells to, e.g., resemble the pattern produced in human cells, gives important target organs such as heart and kidney the opportunity to endocytose greater amounts of enzyme from the plasma following enzyme administration. Second, the presence of Man-6-phosphate on high-mannose or hybrid glycans provides an opportunity for receptor-mediated uptake by the cation-independent Man-6-phosphate receptor (CI-MPR). This receptor-mediated uptake occurs on the surface of many cells, including vascular endothelial cells, which are a major storage site of Gb3 in Fabry patients. Enzyme molecules with two Man-6-phosphate residues have a much greater affinity for the CI-MPR than those with a single Man-6-phosphate.

The complexity of N-glycosylation is augmented by the fact that different asparagine residues within the same polypeptide may be modified with different oligosaccharide structures, and various proteins are distinguished from one another by the characteristics of their carbohydrate moieties.

Several approaches are provided herein for carbohydrate remodeling on a protein containing N-linked glycan chains. First, one can genetically engineer a cell, e.g., a non-human cell, to produce a human α-Gal A having a non-naturally occurring glycosylation site, e.g., one can engineer a human α-Gal A coding sequence to produce an α-Gal A protein having one or more additional glycosylation sites. The additional glycosylation sites can be glycosylated (e.g., with complex glycans) by the cellular machinery in the cell, e.g., the CHO cell, in which the modified α-Gal A coding sequence is expressed, thus providing an α-Gal A preparation that has improved circulatory half-life, cellular uptake and/or tissue targeting compared to the unmodified α-Gal A, e.g., when expressed in non-human cells.

Second, the proportion of charged α-Gal A can be increased by selective isolation of glycoforms during the purification process. The present invention provides for increasing the proportion of highly charged and higher molecular weight α-Gal A glycoforms by fractionation of α-Gal A species on chromatography column resins during and/or after the purification process. The more highly charged glycoform species of α-Gal A contain more sialic acid and/or more phosphate, and the higher molecular weight glycoforms would also contain the fully glycosylated, most highly branched and highly charged species. Selection of the charged species, or removal of the non-glycosylated, poorly glycosylated or poorly sialylated and/or phosphorylated α-Gal A species would result in a population of α-Gal A glycoforms with more sialic acid and/or a more desirable sialic acid to phosphate ratio in the preparation, therefore providing an α-Gal A preparation with better half-life, cellular uptake and/or tissue targeting, thereby having better therapeutic efficiency.

This fractionation process can occur on, but is not limited to, suitable chromatographic column resins utilized to purify or isolate α-Gal A. For example, fractionation can occur on, but is not limited to, cation exchange resins (such as SP-SepharoseG), anion exchange resins (Q-SepharoseG), affinity resins (Heparin Sepharose-b, lectin columns) size exclusion columns (Superdex 200) and hydrophobic interaction columns (Butyl Sepharose); and other chromatographic column resins known in the art.

Because α-Gal A is produced in cells as a heterogeneous mixture of glycoforms that differ in molecular weight and charge, α-Gal A tends to elute in relatively broad peaks from the chromatography resins. Within these elutions, the glycoforms are distributed in a particular manner depending on the nature of the resin being utilized. For example, on size exclusion chromatography, the largest glycoforms will tend to elute earlier on the elution profile than the smaller glycoforms. On ion exchange chromatography, the most negatively charged glycoforms will tend to bind to a positively charged resin (such as Q-SepharoseG) with higher affinity than the less negatively charged glycoforms, and will therefore tend to elute later in the elution profile. In contrast, these highly negatively charged glycoforms may bind less tightly to a negatively charged resin, such as SP Sepharose8, than less negatively charges species, or may not even bind at all.

Fractionation and selection of highly charged and/or higher molecular weight glycoforms of α-Gal A can be performed on any α-Gal A preparation, such as that derived from genetically modified cells such as cells, e.g., human or non-human cells, modified by conventional genetic engineering methods or by gene activation (GA). It can be performed on cell lines grown in optimized systems to provide altered sialylation and phosphorylation as described herein, e.g., to provide a preparation with a ratio of sialic acid to mannose-6-phosphate (on a mole per mole basis) is greater than 1.5 to 1, in some embodiments greater than 2 to 1, in certain embodiments, greater than 3 to 1 and in some embodiments, greater than 3.5 to 1 or higher.

A third approach for carbohydrate remodeling can involve modifying certain glycoforms on the purified α-Gal A by attachment of an additional terminal sugar residue using a purified glycosyl transferase and the appropriate nucleotide sugar donor. This treatment affects only those glycoforms that have an appropriate free terminal sugar residue to act as an acceptor for the glycosyl transferase being used. For example, α2,6-sialyltransferase adds sialic acid in an α-2,6-linkage onto a terminal Galβ1,4GlcNAc-R acceptor, using CMP-sialic acid as the nucleotide sugar donor. Commercially available enzymes and their species of origin include: fucose α1,3 transferases III, V and VI (humans); galactose α1,3 transferase (porcine); galactose β1,4 transferase (bovine); mannose α1,2 transferase (yeast); sialic acid α2,3 transferase (rat); and sialic acid α2,6 transferase (rat). After the reaction is completed, the glycosyl transferase can be removed from the reaction mixture by a glycosyl transferase specific affinity column consisting of the appropriate nucleotide bonded to a gel through a 6 carbon spacer by a pyrophosphate (GDP, UDP) or phosphate (CMP) linkage or by other chromatographic methods known in the art. Of the glycosyl transferases listed above, the sialyl transferases are particularly useful for modification of enzymes, such as α-Gal A, for enzyme replacement therapy in human patients. Use of either sialyl transferase with CMP-5-fluoresceinyl-neuraminic acid as the nucleotide sugar donor yields a fluorescently labeled glycoprotein whose uptake and tissue localization can be readily monitored.

A fourth approach for carbohydrate remodeling involves glyco-engineering, e.g., introduction of genes that affect glycosylation mechanisms of the cell, of the α-Gal A production cell to modify post-translational processing in the Golgi apparatus is an approach in some embodiments.

A fifth approach for carbohydrate remodeling involves treating α-Gal A with appropriate glycosidases to reduce the number of different glycoforms present. For example, sequential treatment of complex glycan chains with neuraminidase, β-galactosidase, and β-hexosaminidase cleaves the oligosaccharide to the trimannose, core.

A sixth approach for glycan remodeling involves the use of inhibitors of glycosylation, e.g., kifunensine (an inhibitor of mannosidase I), swainsonine, or the like. Such inhibitors can be added to the cultured cells expressing a human α-Gal A. The inhibitors are taken up into the cells and inhibit glycosylation enzymes, such as glycosyl transferases and glycosidases, providing α-Gal A molecules with altered sugar structures. Alternatively, a cell genetically engineered to produce human α-Gal A can be transfected with glycosylation enzymes Such as glycosyl transferases and glycosidases.

A seventh approach involves using glycosylation enzymes (e.g., glycosyl transferases or glycosidases) to remodel the carbohydrate structures in vitro, e.g., on an α-Gal A that has been isolated from a genetically engineered cell, as described herein. Other approaches for glycan remodeling are known in the art.

Altering Half Life and/or Cellular Uptake of α-Gal A by Altering Sialylation

Sialylation affects the circulatory half-life and biodistribution of proteins. Proteins with minimal or no sialic acid are readily internalized by the asialoglycoprotein receptor (Ashwell receptor) on hepatocytes by exposed galactose residues on the protein. The circulating half-life of galactose-terminated α-Gal A can be altered by sequentially (1) removing sialic acid by contacting α-Gal A with neuraminidase (sialidase), thereby leaving the terminal galactose moieties exposed, and (2) removing the terminal galactoside residues by contacting the desialylated α-Gal A with β-galactosidase. The resulting α-Gal A preparation has a reduced number of terminal sialic acid and/or terminal galactoside residues on the oligosaccharide chains compared to α-Gal A preparations not sequentially contacted with neuraminidase and β-galactosidase. Alternatively, the circulating half-life of galactose-terminated α-Gal A can be enhanced by only removing the terminal galactoside residues by contacting the desialylated α-Gal A with β-galactosidase. The resulting α-Gal A preparation has a reduced number of terminal galactoside residues on the oligosaccharide chains compared to α-Gal A preparations not contacted with β-galactosidase. In some embodiments, following sequential contact with neuraminidase and β-galactosidase, the resulting α-Gal A preparations are subsequently contacted with β-hexosaminidase, thereby cleaving the oligosaccharide to the trimannose core.

The sialic acid content of α-Gal A preparations can be increased by (i) isolation of the highly charged and/or higher molecular weight α-Gal A glycoforms during or after the purification process; (ii) adding sialic acid residues using cells genetically modified (either by conventional genetic engineering methods or gene activation) to express a sialyl transferase gene or cDNA; or (iii) fermentation or growth of cells expressing the enzyme in a low ammonium environment.

Altering Half Life and/or Cellular Uptake by Altering Phosphorylation

Altering the phosphorylation of an α-Gal A preparation described herein can alter the circulatory half life and cellular uptake of the preparation into desired tissues. In some embodiments, an α-Gal A preparation has less than 45% phosphorylated glycans. For example, the preparation has less than about 35%, 30%, 25%, or 20% phosphorylated glycans. A desirable ratio of sialic acid:mannose-6-phosphate in the α-Gal A preparation (on a mole per mole basis) is a ratio greater than 1.5 to 1, in some embodiments, greater than 2 to 1, in certain embodiments, greater than 3 to 1, and in some embodiments greater than 3.5 to 1 or higher.

The phosphorylation of α-Gal A preparations can be modified, e.g., increased or decreased, by (i) adding or removing phosphate residues using cells genetically modified (either by conventional genetic engineering methods or gene activation) to express a phosphoryl transferase or phosphatase gene or cDNA; (ii) adding phosphatases, kinases, or their inhibitors to the cultured cells; or (iii) adding phosphatases kinases, or their inhibitors to a purified α-Gal A preparation produced from a genetically engineered cell as described herein.

The concerted actions of two membrane-bound Golgi enzymes are needed to generate a Man-6-phosphate recognition marker on a lysosomal proenzyme. The first, UDP-N-acetylglucosamine: glycoprotein N-acetylglucosamine-l-phosphotransferase (G1cNAc phosphotransferase), requires a protein recognition determinant on lysosomal enzymes that consists of two lysine residues 34Å apart and in the correct spatial relationship to a high mannose chain. The second, N-acetylglucosamine-1-phosphodiester α-N-acetylglucosaminidase (phosphodiester x-GlcNAcase), hydrolyzes the x-GlcNAc-phosphate bond exposing the Man-6-phosphate recognition site. These enzymes can be induced or inhibited by methods known in the art to provide an α-Gal A preparation with desirable phosphorylation characteristics (e.g., with a desirable ration of sialylated to phosphorylated glycans).

In one embodiment, an α-Gal A preparation with altered phosphorylation is obtained by first introducing into an α-Gal A production cell a polynucleotide that encodes for phosphoryl transferase, or by introducing a regulatory sequence by homologous recombination that regulates expression of an endogenous phosphoryl transferase gene. The α-Gal A production cell is then cultured under culture conditions that result in expression of α-Gal A and phosphoryl transferase. The α-Gal A preparation with increased phosphorylation compared to the α-Gal A produced in a cell without the polynucleotide is then isolated.

In still another embodiment, a glycosylated α-Gal A preparation with altered phosphorylation is obtained by adding a phosphatase inhibitor, e.g., bromotetramisole, or a kinase inhibitor, to cultured cells.

Using the methods described herein, α-Gal A preparations are obtained wherein at doses below serum or plasma clearance saturation levels, serum clearance of the α-Gal A preparation from the circulation is in some embodiments, less than 4 mL/min/kg on the linear portion of the AUC vs. dose curve, in certain embodiments, less than about 3.5, 3, or 2.5 mL/min/kg, on the linear portion of the AUC vs. dose curve. The α-Gal A preparation has an exponent “b” for the allometric scaling equation for clearance from the circulation in mammals, Y=(BW)^{b}, of at least 0.85, where Y is clearance of α-Gal A from the circulation (ml/min), “a” is a non-specific constant and BW is body weight. The exponent “b” is in some embodiments at least 0.88, in certain embodiments, at least 0.90, and in some embodiments, at least 0.92, 0.94 or higher.

In some embodiments, an α-Gal A preparation described herein is enriched in neutral, mono-sialylated and di-sialylated glycan structures (combined) relative to more highly sialylated structures such as tri-sialylated and tetra-sialylated structures. For example, in some embodiments, an α-Gal A preparation has one or more of: (a) at least about 22% neutral glycans, e.g., at least about 25% or 30% neutral glycans; (b) at least about 15%, 20%, or 25% mono-sialylated glycans; (c) at least about 35%, in some embodiments, at least about 40%, 45%, or 50% neutral and mono-sialylated glycans combined; (d) at least about 75%, 76%, 78% or more neutral, mono- and di-sialylated glycans combined; and (e) less than about 35%, in some embodiments, less than about 25%, 20%, 18% or about 15% tri- and tetra-sialylated glycan structures combined.

In some embodiments, an α-Gal A preparation described herein has, on average, more than one complex glycan per monomer, in certain embodiments, at least 50% complex glycans per monomer, e.g., 2 complex glycans or more per monomer.

In some embodiments, an α-Gal A preparation described herein has at least 5%, and in certain embodiments, at least 7%, 10% or 15% neutral glycans.

In some embodiments, an α-Gal A preparation described herein has less than 45% phosphorylated glycans. For example, the preparation has less than about 35%, 30%, 25%, or 20% phosphorylated glycans.

In some embodiments, an α-Gal A preparation described herein has a total proportion of sialylated glycans greater than about 45%, e.g., greater than 50% or 55%.

In certain embodiments, the ratio of sialic acid to manose-6-phosphate in the α-Gal A preparation (on a mole per mole basis) is greater than 1.5 to 1, in some embodiments, greater than 2 to 1, in certain embodiments, greater than 3 to 1, and in some embodiments, the ratio is greater than 3.5 to 1 or higher.

In one embodiment, the percent ratio of sialylated glycans to phosphorylated glycans is greater than 1, in some embodiments greater than 1.5, and in certain embodiments greater than 2, e.g., greater than about 2.5 or 3.

### PEGylation

In other embodiments, the circulatory half-life of a human α-Gal A preparation is enhanced by complexing α-Gal A with polyethylene glycol (PEG). In some embodiments, the α-Gal A preparation is complexed using tresyl monomethoxy PEG (TMPEG) to form a PEGylated-α-Gal A. The PEGylated-α-Gal A is then purified to provide an isolated, PEGylated-α-Gal A preparation PEGylation of α-Gal A increases the circulating half-life, cellular uptake and/or tissue distribution of the protein.

Purification of α-Gal A from the Conditioned Medium of Stably Transfected Cells

α-Gal A may be purified to near-homogeneity from the cultured cell strains and/or conditioned medium of the cultured cell strains that have been stably transfected to produce the enzyme. α-Gal A can be isolated from α-Gal A containing media using chromatographic steps. For example, 1 or more, e.g., 2, 3, 4, 5 or more chromatographic steps can be used. The different steps of chromatography utilize various separation principles which take advantage of different physical properties of the enzyme to separate α-Gal A from contaminating material. For example, the steps can include: hydrophobic interaction chromatography on butyl Sepharose, ionic interaction on hydroxyapatite, anion exchange chromatography on Q Sepharose and size exclusion chromatography on Superdex 200, etc.. Size exclusion chromatography can serve as an effective means to exchange the purified protein into a formulation-compatible buffer.

One purification process includes the use of butyl sepharose® chromatography as a first step in purification. Other hydrophobic interaction resins, such as Source Iso (Pharmacia), Macro-Prep® Methyl Support (Bio-Rad), TSK Butyl (Tosohaas) or Phenyl Sepharose® (Pharmacia) can also be used. The column can be equilibrated in a relatively high concentration of a salt, e.g., 1 M ammonium sulfate or 2 M sodium chloride, e.g., in a buffer of pH 5.6. The sample to be purified can be prepared by adjusting the pH and salt concentration to those of the equilibration buffer. The sample is applied to the column and the column is washed with equilibration buffer to remove unbound material. The α-Gal A is eluted from the column with a lower ionic strength buffer, water, or organic solvent in water, e.g., 20% ethanol or 50% propylene glycol. Alternatively, the α-Gal A can be made to flow through the column by using a lower concentration of salt in the equilibration buffer and in the sample or by using a different pH. Other proteins may bind to the column, resulting in purification of the α-Gal A-containing sample which did not bind the column.

An alternative step of purification can use a cation exchange resin, e.g., SP Sepharose® 6 Fast Flow (Pharmacia), Source 30S (Pharmacia), CM Sepharose® Fast Flow (Pharmacia), Macro-Prep® CM Support (Bio-Rad) or Macro-Prep® High S Support (Bio-Rad), to purify α-Gal A. The “first chromatography step” is the first application of a sample to a chromatography column (all steps associated with the preparation of the sample are excluded). The α-Gal A can bind to the column at pH 4.4. A buffer, such as 10 mM sodium acetate, pH 4.4, 10 mM sodium citrate, pH 4.4, or other buffer with adequate buffering capacity at approximately pH 4.4, can be used to equilibrate the column. The sample to be purified is adjusted to the pH and ionic strength of the equilibration buffer. The sample is applied to the column and the column is washed after the load to remove unbound material. A salt, such as sodium chloride or potassium chloride, can be used to elute the α-Gal A from the column. Alternatively, the α-Gal A can be eluted from the column with a buffer of higher pH or a combination of higher salt concentration and higher pH. The α-Gal A can also be made to flow through the column during loading by increasing the salt concentration in the equilibration buffer and in the sample load, by running the column at a higher pH, or by a combination of both increased salt and higher pH.

Another step of purification can use a Q Sepharose® 6 Fast Flow for the purification of α-Gal A. Q Sepharose® 6 Fast Flow is a relatively strong anion exchange resin. A weaker anion exchange resin such as DEAE Sepharose® Fast Flow (Pharmacia) or Macro-Prep® DEAB (Bio-Rad) can also be used to purify α-Gal A. The column is equilibrated in a buffer, e.g., 10 mM sodium phosphate, pH 6. The pH of the sample is adjusted to pH 6, and low ionic strength is obtained by dilution or diafiltration of the sample. The sample is applied to the column under conditions that bind α-Gal A. The column is washed with equilibration buffer to remove unbound material. The α-Gal A is eluted with application of salt, e.g., sodium chloride or potassium chloride, or application of a lower pH buffer, or a combination of increased salt and lower pH. The α-Gal A can also be made to flow through the column during loading by increasing the salt concentration in the load or by running the column at a lower pH, or by a combination of both increased salt and lower pH.

Another step of purification can use a Superdex® 200 (Pharmacia) size exclusion chromatography for purification of α-Gal A. Other size exclusion chromatography resins such as Sephacryl® S-200 HR or Bio-Gel® A-1.5 m can also be used to purify α-Gal A. The in some embodiments the buffer for size exclusion chromatography is 25 mm sodium phosphate, pH 6.0, containing 0.15 M sodium chloride. Other formulation-compatible buffers can also be used, e.g., 10 mM sodium or potassium citrate. The pH of the buffer can be between pH 5 and pH 7 and should contain a salt, e.g., sodium chloride or a mixture of sodium chloride and potassium chloride.

Another step of purification can use a chromatofocusing resin such as Polybuffer Exchanger PBE 94 (Pharmacia) to purify α-Gal A. The column is equilibrated at relatively high pH (e.g., pH 7 or above), the pH of the sample to be purified is adjusted to the same pH, and the sample is applied to the column. Proteins are eluted with a decreasing pH gradient to a pH such as pH 4, using a buffer system, e.g., Polybuffer 74 (Pharmacia), which had been adjusted to pH 4.

Alternatively, immunoaffinity chromatography can be used to purify α-Gal A. An appropriate polyclonal or monoclonal antibody to α-Gal A (generated by immunization with α-Gal A or with a peptide derived from the α-Gal A sequence using standard techniques) can be immobilized on an activated coupling resin, e.g., NHS-activated Sepharose® 4 Fast low (Pharmacia) or CNBr-activated Sepharose®. 4 Fast Flow (Pharmacia). The sample to be purified can be applied to the immobilized antibody column at about pH 6 or pH 7. The column is washed to remove unbound material. α-Gal A is eluted from the column with typical reagents utilized for affinity column elution such as low pH, e.g., pH 3, denaturant, e.g., guainidinie HCl or thiocyanate, or organic solvent, e.g., 50% propylene glycol in a pH 6 buffer. The purification procedure can also use a metal chelate affinity resin, e.g., Chelating Sepharose® Fast Flow (Pharmacia), to purify α-Gal A. The column is pre-charged with metal ions, e.g., Cu⁺², Zn⁺², Ca⁺², Mg⁺² or Cd⁺². The sample to be purified is applied to the column at an appropriate pH, e.g., pH 6 to 7.5, and the column is washed to remove unbound proteins. The bound proteins are eluted by competitive elution with imidazole or histidine or by lowering the pH using sodium citrate or sodium acetate to a pH less than 6, or by introducing chelating agents, such as EDTA or EGTA.

### Dosages for Administration of α-Gal A Preparation

The α-Gal A preparations described herein exhibit a desirable circulatory half-life and tissue distribution, e.g., to capillary endothelial cells, renal glomerular epithelial cells (podocytes) and glomerular mesangial cells, and/or cardiac myocytes. Such preparations can be administered in relatively low dosages. For example, the unit dose of administration can be between 0.05-2.0 mg per kilogram body weight (mg/kg). For example, the unit dose can be between 0.05 and 1.0 mg, between 0.5 and 0.5 mg/kg, or between 0.5 and 0.3 mg/kg. Unit doses between 0.05 and 0.29 mg/kg are used in some embodiments, e.g., a unit dose of about 0.05, 0.1, 0.15, 0.2, 0.25, mg/kg. Assuming a specific activity of the α-Gal A preparation of between 2 and 4.5×10⁶ U/mg, these values correspond to about 0.1×10⁶ to 1.3×10⁶ U/kg. In certain embodiments, a unit dose saturates liver uptake of the α-Gal A.

Regularly repeated doses of the protein are necessary over a period of time, e.g., for a period of several months or 1, 2, 3 years or longer, even for the life of the patient. However, the desirable circulatory half-life and tissue distribution of the α-Gal A preparations described herein allow for the administration of the unit dose to a patient at intervals. For example, a unit dose can be administered at a frequency of about every day, every two days, every three days, every four days, every five days, or every six days.

As will be understood by those of ordinary skill in the art, the concentration of a drug in subject administered a single dose of a drug begins low at the time of administration, rises to a peak concentration over time, and then declines in concentration over time. In some embodiments, the dose of α-Gal A is sufficient to result in a peak concentration of α-Gal A in the kidney of the subject receiving the dose within about 45, 40, 35, 30, 25, or fewer hours after administration of the dose to the subject. Thus, the level in the kidney declines from the peak level within 45 or fewer hours after administration. In some embodiments the dose administered to a subject is a dose sufficient to result in a peak concentration followed by onset of a decline in concentration within about 24 hours or less after administration of the dose to the subject. Those of ordinary skill in the art will be able to determine peak concentrations of α-Gal A in the kidney using standard methods. For example, peak concentrations may be determined via imaging methods, use of detectable labels, etc.

During the time of therapy, a patient can be monitored clinically to evaluate the status of his or her disease. Clinical improvement measured by, for example, improvement in renal or cardiac function or patient's overall well being (e.g., pain), and laboratory improvement measured by, for example, reductions in urine, plasma, or tissue Gb3 levels, may be used to assess the patient's health status. In the event that clinical improvement is observed after a treatment and monitoring period, the frequency of α-Gal A administration may be reduced. For example, a patient receiving injections of α-Gal A preparation every four days may change to administration every six days; a patient receiving injections of an α-Gal A preparation every three days may switch to administration every five days; a patient receiving injections of an α-Gal A preparation every two days may switch to injections every three days, etc. Following such a change in dosing frequency, the patient should be monitored for another period of time, e.g., several years, e.g., a three year period, in order to assess Fabry disease-related clinical and laboratory measures. In some embodiments, the administered-dose does not change if a change in dosing frequency is made. This ensures that certain pharmacokinetic parameters (e.g. maximal plasma concentration [Cₘₐₓ], time to maximal plasma concentration [tₘₐₓ], plasma, half-life [t_{1/2}], and exposure as measured by area under the curve [AUC]) remain relatively constant following each administered dose. Maintenance of these pharmacokinetic parameters will result in relatively constant levels of receptor-mediated uptake of α-Gal A into tissues as dose frequencies change. In some embodiments of the invention, after a period of administering α-Gal A every two, three, four, five, or six days a subject may be switched to an even less frequent dosing interval for a period of time. Thus, in some embodiments, a subject may be treated with alternating shorter and longer administration intervals, wherein the shorter intervals include administration of α-Gal A every two, three, four, five, or six days.

In some embodiments, a patient is clinically evaluated between doses and a determination can be made upon evaluation as to the timing of the next dose. A patient with atypical variant of Fabry disease, e.g., exhibiting predominantly cardiovascular abnormalities or renal involvement, can be treated with these same dosage regiments as described herein. The dose is adjusted as needed. For example, a-patient with the cardiac variant phenotype who is treated with α-Gal A enzyme replacement therapy will have a change in the composition of their heart and improved cardiac function following therapy. This change can be measured with standard echocardiography which is able to detect increased left ventricular wall thickness in patients with Fabry disease (Goldman et al., J Am Coll Cardiol 7: 1157-1161 (1986)). Serial echocardiographic measurements of left ventricular-wall thickness can be conducted during therapy, and a decrease in ventricular wall size is indicative of a therapeutic response. Patients undergoing α-Gal A enzyme replacement therapy can also be followed with cardiac magnetic resonance imaging (MRI). MRI has the capability to assess the relative composition of a given tissue. For example, cardiac MRI in patients with Fabry disease reveals deposited lipid within the myocardium compared with control patients (Matsui et al., Ani Heart J 117: 472-474. (1989)). Serial cardiac MRI evaluations in a patient undergoing enzyme replacement therapy can reveal a change in the lipid deposition within a patient's heart. Patients with the renal variant phenotype can also benefit from α-Gal A enzyme replacement therapy. The effect of therapy can be measured by standard tests of renal function, such as 24-hour urine protein level, creatinine clearance, and glomerular filtration rate.

In some aspects of the invention, the effect of therapy can be measured by assessing mesangial widening in kidney glomeruli using art-known methods and an effective therapy may include a decrease in the fraction of glomeruli with mesangial widening. In certain aspects of the invention, the effect of therapy can be measured by assessing whether a subject's glomeruli are normal or if they exhibit abnormal characteristics, such as mesangial widening, etc.

The result of administration of an α-Gal A preparation of the invention may be a statistically significant result, may be a result sufficient to relieve symptoms in a subject, a result sufficient to result in a physiological change in the subject such that it is a regression of symptoms or the disease being treated, etc. As used herein the term “subject”, “individual”, and “patient” are used interchangeably and mean any mammal that may be in need of treatment with an α-Gal A formulation or preparation of the invention. Subjects include but are not limited to: humans, non-human primates, cats, dogs, sheep, pigs, horses, cows, rodents such as mice, rats, etc.

α-Gal A preparations and formulations of the invention are administered in effective amounts. An effective amount is that amount of a pharmaceutical preparation that alone, or together with further doses, stimulates the desired response. In the case of treating a disorder or condition, for example Fabry disease, that is associated with abnormal α-Galactosidase A levels or activity, a desired response is reducing the onset, stage, or progression of the abnormal α-Galactosidase activity or function and associated effects. This may involve only slowing the progression of the disease and/or damage temporarily, although more preferably, it involves halting the progression of the disease and/or damage permanently. An effective amount for treating Fabry disease may be that amount that alters increases α-Gal A activity in a subject with Fabry disease, with respect to that amount that would occur in the absence of amount of the administered α-Gal A preparation or formulation of the invention.

### Pharmaceutical Compositions

The α-Gal A preparations described herein are substantially free of non-α-Gal A proteins, such as albumin, non-α-Gal A proteins produced by the host cell, or proteins isolated from animal tissue or fluid. The preparation, in some embodiments, comprises part of an aqueous or physiologically compatible fluid suspension or solution. The carrier or vehicle is physiologically compatible so that, in addition to delivery of the desired preparation to the patient, it does not otherwise adversely affect the patient's electrolyte and/or volume balance. Useful solutions for parenteral administration may be prepared by any of the methods well known in the pharmaceutical art (See, e.g., REMINGTON′S PHARMACEUTICAL SCIENCES Gennaro, A., ed., Mack Pub., 1990). Non-parenteral formulations, such as suppositories and oral formulations, can also be used.

### α-Gal A Formulations

Formulations and compositions of α-Gal A preparations may be optimized for pH, protein concentration, carbohydrate content, surfactant inclusion, etc. Such parameters may be adjusted and compositions readily examined for optimization.

In some embodiments of the invention, an α-Gal A formulation or composition contains an excipient. Pharmaceutically acceptable excipients for α-Gal A which may be included in the formulation or composition are buffers such as citrate buffer, phosphate buffer, acetate buffer, and bicarbonate buffer, amino acids, urea, alcohols, ascorbic acid, phospholipids; proteins, such as serum albumin, collagen, and gelatin; salts such as EDTA or EGTA, and sodium chloride; liposomes; polyvinylpyrollidone; sugars, such as dextran, mannitol, sorbitol, and glycerol; propylene glycol and polyethylene glycol (PEG); glycerol; glycine or other amino acids; and lipids. In some embodiments, excipients are mannitol, sorbitol, glycerol, amino acids, lipids, EDTA, EGTA, sodium chloride, polyethylene glycol, polyvinylpyrollidone, dextran, or combinations of any of these excipients.

Carbohydrates

In some embodiments, a carbohydrate is included in the composition or formulation. E.g., a carbohydrate can cause the α-Gal A protein to be more compact, and for example, bury or otherwise hinder access to a moiety of the α-Gal A protein. This can increase protein stability, e.g., by reducing protein aggregation.

Carbohydrates include non-reducing sugars, e.g., non-reducing disaccharides, e.g., sucrose or trehalose, which are suitable for this purpose. The level of sugar in the composition can be critical. A sugar content of about 1 to about 40%, e.g., about 2 to about 30%, e.g., about 2 to about 10%, e.g., about 5%, weight per volume (w/v) is suitable, e.g., for use with α-Gal A. In some embodiments, a sugar content of about 3 to about 5% is suitable. In some embodiments, a sugar content from about 2% to about 10% sucrose is suitable. In some embodiments, a sugar content of about 5% sucrose is suitable.

One can test a candidate substance, e.g., a carbohydrate, for increasing α-Gal A protein stability. The stability of the α-Gal A composition containing the candidate substance, measured, e.g., as a percent aggregation or degradation, at a predetermined time is compared with one or more standards. For example, a suitable standard would be a composition similar to the test conditions except that a substance is not added to the composition. The stabilities of the treated (containing the substance) and untreated (lacking a substance) compositions are compared. Suitability can be shown by the test treatment increasing stability as compared with this standard. Another standard can be a composition similar to the test composition except that in place of the candidate substance, a substance described herein, for example, sucrose, is added to the composition. Suitability can be shown by the candidate substance having comparable or better effects on stability than a substance described herein. If the candidate substance increases stability of the composition as compared to one of the standards, the concentration of the candidate substance can be refined. For example, the concentration can be increased or decreased over a range of values and compared to the standard and to the other concentrations being tested to determine which concentration causes the greatest increase in stability.

α-Gal A protein stability can be measured, e.g., by measuring protein aggregation or protein degradation. α-Gal A protein aggregation can be determined, e.g., by size exclusion chromatography, non-denaturing PAGE, or other methods for determining size, etc. Protein degradation can be determined, e.g., by reverse phase HPLC, non-denaturing PAGE, ion-exchange chromatography, peptide mapping, or similar methods.

In some embodiments, a carbohydrate is trehalose or sucrose. Other substances that can used to stabilize the α-Gal A protein include, maltose, raffinose, glucose, sorbitol, lactose, arabinose; polyols such as mannitol, glycerol, and xylitol; amino acids such as glycine, arginine, lysine, histidine, alanine, methionine, and leucine; and polymers such as PEG, poloxomers, dextran, polypropylene glycol, polysaccharides, methylcellulose, sodium carboxymethyl cellulose, polyvinyl pyrrolidone (PVP), hydrolyzed gelatin, and human albumin.

In some embodiments, an α-Gal A formulation or composition comprises a non-ionic detergent. In some embodiments, non-ionic detergents include Polysorbate 20, Polysorbate 80, Triton X-100™, Triton X-114™, Nonidet P-40™, Octyl α-glucoside, Octyl β-glucoside, Brij. 35, Pluronic™, Poloxamer 188 (a.k.a. Poloxalkol) and Tween 20™. In certain embodiments, the non-ionic detergent comprises Polysorbate 20 or Polysorbate 80.

Surfactants

A surfactant can be added to the liquid protein (e.g., α-Gal A) composition or formulation. In some embodiments, this can increase protein stability, e.g., reduce protein degradation, e.g., due to air/liquid interface upon shaking/shipment. A surfactant that increases protein stability, e.g., does not cause protein degradation, in the liquid composition is selected. A surfactant suitable for use is e.g., poloxamer 188, e.g., PLURONIC® F68.

Ideally, a surfactant selected for use in the protein compositions described herein is one that is not modified, e.g., cleaved, by the protein.

For example, one can test a candidate surfactant by providing a composition or formulation containing α-Gal A that is adjust to pH 6.0, and adding the candidate surfactant. The stability of the α-Gal A composition or formulation containing the candidate surfactant, measured, e.g., as a percent aggregation or degradation, at a predetermined time is compared with one or more standards. For example, a suitable standard would be a composition similar to the test conditions except that a surfactant is not added to the composition. The stabilities of the treated (containing the surfactant) and untreated (lacking a surfactant) compositions are compared in conditions simulating “real world” scenarios, e.g., shipping. Suitability can be shown by the test treatment increasing stability as compared with this standard. Another standard can be a composition similar to the test composition except that in place of the candidate surfactant, a surfactant described herein, for example, poloxamer 188, is added to the composition. Suitability can be shown by the candidate surfactant having comparable or better effects on stability than a surfactant described herein. If the candidate surfactant increases stability of the composition as compared to one of the standards, the concentration of the candidate surfactant can be refined. For example, the concentration can be increased or decreased over a range of values and compared to the standard and to the other concentrations being tested to determine which concentration causes the greatest increase in stability.

Protein stability can be measured, e.g., by measuring protein aggregation or protein degradation. Protein aggregation can be determined, e.g., by size exclusion chromatography, non-denaturing PAGE, or other methods for determining size, etc. Protein degradation can be determined, e.g., by reverse phase HPLC, non-denaturing PAGE, ion-exchange chromatography, peptide mapping, or similar methods.

In some embodiments, a formulation of the invention includes a surfactant. In certain embodiments, the surfactant is poloxamer 188. The percentage of poloxamer 188 or other surfactant compound may be in the range from about 0.05 to 0.5% w/v. In some embodiments, the percentage of poloxamer 188 or other surfactant is 0.05% w/v.

Anti-Micobial Agents

An anti-microbial agent can be added to the liquid protein (e.g., α-Gal A) composition. In some embodiments, this can increase protein stability and preparation stability, e.g., reduce protein degradation, reduction of contamination. An anti-microbial agent that increases protein stability, e.g., does not cause protein degradation, and/or reduces contamination in the composition is selected. An antimicrobial suitable for use is e.g., Benzyl Alcohol, Phenol, m-crescol, or parabens. In some embodiments, benzyl alcohol is included in a formulation of the invention.

One can test a candidate anti-microbial by providing a composition containing α-Gal A that is adjust to pH 6.0, and adding a candidate anti-microbial. The stability of the α-Gal A composition containing the candidate anti-microbial, measured, e.g., as a percent aggregation or degradation, at a predetermined time is compared with one or more standards. For example, a suitable standard would be a composition similar to the test conditions except that an anti-microbial is not added to the composition. The stabilities of the treated (containing the anti-microbial) and untreated (lacking the anti-microbial) compositions are compared in conditions simulating “real world” scenarios, e.g., over time, etc. Suitability can be shown by the test treatment increasing stability as compared with this standard. Another standard can be a composition similar to the test composition except that in place of the candidate anti-microbial, an anti-microbial described herein, for example, benzyl alcohol, is added to the composition. Suitability can be shown by the candidate anti-microbial having comparable or better effects on stability than an anti-microbial described herein. If the candidate anti-microbial increases stability of the composition as compared to one of the standards, the concentration of the candidate anti-microbial can be refined. For example, the concentration can be increased or decreased over a range of values and compared to the standard and to the other concentrations being tested to determine which concentration causes the greatest increase in stability.

Stability of the composition (e.g., protein stability and/or reduction in contamination) can be measured, e.g., by measuring protein aggregation or protein degradation or contaminant growth or presence. Protein aggregation can be determined, e.g., by size exclusion chromatography, non-denaturing PAGE, or other methods for determining size, etc. Protein degradation can be determined, e.g., by reverse phase HPLC, non-denaturing PAGE, ion-exchange chromatography, peptide mapping, or similar methods.

pH

In some embodiments, a formulation comprises phosphate-buffered saline, e.g., at pH 6. Buffer systems for use with α-Gal A preparations include citrate; acetate; bicarbonate; and phosphate buffers (all available from Sigma). Phosphate buffer is include in some embodiments. In certain embodiments, a pH range for α-Gal A preparations is pH 4.5-7.4.

pH can be important in achieving an optimized protein composition, e.g., a liquid protein composition with increased stability. pH can work by affecting the conformation and/or aggregation and/or degradation and/or the reactivity of the protein. For example, at a higher pH, O₂ can be more reactive. In compositions of the invention, the pH may be less than 7.0. The pH may be in the range of about 4.5 to about 6.5, the range of about 5.0 to about 6.5, may be about 6.0. With some proteins, aggregation can reach undesirable levels above pH 7.0 and degradation (e.g., fragmentation) can reach undesirable levels under pH 4.5 or 5.0, or above pH 6.5 or 7.0. In some embodiments, the pH is 6.0.

One can test a candidate pH by providing a composition containing α-Gal A and adjusting the composition to a candidate pH. The stability of the α-Gal A composition at the candidate pH, measured, e.g., as a percent aggregation or degradation, at a predetermined time is compared with one or more standards. For example, a suitable standard would be a composition similar to the test conditions except that the pH of the composition is not adjusted. The stabilities of the treated (the composition adjusted to the candidate pH) and untreated (the pH is not adjusted) compositions are compared. Suitability can be shown by the test treatment increasing stability as compared with this standard. Another standard can be a composition similar to the test composition except that in place of the candidate pH, the composition has a pH described herein, for example, pH 6.0. Suitability can be shown by the composition at the candidate pH having comparable or better effects on stability than the composition at pH 6.0.

Protein stability can be measured, e.g., by measuring protein aggregation or protein degradation. Protein aggregation can be determined, e.g., by size exclusion chromatography, non-denaturing PAGE, or other methods for determining size, etc. Protein degradation can be determined, e.g., by reverse phase HPLC, non-denaturing PAGE, ion-exchange chromatography, peptide mapping, or similar methods.

Buffers that can be used to adjust the pH of a protein composition include: histidine, citrate, phosphate, glycine, succinate, acetate, glutamate, Tris, tartrate, aspartate, maleate, and lactate. In some embodiments, the buffer is citrate. In some embodiments from about 5 mM to 10 mM citrate buffer may be included in a formulation of the invention. In some embodiments a formulation of the invention includes 5 mM citrate buffer.

Protein Concentration

A preferred protein (e.g., α-Gal A) concentration can be between about 0.1 to about 60 mg/ml, about 1 to about 60 mg/ml, about 5 to about 40 mg/ml, about 20 to about 35 mg/ml, or about 30 mg/ml.

One can test for a suitable protein concentration by providing a composition that includes α-Gal A, adjusting the pH to 6.0, adjusting the α-Gal A to a candidate concentration. The stability of the α-Gal A composition at the candidate concentration, measured, e.g., as a percent aggregation or degradation, at a predetermined time is compared with one or more standards. For example, a suitable standard would be a composition similar to the test conditions except that the α-Gal A concentration is a concentration described herein, e.g., 20 mg/ml. The stabilities of the α-Gal A at each concentration are compared. Suitability can be shown by the candidate concentration having comparable or better effects on stability than a concentration described herein.

Protein stability can be measured, e.g., by measuring protein aggregation or protein degradation. Protein aggregation can be determined, e.g., by size exclusion chromatography, non-denaturing PAGE, or other methods for determining size, etc. Protein degradation can be determined, e.g., by reverse phase HPLC, non-denaturing PAGE, ion-exchange chromatography, peptide mapping, or similar methods.

Additional Excipients

A preparation of the invention may include an excipient such as propylene glycol and polyethylene glycol (PEG); glycerol; glycine, or other amino acids; and lipids. In some embodiments, a formulation of the invention includes up to 3% glycerol. In some embodiments, a formulation of the invention includes from 1 to 2.5% glycerol.

For lyophilization of α-Gal A preparations, the protein concentration can be 0.1-10 mg/mL or more. Bulking agents, such as glycine, mannitol, albumin, and dextran, can be added to the lyophilization mixture. In addition, possible cryoprotectants, such as disaccharides, amino acids, and PEG, can be added to the lyophilization mixture. Any of the buffers, excipients, and detergents listed above, can also be added.

Formulations for administration may include glycerol and other compositions of high viscosity to help maintain the agent at the desired locus. Biocompatible polymers, in some embodiments, bioresorbable, biocompatible polymers (including, e.g., hyaluronic acid, collagen, polybutyrate, lactide, and glycolide polymers and lactide/glycolide copolymers) may be useful excipients to control the release of the agent in vivo. Formulations for parenteral administration may include glycocholate for buccal administration, methoxysalicylate for rectal administration, or cutnic acid for vaginal administration. Suppositories for rectal administration may be prepared by mixing an α-Gal A preparation of the invention with a non-irritating excipient such as cocoa butter or other compositions that are solid at room temperature and liquid at body temperatures.

Formulations for inhalation administration may contain lactose or other excipients, or may be aqueous solutions which may contain polyoxyethylene-9-lauryl ether, glycocholate or deoxycocholate. In some embodiments, an inhalation aerosol is characterized by having particles of small mass density and large size. Particles with mass densities less than 0.4 gram per cubic centimeter and mean diameters exceeding 5 μm efficiently deliver inhaled therapeutics into the systemic circulation. Such particles are inspired deep into the lungs and escape the lungs' natural clearance mechanisms until the inhaled particles deliver their therapeutic payload. (Edwards et al., Science 276: 1868-1872 (1997)). α-Gal A preparations of the present invention can be administered in aerosolized form, for example by using methods of preparation and formulations as described in U.S. Pat. Nos. 5,654,007, 5,780,014, and 5,814,607, each incorporated herein by reference.

Formulation for intranasal administration may include oily solutions for administration in the form of nasal drops, or as a gel to be applied-intranasally.

Formulations for topical administration to the skin surface may be prepared by dispersing the α-Gal A preparation with a dermatological acceptable carrier such as, a lotion, cream, ointment, or soap. Particularly useful are carriers capable of forming a film or layer over the skin to localize application and inhibit removal. For topical administration to internal tissue surfaces, the α-Gal A preparation may be dispersed in a liquid tissue adhesive or other substance known to enhance adsorption to a tissue surface. For example, several mucosal adhesives and buccal tablets have been described for transmucosal drug delivery, such as in U.S. Pat. Nos. 4,740,365, 4,764,378, and 5,780,045, each incorporated herein by reference.

Hydroxypropylcellulose or fibrinogen/thrombin solutions may also be incorporated. Alternatively, tissue-coating solutions, such as pectin-containing formulations may be used. The preparations of the invention may be provided in containers suitable for maintaining sterility, protecting the activity of the active ingredients during proper distribution and storage, and providing convenient and effective accessibility of the preparation for administration to a patient. An injectable formulation of an α-Gal A preparation might be supplied in a stoppered vial suitable for withdrawal of the contents using a needle and syringe. The vial would be intended for either single use or multiple uses. The preparation can also be supplied as a prefilled syringe. In some instances, the contents would be supplied in liquid formulation, while in others they would be supplied in a dry or lyophilized state, which in some instances would require reconstitution with a standard or a supplied diluent to a liquid state. Where the preparation is supplied as a liquid for intravenous administration, it might be provided in a sterile bag or container suitable for connection to an intravenous administration line or catheter. In some embodiments, the preparations of the invention are supplied in either liquid or powdered formulations in devices which conveniently administer a predetermined dose of the preparation; examples of such devices include a needle less injector for either subcutaneous or intramuscular injection, and a metered aerosol delivery device. In other instances, the preparation may be supplied in a form suitable for sustained release, such as in a patch or dressing to be applied to the skin for transdermal administration, or via erodible devices for transmucosal administration. In instances where the preparation is orally administered in tablet or pill form, the preparation might be supplied in a bottle with a removable cover. The containers may be labeled with information such as the type of preparation, the name of the manufacturer or distributor, the indication, the suggested dosage, instructions for proper storage, or instructions for administration.

### Methods of Administration of α-Gal A Preparation

The α-Gal A preparations described herein may be administered by any route which is compatible with the α-Gal A preparation. The purified α-Gal A preparation can be administered to individuals who produce insufficient or defective α-Gal A protein or who may benefit from α-Gal A therapy. Therapeutic preparations of the present invention may be provided to an individual by any suitable means, directly (e.g., locally, as by injection, implantation, or topical administration to a tissue locus) or systemically (e.g., orally or parenterally).

In some embodiments, the route of administration is subcutaneous. Other routes of administration may be oral or parenteral, including intra-arterial, intraperitoneal, ophthalmic, intramuscular, buccal, rectal, vaginal, intraorbital, intracerebral, intradermal, intracranial, intraspinal, intraventricular, intrathecal, intracisternal, intracapsular, intrapulmonary, intranasal, transmucosal, transdermal, or via inhalation. Intrapulmonary delivery methods, apparatus and drug preparation are described, for example, in U.S. Pat. Nos. 5,785,049, 5,780,019, and 5,775,320, each incorporated herein by reference. In some embodiments, the method of intradermal delivery is by iontophoretic delivery via patches; one example of such delivery is taught in U.S. Pat. No. 5,843,015, which is incorporated herein by reference.

A particularly useful route of administration is by subcutaneous injection. An α-Gal A preparation of the present invention is formulated such that the total required dose may be administered in a single injection of one or two milliliters. In order to allow an injection volume of one or two milliliters, an α-Gal A preparation of the present invention may be formulated at a concentration in which the preferred dose is delivered in a volume of one to two milliliters or the α-Gal A preparation may be formulated in a lyophilized form, which is reconstituted in water or an appropriate physiologically compatible buffer prior to administration. Subcutaneous injections of α-Gal A preparations have the advantages of being convenient for the patient, in particular by allowing self-administration, while also resulting in a prolonged plasma half-life as compared to, for example, intravenous administration. A prolongation in plasma half-life results in maintenance of effective plasma α-Gal A levels over longer time periods, the benefit of which is to increase the exposure of clinically affected tissues to the injected α-Gal A and, as a result, may increase the uptake of α-Gal A into such tissues. This allows a more beneficial effect to the patient and/or a reduction in the frequency of administration. Furthermore, a variety of devices designed for patient convenience, such as refillable injection pens and needle-less injection devices, may be used with the α-Gal A preparations of the present invention as discussed herein.

Administration may be by periodic injections of a bolus of the preparation, or may be administered by intravenous or intraperitoneal administration from a reservoir which is external (e.g., an IV bag) or internal (e.g., a bioerodable implant, a bioartificial organ, or a population of implanted α-Gal A production cells). See, e.g., U.S. Pat. Nos. 4,407,957 and 5,798,113, each incorporated herein by reference. Intrapulmonary delivery methods and apparatus are described, for example, in U.S. Pat. Nos. 5,654,007, 5,780,014, and 5,814,607, each incorporated herein by reference. Other useful parenteral delivery systems include ethylene-vinyl acetate copolymer particles, osmotic pumps, implantable infusion systems, pump delivery, encapsulated cell delivery, liposomal delivery, needle-delivered injection, needle-less injection, nebulizer, aeorosolizer, electroporation, and transdermal patch. Needle-less injector devices are described in U.S. Pat. Nos. 5,879,327; 5,520,639; 5,846,233 and 5,704,911, the specifications of which are herein incorporated by reference. Any of the α-Gal A preparation described above can administered in these methods.

The route of administration and the amount of protein delivered can be determined by factors that are well within the ability of skilled artisans to assess. Furthermore, skilled artisans are aware that the route of administration and dosage of a therapeutic protein may be varied for a given patient until a therapeutic dosage level is obtained. All patents and publications cited in this specification are incorporated by reference.

### EXAMPLES SECTION

### Example 1

### Pharmacokinetics and Biodistribution of [¹²⁵1]-Replagal® in Jugular-Vein Cannulated Rats: Subcutaneous Versus Intravenous Delivery

In this study iodinated α-galactosidase A ([¹²⁵I-Replagal®) was administered either subcutaneously or intravenously to jugular-vein cannulated Sprague-Dawley rats to assess pharmacokinetics and biodistribution of the labeled protein. Serial blood samples and terminal tissue samples were analyzed by gamma counting for presence of Replagal® (test article).

### Major Objectives:

To characterize the serum pharmacokinetics of subcutaneous (SC) versus intravenous (ICV) Replagal® administration in jugular-vein cannulated Sprague-Dawley rats.

To evaluate the tissue biodistribution of subcutaneous (SC) versus intraveneous (IV) Replagal® administration in jugular-vein cannulated Sprague-Dawley rats.

### Experimental Design

### Overview

Replagal® is currently approved for use in European markets when administered as an intravenous infusion. The goal of these experiments was to assess the feasibility of delivering Replagal® via alternate routes, specifically, subcutaneously. Data from these studies, documented the bioavailability and tissue biodistribution of the Replagal® at three dose levels (5.0 mg/kg, 1.0 mg/kg, and 0.1 mg/kg) following either subcutaneous or intravenous administration. Intravenous injection and blood sampling was performed via the venous catheter. Summary pharmacokinetic (PK) and biodistribution data was analyzed and compared with previous studies.

### Introduction

Fabry disease is an X-linked disorder characterized by the absence of α-galactosidase A (aGalA), an enzyme required for the normal processing of glycosphingolipids in mammalian lysosomes. The loss of aGalA leads to accumulation of the neutral globotriaosylceramide (Gb₃), also known as ceramide trihexoside (CTH), within the heart, kidney, liver, and vascular endothelial cells. Renal and cardiac diseases are the most common cause of mortality and morbidity in Fabry patients (Thurberg et al, 2002; Tanaka et al., 2005). Hemizygous males, homozygous females, and some heterozygous females experience progressive organ dysfunction manifesting clinically as angiokeratomas, acroparathesis, stroke, cardiomyopathies, myocardial infarction and renal failure (Thurberg et al., 2002). The kidney is exceptionally susceptible to damage from Gb₃ deposition with several published reports of glycosphingolipid localized to the podocytes, vascular endothelial cells, and epithelial cells of the glomerulus. Loss of podocytes by apoptosis leads to glomerulosclerosis and drastically reduced kidney function. Affected individuals vary in disease progression and severity of symptoms. Historically, treatment options for Fabry patients were limited to symptomatic relief of renal and cardiovascular complications (Desnick et al., 2002). Attempts at more severe treatments, namely organ transplantation (Cho and Kopp, 2004; Sessa et al., 2002) and plasmapheresis (Winters et al., 2002), did not prove successful. Currently, two galactosidase drugs are approved in the European Union for treatment of Fabry disease via enzyme replacement therapy (ERT): agalsidase alfa (Replagal®, TKT/Shire) and agalsidase beta (Fabrazyme®, Genzyme). These protein based therapeutics deliver galactosidase activity to the lysosomes of affected organs in order to reduce the level of Gb₃ accumulation.

The experiments evaluated the equivalence of subcutaneous administration of Replagal, in this case iodinated Replagal®, to the current therapeutic standard, intravenous injection. The test material used herein was manufactured in roller bottles and passed clinical quality benchmarks.

### Materials and Methods

### Animals

Jugular vein cannulated (JVC) Sprague-Dawley rats were obtained from Taconic (Germantown, NY, USA) at 6-8 weeks of age. As described by the supplier, surgical modification involved the cannulation of the right common jugular vein with 0.023-inch (ID) polyethylene tubing equipped with a silicone rubber intravascular tip and secured with nylon sutures. The remaining 25 mm of cannula was passed beneath the clavicle and externalized between the scapulae using a small midline incision. An anchoring bead was secured along with the skin edges using stainless steel wound clips. A sterile, stainless steel pin sealed the cannulae and was removed for venous access. The average dead volume of the catheter was 30 μL. Animals were housed singly with free access to food and water before and during the experiment. Environmental enrichment was provided via food supplementation. Three rats out of 40 were sacrificed due to morbidity related to the indwelling catheter and wound clips. These studies complied with USDA regulations and the approved procedures outlined in the Shire Human Genetic Therapies Animal Care and Use Protocol, entitled “Injections of Therapeutic Proteins in Mice and Rats.”

### Test Article

Pre-filled vials of Replagal® (agalsidase alfa), Lot # FG923-004, were obtained from Shire Human Genetic Therapies (Cambridge, Mass.). The concentration was 1.0 mg protein per ml of storage buffer, with a total of 45 mg available for these experiments. The specific enzymatic activity of Lot # FG923-004 was reported at 3.2×10⁶ U/mg. Radiolabelled Replagal® was utilized as a tracer for these pharmacokinetic and biodistribution experiments. Iodination using the lactoperoxidase method (Parker, 1990; Marchalonis, 1969) was performed by PerkinElmer (Billerica, Mass., USA) with 500 μg of Replagal. The final iodinated product contained 100 μCi/mL or approximately 200,000 CPM/μL. Dosing solutions consisted of unlabeled Replagal® mixed with [¹²⁵I]-Replagal® for a target radioactivity of 2,000,000 CPM/animal. Both cold and iodinated products were stored at 4° C. until use.

### Animal Dosing Procedures

Intravenous dosing was performed using a 23-gauge 1″ aluminum hub blunt needle (Kendall Healthcare, Mansfield, MA) attached to a 1.0-ml slip-tip disposable syringe (BD Bioscience, Franklin Lakes, N.J.). Animals were passively restrained in plastic Decapicone® bags (Braintree Scientific, Braintree, Mass.), secured around the tail using 2″-binder clips. A small triangular opening was made in the plastic bag, through which the catheter was accessed. Immediately after dosing, the catheter was flushed with 0.1 ml of sterile saline to ensure the entire dose volume entered the vasculature. Intravenous dosing was well tolerated, with no obvious discomfort during or immediately following dose administration. Table 1 reports animal weight, dose volume, CPM/animal and circulating blood volume for all experimental animals used in this study.

### Serial Blood Collection

At each time point, the sterile pin sealing the external catheter was gently removed while the rat was restrained in a Decapicone®. A 22-gauge blunt needle attached to a 1.0-ml plastic disposable syringe was inserted into the open catheter and gentle pressure employed to remove 0.2 ml of blood. After sufficient sample was obtained, the plastic syringe was removed, leaving the needle in place, and the blood sample transferred to a 0.4-ml serum separator microtainer tube (BD Bioscience, Franklin Lakes, N.J.). A separate syringe was attached to the needle and the catheter was flushed with approximately 30 μL of sterile saline. The blunt needle was removed from the catheter while gently pressure was applied below the tip to prevent back flow of blood. The sterile pin was replaced to seal the catheter. Serial samples were obtained at 2, 5, 10, 15, 30, 60, 90, 120, and 180 min post-treatment. Blood samples were immediately centrifuged at 11,000 rpm (8,500×g) in a fixed-angle rotor for 2 min at room temperature. Serum was transferred to labeled 1.2-ml cryovials (Fisher Scientific, Chicago, Ill.) and stored at −80° C. until analysis. The mean total volume of blood removed was less than 10% of total circulating blood as shown below in Table 1.

TABLE 1
Calculation of % CBV (Circulating Blood Volume) Sampled

| | | | Range | |
|---|---|---|---|---|
| | Mean | SEM | Min | Max |
| Animal Weight (kg) | 0.324 | 0.06 | 0.242 | 0.440 |
| Circulating Blood Volume | 20.7 | 3.7 | 15.6 | 28.2 |
| (CBV) (mL) | | | | |
| Percent CBV Sampled | 8.87% | 0.29% | 6.4% | 11.6% |

### Biodistribution in Target Tissues

In order to assess the biodistribution of [125]-Replagal® several target tissues were sampled. A portion of the liver, spleen, both kidneys, and heart were removed for analysis. The thyroid was also harvested to assess uptake of radiolabelled iodine. Each tissue sample was harvested by blunt dissection and placed immediately in a 1.2-ml cryovial for storage. Filled vials were weighed prior to analysis. No further processing was performed on the tissue samples and the organs were submitted for gamma counting.

### Gamma Counting

The radioactivity of serum and tissue samples from rats injected with [¹²⁵I]-Replagal® was quantified using a Wallace WIZARD automatic gamma counter (PerkinElmer, Boston, Mass.). Tissue samples and 100 μL serum aliquots were thawed and transferred to 12×55 mm polycarbonate RIA (radioimmunoassay) tubes (PerkinElmer, Boston, Mass.). A pre-programmed protocol was utilized for the analysis. Briefly, the disintegrations per minute (DPM) of each sample were measured over 60 sec. The DPM was then converted to CPM using an internal efficiency algorithm. An aliquot of the dosing solution (100 μL) from each treatment group was analyzed along with the samples to ensure delivery of adequate and appropriate radioactivity for each animal. Blank samples consisted of 100 μL of control serum. All raw data was blank-corrected before further analysis.

### Data Analysis

Non-compartmental pharmacokinetic parameters were calculated using GraphPad Prism v.4.0 software (San Diego, Calif.) and MS 2003 Excel (Redmond, Wash.). Graphs of serum radioactivity-time curves were created in Prism. The elimination constant (kₑ) was obtained by transforming serum radioactivity using Y=Ln(Y) and plotting the results on a log-linear scale versus time. The slope of the best-fit first order line was employed to estimate kₑ. The y-intercept of the best-fit line represents the extrapolated serum radioactivity at time=0 (C₀). Area under the concentration-time curve (AUC) was calculated using standard equations provided in Prism software. Calculated PK parameters are summarized in Table 2. Total serum clearance (Cl), volume of distribution (V_{d}), and bioavailability (F) parameters were calculated as shown below using these equations:

t_{1/2}=ln(2)/kₑ=0.693/kₑ

V_{d}=AUC/C₀

Cl=(kₑ)*(V_{d})

F=(AUC_{sc}/AUCᵢᵥ)*100

TABLE 2

| | 5.0 mg/kg | | 1.0 mg/kg | | 0.1 mg/kg | |
|---|---|---|---|---|---|---|
| | Group A | Group B | Group C | Group D | Group E | Group F |
| Parameter | IV | SC | IV | SC | IV | SC |
| Cₘₐₓ | 45428 ± 16550 | 784 ± 51 | 22162 ± 4301 | 371 ± 106 | 34635 ± 7005 | 746 ± 80 |
| (CPM/mL) | | | | | | |
| tₘₐₓ | 2 | 180 | 2 | 15 | 2 | 120 |
| (min) | | | | | | |
| kₑ | 0.017 | −0.0018 | 0.016 | −0.0017 | 0.13 | −0.003 |
| t_{1/2} | 40.8 | ND | 43.6 | ND | 53 | ND |
| (min) | | | | | | |
| C₀ | 22697 | 582 | 5767 | 247 | 7555 | 388 |
| AUC | 1,020,000 | 126,125 | 276,041 | 53,875 | 418,408 | 104,032 |
| V_{d} | 44.9 | 217 | 47.8 | 218 | 55.4 | 268 |
| (mL) | | | | | | |
| Cl | 0.763 | −0.39 | 0.760 | −0.36 | 0.72 | −0.80 |
| (mL/min) | | | | | | |
| F | 100% | 12.4% | 100% | 19.5% | 100% | 24.8% |
| (%) | | | | | | |

### Results

### Intravenous [¹²⁵I]-Replagal® (Groups A, C, and E)

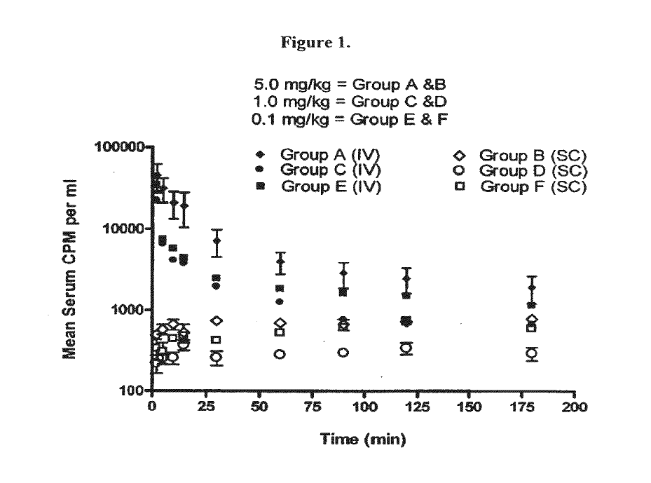

Radioactivity in serum (CPM/mL) across after intravenous (IV) test article exhibited a biphasic curve with rapid decline over followed by a gradual elimination phase (FIG. 1). Maximum radioactivity in serum (Cₘₐₓ) was achieved at 2 min (tₘₐₓ) post-administration for Groups A, C, and E. Only Groups A and C demonstrated a dose-response relationship, especially in terms of systemic exposure (Table 2). Non-linear regression curves of log-linear plots demonstrated poor fits with R² values of 0.824, 0.68, and 0.58 for Groups A, C, and E, respectively. Total clearance (Cl) and the elimination rate constants (kₑ) were very similar between groups (Table 2).

Tissue radioactivity following IV [¹²⁵I]-Replagal® localized primarily to the liver and spleen of treated animals. Although levels increased with increasing dose between Groups C and A, there was no overall dose-dependent trend in tissue radioactivity. Mean (±SEM) liver radioactivity was 29.2±1.9 CPM/mg, 11.2±3.1 CPM/mg, 21.0±0.7 CPM/mg for Groups A, C, and E, respectively. Spleen radioactivity was calculated at 7.4±0.2 CPM/mg, 0.6±0.2 CPM/mg, and 5.2±0.2 CPM/mg for Groups A, C, and E, respectively. Kidney levels of [¹²⁵I]-Replagal® were consistent between left and right organs and among dose groups, with combined mean values of 1.6±0.08 CPM/mg, 1.01±0.29 CPM/mg, and 1.05±0.12 CPM/mg for Groups A, C, and E, respectively. Mean cardiac tissue radioactivity was 1.07±0.17 CPM/mg (Group A), 0.53±0.23 CPM/mg (Group C), and 0.52±0.16 CPM/mg (Group E). Thyroid radioactivity was fairly low across all three IV groups, with mean levels of 0.67±0.19 (Group A), 2.35±0.17 (Group C), and 0.56±0.11 (Group E). Percent dose recovered was not calculated because only a few organs were sampled.

### Subcutaneous [¹²⁵I]-Replagal® (Groups B, D, and F)

Radioactivity in serum (CPM/mL) following subcutaneous (SC) test article was significantly lower than results obtained with direct intravenous injection of [¹²⁵I]-Replagal® (FIG. 1; Table 2). Time to (tₘₐₓ) maximal mean serum radioactivity (Cₘₐₓ) was 180 min to 784±51 CPM/ml (Group B), 15 min to 371±106 CPM/ml (Group D), and 120 min to 746±80 CPM/ml (Group F). Serum data was transformed using Y=Ln(Y) and plotted on a linear scale versus time. The first-order curve fit using non-linear regression was poor for all three

SC groups with R² values of 0.37 (Group B), 0.61 (Group D), and 0.63 (Group F). The best fit curve for the SC Ln(Y) vs. time data exhibited a positive slope, producing negative lc, values (Table 2), indicating net accumulation rather than elimination during this experiment. As such, Cl values were not determined for Groups B, D, and F.

Tissue radioactivity following SC [¹²⁵I]Replagal® was detected in all organs sampled. Mean (±SEM) liver radioactivity was 0.821±0.12 CPM/mg (Group B), 0.339±0.046 CPM/mg (Group D), and 0.694±0.095 CPM/mg (Group F). Spleen radioactivity was lower than dose-matched IV groups, with mean±SEM values of 0.718±0.453 CPM/mg (Group B), 0.218±0.046 CPM/mg (Group D), and 0.455±0.036 CPM/mg (Group F). Kidney radioactivity was well matched between left and right organs, with combined mean±SEM values of 0.82±0.035 CPM/mg, 0.56±0.02 CPM/mg, and 0.98±0.05 CPM/mg for Groups B, D, and F, respectively. Mean±SEM heart radioactivity was measured at 0.68±0.31 CPM/mg, 0.19±0.02, and 0.28±0.06 CPM/mg for Groups B, D, and F, respectively. Thyroid radioactivity was consistent between groups, with mean levels of 0.329±0.134 [This data represents n=4; one extreme statistical outier (>4 SD) was excluded from the mean analysis.] CPM/mg (Group B), 0.189±0.03 CPM/mg (Group D), and 0.265±0.06 (CPM/mg).

### Discussion

Pharmacokinetics of [¹²⁵I]-Replagal® in jugular-vein cannulated (JVC) rats demonstrated non-linear, dose-independent trends for both SC and IV routes. The test article was rapidly cleared from the serum of animals injected intravenously. The serum radioactivity did not fall below lower detection limits (LOD=50 CPM/ml) in IV treatment groups (FIG. 1), although a concentration plateau was achieved beginning at 60 to 90 min for most animals. An experiment with a longer duration may demonstrate complete elimination of test article from serum. Rats injected subcutaneously with test article failed to demonstrate net elimination from serum during this experiment. The elimination rate constants for all SC-treated groups (B, D, and F) were negative, indicating net accumulation of [¹²⁵I]-Replagal® in these animals over the course of the experiment (FIG. 1; Table 2).

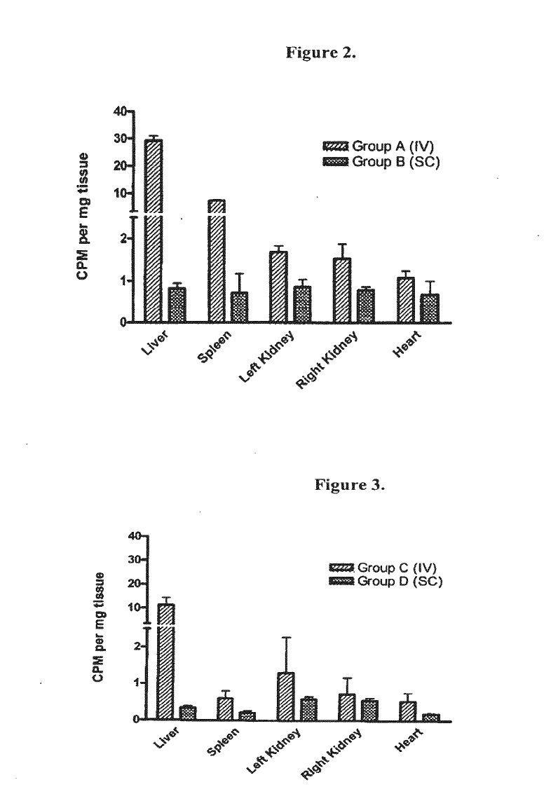

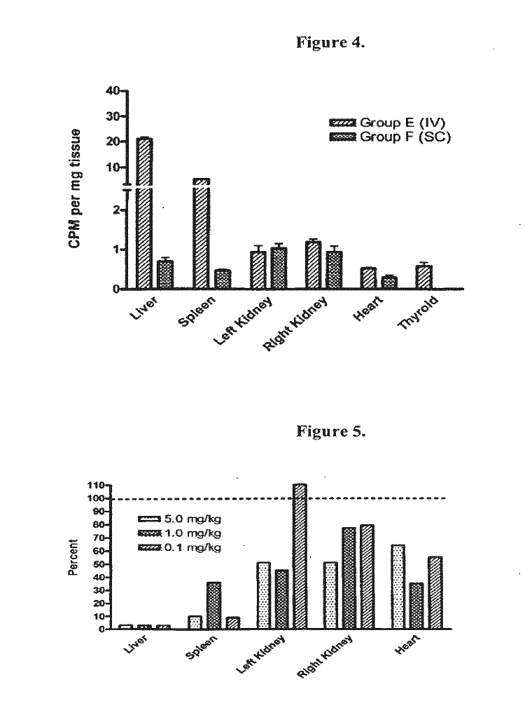

Although the overall tissue radioactivity was higher in IV-treated animals, when dose-matched the data suggest that SC treatment does deliver the iodinated test article to certain organs (FIGS. 2-4). In all IV-treated groups (A, C, and E), the liver was the primary reservoir of radioactivity, followed closely by the spleen and kidneys. Conversely, in SC groups (B, D, and F), the kidneys and heart contained the most radioactivity per mg. Thyroid radioactivity was low in all groups, suggesting non-specific tissue contamination during collection or previous disruption to the thyroid during surgical implantation of the jugular vein cannulae.

Serum and tissue radioactivity data from Group E (0.1 mg/kg IV) and Group F (0.1 mg/kg SC) did not reflect the dose administered. Radioactivity within Group C (1.0 mg/kg IV) should be approximately 10-fold higher than Group E, if a linear dose-response relationship exists in this model. Examination of the data reveals that rats in Group E had an overall higher amount of radioactivity in serum and tissue than Group C, although most PK parameters were similar between the two groups (Table 2). This may be due to the use of Replagal® vehicle (150 mM sodium chloride, 25 mM sodium phosphate monobasic, 0.02% Tween-20 in water) to dilute the test article prior to dosing. Groups E and F were the only treatment groups to receive vehicle-diluted material. The fixed initial concentration of the available test article (1 mg/ml) required delivery without dilution for Groups A/B (5.0 mg/kg), and C/D (1.0 mg/kg); this represents the only significant difference in terms of experimental procedure between treatment groups in this study.

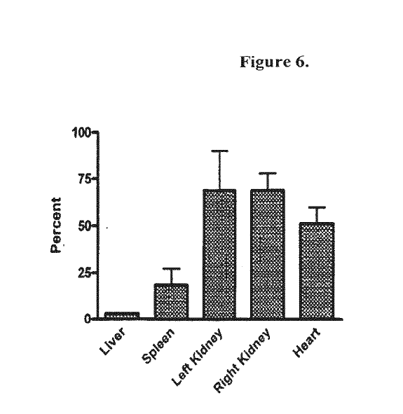

In summary, treatment with SC Replagal® appears to preferentially accumulate radioactivity into two tissue compartments: kidney and heart (FIGS. 5 and 6). Comparison of SC versus IV radioactivity in these two organs reveals that the SC route approaches, and in one instance exceeds, concentrations achieved with direct intravenous injection (FIG. 5). The overall trend between SC and IV is illustrated in FIG. 6, again emphasizing the accumulation of SC-derived radioactivity in kidneys and heart of treated rats. This trend is intriguing and would complement the therapeutic targets of Fabry disease in knockout mouse models and human patients, namely the renal and cardiac systems.

### References for Example 1

Cho M E and J B Kopp. 2004. Fabry disease in the era of enzyme replacement therapy: a renal perspective. Pediatr Nephrol, 19(6): 583-593.
Desnick R J, M Banikazemi, M Wasserstein. 2002. Enzyme replacement therapy for Fabry disease, an inherited nephropathy. Clinical Nephrology, 57(1): 1-8.
Sessa A, M Meroni, G Battini, A Maglio, M Nebuloni, A Tosoni, V Panichi, and B Bertagnolio. 2002. Renal transplantation in patients with Fabry disease. Nephron, 91(2): 348-351.
Tanaka M, T Ohashi, M Kobayashi, Y Eto, N Miyamura, K Nishida, E Araki, K Itoh, K Matsushita, M Hara, K Kuwahra, T Nakano, N Yasumoto, H Nonoguchi, and K Tomia. 2005.
Identification of Fabry's disease by the screening of α-galactosidase A activity in male and female patients. Clinical Nephrology, 64(4): 281-287.
Thurberg B L, H Rennke, R B Colvin, S Dikman, R E Gordon, A B Collins, R J Desnick, and M O'Callaghan. 2002. Globotriaosylceramide accumulation in the Fabry kidney is cleared from multiple cell types after enzyme replacement therapy. Kidney International, 62(6): 1933-1946.
Winters J L, A A Pineda, B C McLeod, and K M Grima. 2000. Therapeutic apheresis in renal and metabolic diseases. J Clin Apheresis, 15(1-2): 53-73.

### Example 2

Assessment of Replagal® Pharmacokinetics and Biodistribution Over 48 Hours after Subcutaneous Administration Experimental Design

The goal of these experiments was to assess the feasibility of delivering Replagal® subcutaneously. As described, the bioavailability and tissue biodistribution of Replagal®, administered subcutaneously as a single dose level (1.0 mg/kg) were assessed over a period of 48 hours after administration, a longer time period than in Example 1. Blood sampling was performed via an externalized venous catheter. Biodistribution of the test article in skin (peri-injection site and thigh skin), testes, kidneys, spleen, liver, heart, lungs, and thyroid was evaluated by gamma counting. Summary pharmacokinetic (PK) and biodistribution (BD) data were analyzed and compared with additional results, especially those described in Example 3, which is a parallel study examining PK and BD following intravenous injection under similar experimental conditions.

### Materials and Methods

### Animals

Jugular vein cannulated (JVC) Sprague-Dawley rats were obtained from Taconic (Germantown, NY, USA) at 6-8 weeks of age. As described by the supplier, surgical modification involved the cannulation of the right common jugular vein with 0.023-inch (ID) polyethylene tubing equipped with a silicone rubber intravascular tip and secured with nylon sutures. The remaining 25 mm of cannula was passed beneath the clavicle and externalized between the scapulae using a small midline incision. An anchoring bead was secured along with the skin edges using stainless steel wound clips. A sterile, stainless steel pin sealed the cannulae and was removed for venous access. The average dead volume of the catheter was 30 μL. Animals were housed singly with access to food and water ad libitum before and during the experiment. Environmental enrichment was provided via food supplementation and the use of Nylabones®. A total of twenty rats were selected for this experiment; 19 were injected SC with the iodinated test article as planned. The rat designated AS (5^{th} animal in Group A) had severe damage to the external catheter prior to dosing and was sacrificed as a control animal without injection. Tissue and blood were collected as outlined below for experimental animals. These studies complied with USDA regulations, Institutional Animal Care and Use Committee (IACUC) guidelines, and the IACUC approved procedures outlined in the institutional Animal Care and Use Protocol (ACUP) 46, entitled “Injections of Therapeutic Proteins in Mice and Rats.”

### Test Article

Pre-filled vials of Replagal® (agalsidase alfa), lot # FG923-004, were obtained from Shire Human Genetic Therapies (Cambridge, Mass.). The concentration was 1.0 mg protein per ml of storage buffer, with a total of 20 mg available for these experiments. The specific enzymatic activity of lot #FG923-004 was reported at 3.2×10⁶ U/mg. Radiolabelled ¹²⁵I-Replagal® was utilized as a tracer for these pharmacokinetic and biodistribution experiments. Iodination using the lactoperoxidase method (Parker, 1990; Marchalonis, 1969) was performed by PerkinElmer (Billerica, Mass., USA) with 500 μg of Replagal®. The final iodinated product contained 6.5 μCi/mL or approximately 14,000,000 CPM/mL. Dosing solutions consisted of unlabeled Replagal® mixed with ¹²⁵I-Replagal® for a final mean radioactivity of 3,597,370 CPM/animal. Both cold and iodinated products were stored at 4° C. until use. Dosing solution was equilibrated to room temperature for approximately 10 minutes prior to injection.

### Animal Dosing Procedures

Subcutaneous dosing was performed using a 25-gauge, ⅝″ stainless steel needle (BD Bioscience, Franklin Lakes, N.J.). Animals were restrained in plastic Decapicone® bags (Braintree Scientific, Braintree, Mass.), secured around the tail using 2″-binder clips. A small triangular opening was made in the plastic bag, through which the catheter was accessed. Baseline blood samples (0.25 mL) were removed via the catheter using a 23-gauge, ½″ aluminum hub blunt needle (Kendall Tyco Healthcare, Mansfield, Mass.) attached to a 1.0-ml plastic disposable syringe immediately prior to dosing. After baseline sampling, the opening on the plastic Decapicone was widened slightly and a fold of loose scapular skin externalized gently through the opening. The dosing needle tip was inserted beneath the skin with a quick motion and the plunger withdrawn slightly to check for blood. Absence of a blood flash indicated appropriate placement in the subcutaneous space and the dose volume was administered as a bolus (<30 sec). Subcutaneous dosing was well tolerated, with no obvious discomfort during or immediately following dose administration.

### Serial Blood Collection

At each time point, the sterile pin sealing the external catheter was gently removed while the rat was safely restrained in a Decapicone®. A 23-gauge blunt needle attached to a 1.0-ml plastic disposable syringe was inserted into the open catheter and gentle pressure employed to remove 0.25 mL of blood. After sufficient sample was obtained, the plastic syringe was removed, leaving the needle in place, and the blood sample transferred to a 0.4-ml serum separator microtainer tube (BD Bioscience, Franklin Lakes, N.J.). A separate syringe contained sterile saline was attached to the needle and the catheter was flushed with approximately 30 μL of the sterile solution to prevent clotting. The blunt needle was removed from the catheter while gentle pressure was applied below the tip to prevent backflow of blood. The sterile pin was replaced to seal the catheter. Serial samples were obtained on a pre-determined schedule as described below in Table 3. The mean total volume of blood removed was less than 10% of total circulating blood as shown below in Table 4. Blood samples were immediately centrifuged at 11,000 rpm (8,500×g) in a fixed-angle rotor for 2 min at room temperature. Serum was transferred to labeled 1.2-ml cryovials (Fisher Scientific, Chicago, Ill.) and stored at −80° C. until analysis.

TABLE 3
Blood Sampling Schedule by Group

| | | Group A | Group B | Group C | Group D |
|---|---|---|---|---|---|
| Time | | 2 hr Sac | 4 hr Sac | 24 hr Sac | 48 hr Sac |
| 0* | | X | X | X | X |
| 30 | min | X | X | | |
| 60 | min | X | X | X | X |
| 120 | min | X^{§} | X | X | X |
| 180 | min | | X | X | |
| 240 | min | | X^{§} | X | X |
| 480 | min | | | X | X |
| 24 | h | | | X^{§} | X |
| 36 | h | | | | X |
| 48 | h | | | | X^{§} |

TABLE 4
Calculation of % CBV (Circulating Blood Volumes) Sampled

| | | | | Range | |
|---|---|---|---|---|---|
| | Mean | | SEM | Min | Max |
| Animal weight (kg) | 0.266 | ± | 0.004 | 0.242 | 0.291 |
| Circulating Blood Volume | 17.002 | ± | 0.240 | 15.49 | 18.62 |
| (CBV (mL) | | | | | |
| Percent CBV Sampled per 24 hr | 10.3 | ± | 0.15 | 11.3 | 9.4 |

### Biodistribution in Target Tissues

In order to assess the biodistribution of ¹²⁵I-Replagal® several target tissues were sampled including injection site, thigh skin, testes (pooled), kidneys (pooled), spleen, liver, heart, lungs, and thyroid. Each tissue sample was harvested by blunt dissection and placed immediately in a 5-ml plastic conical tube (Fisher Scientific, Chicago, Ill.) for storage. The thyroid was harvested to assess uptake of radiolabelled iodine from systemic circulation not as a target tissue for Replagal®. The skin samples consisted of approximately 1 cm² portion of skin removed either from the injection site (scapular region; contained the actual site of skin puncture) or “distal” skin from the right thigh. Immediately after harvest, the entire organ (or pooled organs in the case of the kidneys and testes) was weighed; these values were recorded as “organ weight (g).” A portion of the testes and liver were removed for analysis as the entire organ would not fit inside a standard RIA (radioimmunoassay) tube. The remaining organs were counted intact after minimal deconstruction in order to fit the tissues inside RIA tubes. Control tissues were harvested from an untreated rat (B5) No further processing was performed on the tissue samples and the organs were submitted for gamma counting. Each tube containing tissue was weighed on an analytical balance, tared with an empty RIA tube, after gamma counting. This weight was recorded as “sample weight (g).” Differences between these two weight measurements were typically a few mg. Tissue samples that could not be counted immediately after harvest were held at 4° C. until analysis. The gamma counter is maintained at room temperature. Difference between “organ weight” (prior to counting) and “sample weight” (after counting) may be due to this temperature change and the resulting condensation within the RIA tubes. Random variation due to differences in balance accuracy and weighing technique also contributed to dissimilarity between the weight records for each tissue sample.

### Gamma Counting

The radioactivity of serum and tissue samples from rats injected with ¹²⁵I-Replagal® was quantified using a Wallace WIZARD automatic gamma counter (PerkinElmer, Boston, Mass.). Tissue samples and 100 μL serum aliquots were thawed and transferred to 12×55 mm polycarbonate RIA (radioimmunoassay) tubes (PerkinElmer, Boston, Mass.). A pre-programmed protocol was utilized for the analysis. Briefly, the disintegrations per minute (DPM) of each sample were measured over 60 sec. The DPM was then converted to CPM using an internal efficiency algorithm. An aliquot of the dosing solution (100 μL) from each treatment group was analyzed along with the samples to ensure delivery of adequate and appropriate radioactivity for each animal. “Blank” samples consisted of 100 μL of baseline serum or tissue samples from untreated rats. All raw data was corrected for background CPM during analysis.

### Data Analysis

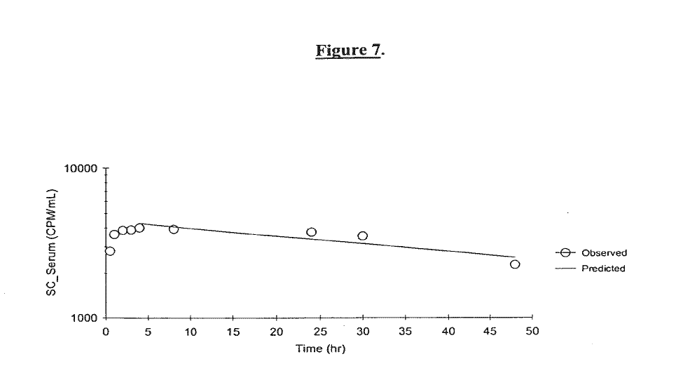

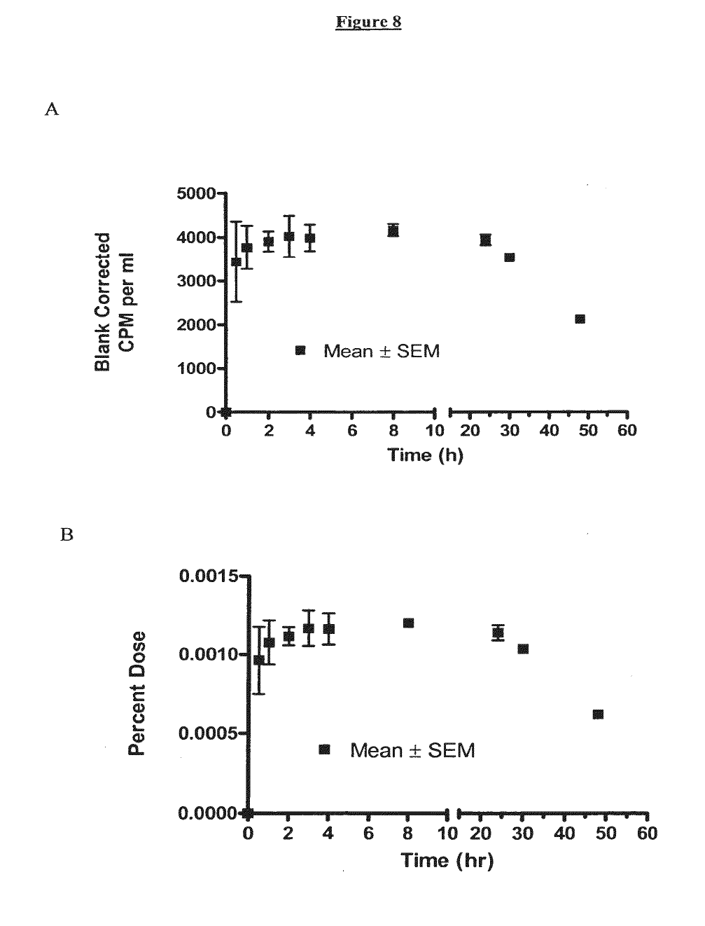

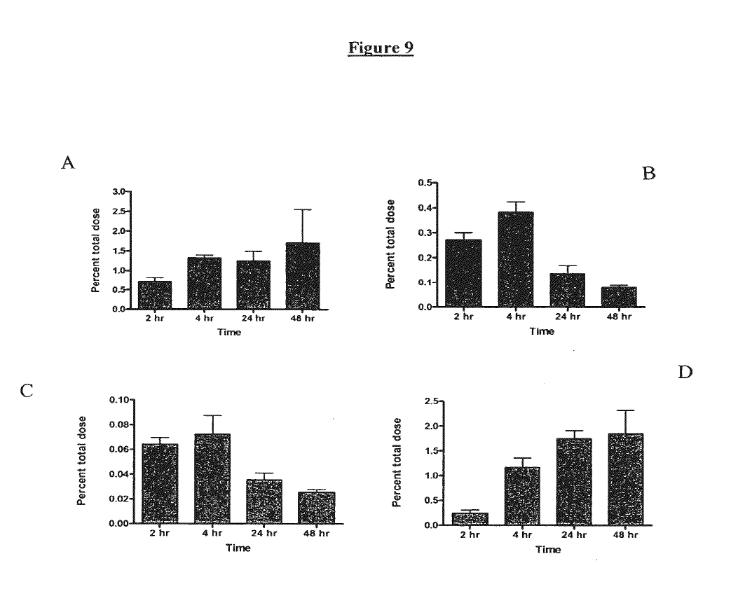

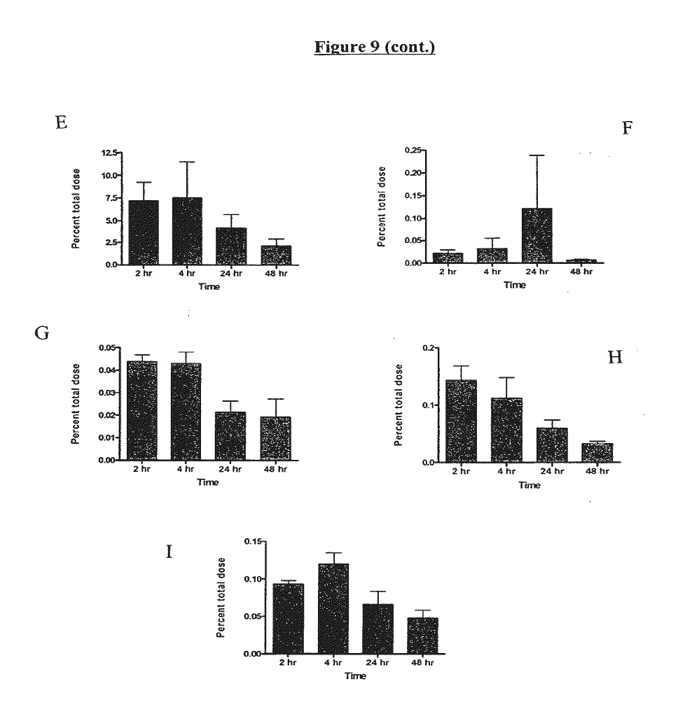

Raw data was tabulated in Microsoft Excel (v. 2003, Redmond, Wash.). Noncompartmental analysis of serum radioactivity was performed using WinNonLin Professional v. 5.0.1 (Pharsight, Mountain View, Calif.). A log-linear chart depicting the analyzed data is included in FIG. 7. The slope of the best-fit (lambda z) line was used to estimate an elimination half-life. Calculated PK parameters are summarized in Table 5. Summary charts for FIGS. 8 and 9 were created using Prism v.4 (GraphPad, San Diego, Calif.).

TABLE 5
Summary of Noncompartmental Pharmacokinetic Parameters

| | | Single SC | |
|---|---|---|---|
| Parameter | Units | Injection | Data Source |
| Cmax | CPM/ml | 4003 ± 281 | observed |
| tₘₐₓ | hr | 4 | observed |
| λ_{z} t_{1/2} | hr | 58.2 | observed |
| AUC_{inf} | hr · CPM/ml | 356763 | extrapolated from |
| | | | observed data |
| Cl | CPM/hr · CPM/mL | 92.2 | predicted from |
| | | | observed data |
| V_{d} | mL | 7750 | predicted from |
| | | | observed data |
| MRT_{inf} | hr | 84 | predicted from |
| | | | observed data |

### Results

### Serum Pharmacokinetics

Data for serum radioactivity were expressed two ways: a) CPM per mL, and b) percent of total dose. The latter method corrects for differences in dose radioactivity and allows direct interstudy comparison. All data analyzed for serum noncompartmental PK were expressed as CPM per mL. The concentration-time curves of serum radioactivity following subcutaneous (SC) ¹²⁵I-Replagal® exhibited distinct, albeit gradual, absorption and elimination phases FIG. 7. The lambda z (elimination) half-life was calculated at 58 hr. Maximal serum radioactivity of 4003±281 CPM/mL was achieved 4 hr after injection. The area under the concentration-time curve (AUC) was extrapolated to infinity and returned a value of 356,763. The predicted rate of total clearance was 92.2 CPM per mL/hr. Calculated PK parameters are summarized in Table 5.

### Tissue Biodistribution

Similar to the serum data presented above, tissue radioactivity was calculated using several methods: a) CPM per mg, b) total organ CPM, and c) percent total dose (derived from total organ CPM). In this report, percent total dose is utilized primarily to allow for direct comparison with other studies. FIG. 9 depicts individual graphs for all harvested tissues (injection site, distal skin, pooled testes, pooled kidneys, spleen, liver, heart, lungs, and thyroid) expressed as percent total dose versus time. Tissue half-life was calculated using the best-fit first order curve matched to log transformed data. Table 6 summarizes biodistribution data gathered in this study. Briefly, radioactivity in the kidneys reaches a maximum at 4 hr (Cₘₐₓ=0.4±0.04% total dose) whereas radioactivity in cardiac tissue peaked at 24 hr (Cₘₐₓ=0.08±0.024% total dose). Both the liver and the thyroid exhibited accumulation of radioactivity during this study as evidenced by the positive slope on the log-linear concentration vs. time curve.

TABLE 6
Summary of calculated tissue radioactivity biodistribution parameters.

| | CPM per g tissue | | | Percent Dose Administered | | |
|---|---|---|---|---|---|---|
| | Cₘₐₓ | tₘₐₓ | t_{1/2} | Cₘₐₓ | tₘₐₓ | t_{1/2} |
| Organ | (CPM) | (hr) | (hr) | (% Dose) | (hr) | (hr) |
| Injection Site | 2.03 × 10⁶ ± 1.02 × 10⁵ | 4 | 20 | 7.8 ± 2.4 | 2 | 30 |
| Skin | 9951 ± 6122 | 4 | 19 | 0.12 ± 0.12 | 24 | 15 |
| Testes | 1596 ± 364 | 2 | 21 | 0.14 ± 0.03 | 2 | 22 |
| Kidneys | 3631 ± 307 | 4 | 21 | 0.4 ± 0.04 | 4 | 23 |
| Spleen | 1929 ± 173 | 2 | 34 | 0.04 ± 0.003 | 2 | 34 |
| Liver | 3763 ± 2121 | 48 | ND | 1.7 ± 0.84 | 48 | ND |
| Heart | 1750 ± 107 | 2 | 28 | 0.072 ± 0.015 | 4 | 27 |
| Lungs | 2306 ± 202 | 4 | 32 | 0.09 ± 0.005 | 2 | 47^{‡} |
| Thyroid | 4.6 × 10⁵ ± 1.4 × 10⁵ | 48 | ND | 1.8 ± 0.47 | 48 | ND |

ND = positive slope resulting from accumulation of test article, half-life not calculated.
^{‡}Calculation of tissue half-life required exclusion of an outlier data point; overall trend consistent with elimination rather than accumulation; R²= 0.992.

### Discussion

### Key Points

The elimination half-life (t_{1/2}) of SC Replagal® was approximately 43 hr.
Tissue biodistribution was comparable with previous studies, namely Example 1, and provided an excellent comparison with IV-treated animals in a parallel experiment (Example 3).

### References for Example 2

Alroy J, S Sabnis, and J B Kopp. 2002. Renal pathology in Fabry disease. J Am Soc Nephrol, 13: S134-S138.
Cho M E and J B Kopp. 2004. Fabry disease in the era of enzyme replacement therapy: a renal perspective. Pediatr Nephrol, 19(6): 583-593.
Desnick R J, M Banikazemi, M Wasserstein. 2002. Enzyme replacement therapy for Fabry disease, an inherited nephropathy. Clinical Nephrology, 57(1): 1-8.
Sessa A, M Meroni, G Battini, A Maglio, M Nebuloni, A Tosoni, V Panichi, and B Bertagnolio. 2002. Renal transplantation in patients with Fabry disease. Nephron, 91(2): 348-351.
Tanaka M, T Ohashi, M Kobayashi, Y Eto, N Miyamura, K Nishida, E Araki, K Itoh, K Matsushita, M Hara, K Kuwahra, T Nakano, N Yasumoto, H Nonoguchi, and K Tomia. 2005. Identification of Fabry's disease by the screening of α-galactosidase A activity in male and female patients. Clinical Nephrology, 64(4): 281-287.
Thurberg B L, H Rennke, R B Colvin, S Dikman, R E Gordon, A B Collins, R J Desnick, and M O'Callaghan. 2002. Globotriaosylceramide accumulation in the Fabry kidney is cleared from multiple cell types after enzyme replacement therapy. Kidney International, 62(6): 1933-1946.
Winters J L, A A Pineda, B C McLeod, and K M Grima. 2000. Therapeutic apheresis in renal and metabolic diseases. J Clin Apheresis, 15(1-2): 53-73.

### Example 3

### Pharmacokinetics and Biodistribution of Intravenous [¹²⁵]-Replagal® in Cannulated Rats

This experiment was carried out to examine pharmacokinetic and biodistribution data following adult male jugular-vein cannulated (JVC) Sprague-Dawley rats injected intravenously with 1.0 mg/kg ¹²⁵I-Replagal® (agalsidase alfa) in order to assess serum pharmacokinetics (PK) and tissue biodistribution (BD) over an extended duration (48 hr). Skin (near “injection site” and right thigh), testes, kidneys, spleen, liver, heart, lungs, and thyroid were harvested from each rat. Several serum samples were collected from each animal during the experiment to assess circulating levels of ¹²⁵I-Replagal®. Example 2 describes examination of subcutaneous Replagal® under similar experimental conditions.

### Experimental Design

Data from this experiment documented the bioavailability and tissue biodistribution of the test article at a single dose level (1.0 mg/kg) following intravenous administration. Blood sampling was performed via the externalized venous catheter. Biodistribution of the test article in skin, injection site, testes, kidneys, spleen, liver, heart, lungs, and thyroid was evaluated by gamma counting. Summary pharmacokinetic (PK) and biodistribution data was analyzed and compared with previous results, especially those described in Example 2, a parallel study examining PK and BD following subcutaneous ¹²⁵I-Replagal® under similar experimental conditions.

The experiments described were designed to further evaluate the equivalence of subcutaneous administration by providing parallel PK/BioD data from intravenous injection. Previous results suggested that subcutaneous Replagal® preferentially localized to the kidneys and heart compared to intravenously-dosed animals (Example 1).

### Materials and Methods

### Animals

Jugular vein cannulated (JVC) Sprague-Dawley rats were obtained from Taconic (Germantown, N.Y., USA) at 6-8 weeks of age. As described by the supplier, surgical modification involved the cannulation of the right common jugular vein with 0.023-inch (ID) polyethylene tubing equipped with a silicone rubber intravascular tip and secured with nylon sutures. The remaining 25 mm of cannula was passed beneath the clavicle and externalized between the scapulae using a small midline incision. An anchoring bead was secured along with the skin edges using stainless steel wound clips. A sterile, stainless steel pin sealed the cannulae and was removed for venous access. The average dead volume of the catheter was 30 μL. Animals were housed singly with free access to food and water before and during the experiment. Environmental enrichment was provided via food supplementation and Nylabones®. A total of twenty rats were selected for this experiment; 19 were injected with the iodinated test article as planned. The rat designated B5 (5th animal in Group B) had chewed off his catheter prior to dosing and was sacrificed as a control animal without injection. Tissue and blood were collected as outlined below for experimental animals. These studies complied with USDA regulations and the approved procedures outlined in the institutional Animal Care and Use Protocol (ACUP) 46, entitled “Injections of Therapeutic Proteins in Mice and Rats.”

### Test Article

Pre-filled vials of Replagal® (agalsidase alfa), lot # FG923-004, were obtained from Shire Human Genetic Therapies (Cambridge, Mass.). The concentration was 1.0 mg protein per ml of storage buffer, with a total of 20 mg available for these experiments. The specific enzymatic activity of lot # FG923-004 was reported at 3.2×106 U/mg. Radioiodinated ¹²⁵I-Replagal® was utilized as a tracer for these pharmacokinetic and biodistribution experiments. Iodination using the lactoperoxidase method (Parker, 1990; Marchalonis, 1969) was performed by PerkinElmer (Billerica, Mass., USA) with 500 μg of Replagal®. The final iodinated product contained 25 μCi/mL or approximately 55,200 CPM/μL. Mean dose volume was 0.26 mL per rat. Dosing solutions consisted of unlabeled Replagal® mixed with ¹²⁵I-Replagal® for an approximate radioactivity of 14,500,000 CPM per rat. Both cold and iodinated Replagal® stocks were stored at 4° C. until use.

### Animal Dosing Procedures

Animals were passively restrained in plastic Decapicone® bags (Braintree Scientific, Braintree, Mass.), secured around the tail using 2″-binder clips, for all dosing and sampling. A small triangular opening was made in the plastic bag, through which the catheter was accessed. Baseline blood samples (0.25 mL) were removed via the catheter using a 23-gauge, ½″ aluminum hub blunt needle (Kendall Tyco Healthcare, Mansfield, Mass.) attached to a 1.0-ml plastic disposable syringe (immediately prior to dosing. Intravenous dosing was performed using a clean 23-gauge, ½″ aluminum hub blunt needle (Kendall Tyco Healthcare, Mansfield, Mass.) attached to a 1.0-ml plastic disposable syringe pre-filled with the appropriate dose volume. Intravenous dosing was well tolerated, with no obvious discomfort during or immediately following dose administration.

### Serial Blood Collection

At each time point, the sterile pin sealing the external catheter was gently removed while the rat was safely restrained in a Decapicone®. A 23-gauge blunt needle attached to a 1.0-ml plastic disposable syringe was inserted into the open catheter and gentle pressure employed to remove 0.25 mL of blood. After sufficient sample was obtained, the plastic syringe was removed, leaving the needle in place, and the blood sample transferred to a 0.4-ml serum separator microtainer tube (BD Bioscience, Franklin Lakes, N.J.). A separate syringe contained sterile saline was attached to the needle and the catheter was flushed with approximately 30 μL of the sterile solution to prevent clotting in the catheter. The blunt needle was removed from the catheter while gentle pressure was applied below the tip to prevent backflow of blood. The sterile pin was replaced to seal the catheter. Serial samples were obtained on a pre-determined schedule as described below in Table 7. The mean total volume of blood removed over 48 hr was approximately 10% of total circulating blood as shown below in Table 8. Blood samples were immediately centrifuged at 11,000 rpm (8,500×g) in a fixed-angle rotor for 2 min at room temperature. Serum was transferred to labeled 1.2-ml cryovials (Fisher Scientific, Chicago, Ill.) and stored at −80° C. until analysis.

TABLE 7
Blood Sampling Schedule by Group

| | | Group A | Group B | Group C | Group D |
|---|---|---|---|---|---|
| Time | | 2 hr Sac | 4 hr Sac | 24 hr Sac | 48 hr Sac |
| 0* | | X | X | X | X |
| 15 | min | X | X | | |
| 30 | min | X | X | | |
| 60 | min | X | X | X | X |
| 120 | min | X | X | X | X |
| 180 | min | | X | X | |
| 240 | min | | X | X | X |
| 480 | min | | | X | X |
| 24 | h | | | X | X |
| 36 | h | | | | X |
| 48 | h | | | | X |

*Baseline sample removed prior to dosing
X = Blood sample collected
Sac—Sacrifice

TABLE 8
Calculation of % CBV (Circulating Blood Volumes) Sampled

| | | | | Range | |
|---|---|---|---|---|---|
| | Mean | | SEM | Min | Max |
| Animal weight (kg) | 0.257 | ± | 0.007 | 0.247 | 0.285 |
| Circulating Blood Volume | 16.46 | ± | 0.48 | 15.49 | 18.24 |
| (CBV) (mL) | | | | | |
| Percent CBV Sampled per 24 hr | 10.6 | ± | 0.003 | 9.6 | 11.3 |

### Biodistribution in Target Tissues

In order to assess the biodistribution of ¹²⁵I-Replagal®, several target tissues were sampled, including injection site, thigh skin, testes (pooled), kidneys (pooled), spleen, liver, heart, lungs, and thyroid. Each tissue sample was harvested by blunt dissection and placed immediately in a 5-ml plastic conical tube (Fisher Scientific, Chicago, Ill.) for storage. The thyroid was harvested to assess uptake of radiolabelled iodine from systemic circulation not as a target tissue for Replagal®. The skin samples consisted of approximately 1 cm² portion of skin removed either from the scapular region (referred to as injection site for comparison with data from additional studies) or “distal” skin from the right thigh. Immediately after harvest, the entire organ (or pooled organs in the case of the kidneys and testes) was weighed; these values were recorded as “total organ weight (g).” A portion of the testes and liver were removed for analysis as the entire organ would not fit inside a standard RIA (radioimmunoassay) tube. The remaining organs were counted intact after minimal deconstruction in order to fit the tissues inside RIA tubes. No further processing was performed on the tissue samples and the organs were submitted for gamma counting. Each tube containing tissue was weighed on an analytical balance, tared with an empty RIA tube, after gamma counting. This weight was recorded as “sample weight counted (g).”

### Gamma Counting

The radioactivity of serum and tissue samples from rats injected with ¹²⁵I-Replagal® was quantified using a Wallace WIZARD automatic gamma counter (PerkinElmer, Boston, Mass.). Tissue samples and 100 μL serum aliquots were thawed and transferred to 12×55 mm polycarbonate RIA (radioimmunoassay) tubes (PerkinElmer, Boston, Mass.). A pre-programmed protocol was utilized for the analysis. Briefly, the disintegrations per minute (DPM) of each sample were measured over 60 sec. The DPM was then converted to CPM using an internal efficiency algorithm. An aliquot of the dosing solution (100 μL) from each treatment group was analyzed along with the samples to ensure delivery of adequate and appropriate radioactivity for each animal.

### Data Analysis

Serum CPM/mL values were calculated using sample CPM and the known sample volume (0.1 mL). Total organ CPM was based on the relationship=(Sample CPM/Sample weight in g)*(Organ weight in g).

Percent dose in tissue was calculated by dividing the total organ CPM by the dose (CPM) administered to each rat. Non-compartmental serum pharmacokinetic parameters were calculated using WinNonLin Professional, version 5.0.1 (Pharsight, Mountain View, Calif.). The best-fit lambda z curve was selected based on correlation values of R2=0.90 or greater. WNL calculated several key parameters, including: maximal serum radioactivity (Cₘₐₓ), area under the curve extrapolated to infinity (AUC_{inf}), predicted volume of distribution (Vd_{pred}), predicted total clearance (Cl_{pred}), and mean residence time extrapolated to infinity (MRT_{inf}). Bioavailability was calculated manually using the relationship, F(%)=[(AUCᵢᵥ)/AUC_{sc})]*100. Calculated PK parameters are summarized in Table 9. Serum and tissue radioactivity-time curves were created in GraphPad Prism v.4.0 software (San Diego, Calif.) and Microsoft Excel (v. 2003, Redmond, Wash.). Tissue half-life data was calculated using Prism graphs. Briefly, data was transformed using the relationship Y=ln(Y) and analyzed via non-linear regression. The slope of the best-fit curve was designated ke, the elimination rate constant. Tissue half-life was calculated as t_{1/2}=(ln 2)/(−kₑ). Data from Example 2 was included for calculation of bioavailability.

### Results

### Serum Pharmacokinetics

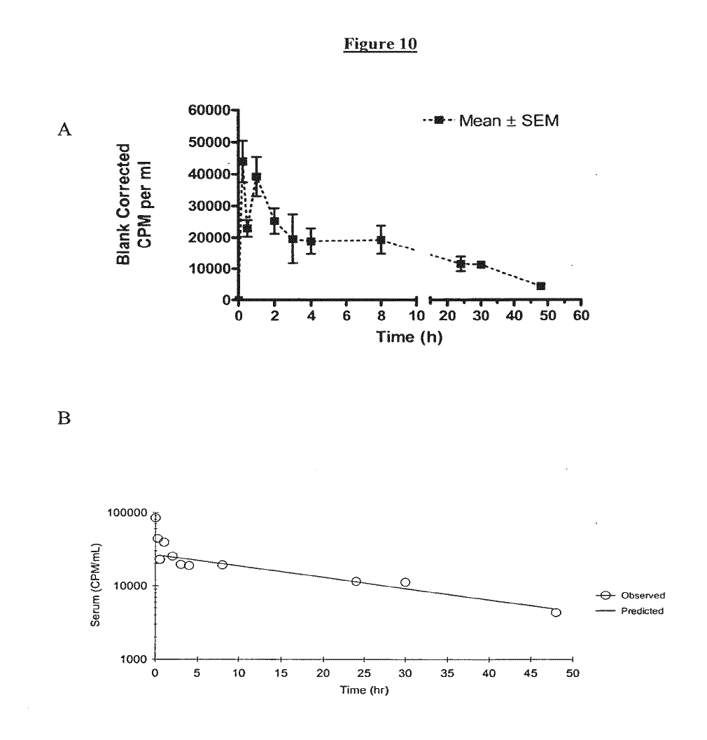

Data for serum radioactivity was analyzed using the WinNonLin (WNL) Professional version non-compartmental functionality (FIG. 10). WNL model #201 (IV Bolus Input) was selected for this data set. A best-fit lambda z line from 0.5 hr to 48 hr was selected with an R2=0.895. Lambda z half-life (t 1/2) was calculated at 19.5 hr.

### Tissue Biodistribution

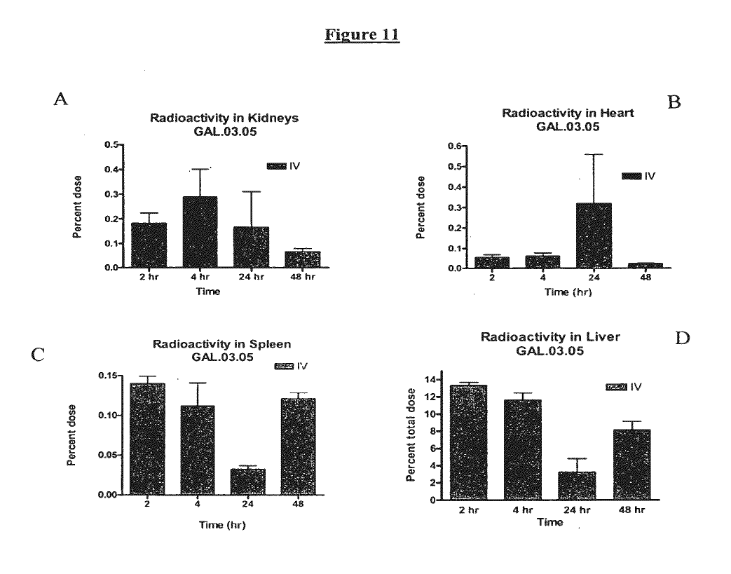

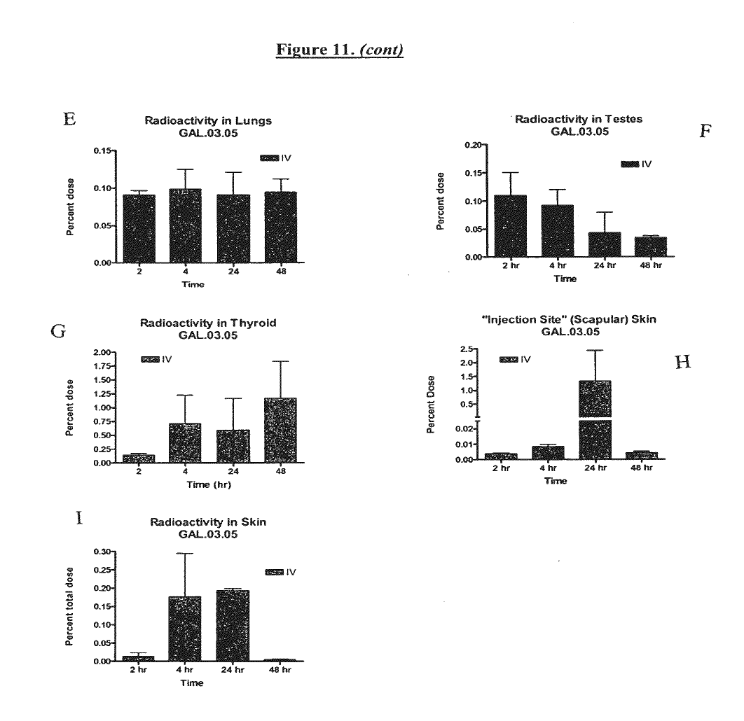

Similar to the serum data presented above, tissue radioactivity was calculated using several methods: CPM per g, total organ CPM, and percent dose. FIG. 11 depicts individual graphs for all harvested tissues (injection site, distal skin, pooled testes, pooled kidneys, spleen, liver, heart, lungs, and thyroid) expressed as percent total dose versus time. Tissue half-life was calculated using the best-fit first order curve matched to log transformed data. Table 10 summarizes biodistribution data gathered in this study. Briefly, radioactivity in the kidneys reaches a maximum at 4 hr (Cₘₐₓ=0.17±0.05% dose) whereas radioactivity in cardiac tissue peaked at 24 hr with Cₘₐₓ=0.56±0.014% dose. Skin samples from the scapular region and the right thigh exhibited accumulation of the test article, as evidenced by the positive slope values calculated from best-fit linear curves. As illustrated in Table 10, most of the tissues demonstrated t½ values less than the duration of the experiment, with the exception of the liver (t_{1/2}=63 hr).

TABLE 9
Summary of Calculated Pharmacokinetic Parameters.

| Parameter | Units | CPM per ml |
|---|---|---|
| Cₘₐₓ | CPM/mL | 43,973 |
| tₘₐₓ | hr | 0.25 |
| kₑ | 1/hr | 0.036 |
| t_{1/2} | hr | 19.5 |
| C₀ | CPM/mL | 84,389 |
| AUC_{inf} | hr*CPM*mL⁻¹ | 764,707 |
| Vd_pred | mL | 588 |
| Cl_{pred} | mL/hr | 20.8 |
| MRT_{inf} | hr | 26 hr |
| F | % | 46 |

Data was calculated using Model 201 in WinNonLin Professional (version 5.0.1) using mean CPM per mL serum data. Values were rounded from raw data for consistency. Bioavailability (F) calculated using the AUC value of 356,763 from Example 2, serum PK after subcutaneous ¹²⁵I-Replagal®.

TABLE 10
Summary of calculated tissue radioactivity
biodistribution parameters.

| | | Percent Dose | | | |
|---|---|---|---|---|---|
| | Cₘₐₓ | | tₘₐₓ | | t½ |
| Tissue | (Total Organ CPM) | Cₘₐₓ | (hr) | ke | (hr) |
| Liver | 1.89 × 10⁶ | 13.4 | 2 | 0.014 | 49.5 |
| Heart | 68,514 | 0.62 | 24 | 0.0095 | 72.9 |
| Thyroid | 201,956 | 0.51 | 4 | 0.02 | 34.7 |
| Skin | 80,889 | 0.11 | 24 | 0.029 | 23.9 |
| Kidneys | 20,360 | 0.16 | 4 | 0.027 | 25.7 |
| Lungs | 2306 | 0.11 | 24 | 0.023 | 30.1 |
| Spleen | 45,943 | 0.23 | 2 | −0.0011 | ND |
| Testes | 3183 | 0.076 | 2 | 0.021 | 33.0 |
| Injection Site | 244,550 | 1.33 | 24 | −0.012 | ND |

Negative kₑ values are a result of a positive slope, indicating accumulation or absence of elimination during the experiment. Therefore, elimination half-life was not determined (ND) for these tissues (injection site and skin).

### Discussion

### Key Points:

Serum pharmacokinetics were similar to those determined in previous studies.
Tissue radioactivity was consistent with previous results, in that liver contained the majority of radioactivity administered. Approximately 20% of the dose was accounted for in this study, although several large compartments were not sampled, for example the GI tract.
Data from this study was analyzed in conjunction with a closely-related experiment, which is described in Example 2 (GAL-02.05 Report #725-1A0-06-727). Based on AUC_{inf} values, the bioavailability of SC ¹²⁵I-Replagal® at 1 mg/kg was calculated at 45%.

### References For Example 3

Cho M E and J B Kopp. 2004. Fabry disease in the era of enzyme replacement therapy: a renal perspective. Pediatr Nephrol, 19(6): 583-593.
Desnick R J, M Banikazemi, M Wasserstein. 2002. Enzyme replacement therapy for Fabry disease, an inherited nephropathy. Clinical Nephrology, 57(1): 1-8.
Sessa A, M Meroni, G Battini, A Maglio, M Nebuloni, A Tosoni, V Panichi, and B Bertagnolio. 2002. Renal transplantation in patients with Fabry disease. Nephron, 91(2): 348-351.
Tanaka M, T Ohashi, M Kobayashi, Y Eto, N Miyamura, K Nishida, E Araki, K Itoh, K Masushita, M Hara, K Kuwahra, T Nakano, N Yasumoto, H Nonoguchi, and K Tomia. 2005. Identification of Fabry's disease by the screening of α-galactosidase A activity in male and female patients. Clinical Nephrology, 64(4): 281-287.
Thurberg B L, H Rennke, R B Colvin, S Dikman, R E Gordon, A B Collins, R J Desnick, and M O'Callaghan. 2002. Globotriaosylceramide accumulation in the Fabry kidney is cleared from multiple cell types after enzyme replacement therapy. Kidney International, 62(6): 1933-1946.
Winters J L, A A Pineda, B C McLeod, and K M Grima. 2000. Therapeutic apheresis in renal and metabolic diseases. J Clin Apheresis, 15(1-2): 53-73.

### Example 4

### Stability of ¹²⁵I-replagal® in Rat Tissues After Injection

An objective of the work described in this example was to characterize the relative stability of ¹²⁵I-Replagal® after subcutaneous (SC) or intravenous (IV) injection in rats via precipitation with Trichloroacetic acid.

### Experimental Design

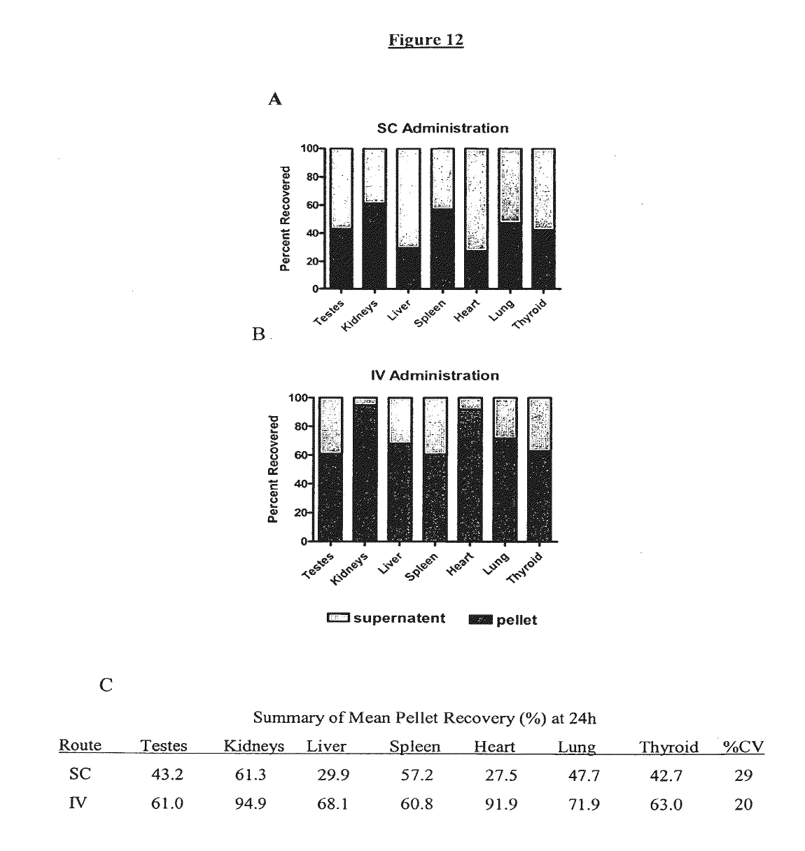

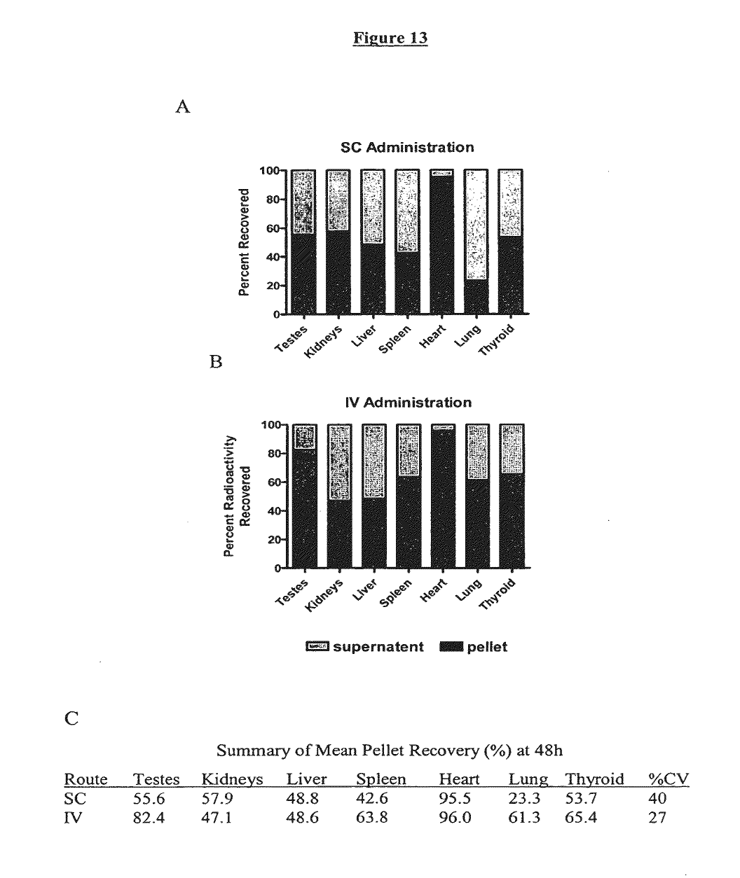

The use of iodinated test articles as tracers in rodent pharmacokinetic (PK) and biodistribution (BD) studies is an accepted and established procedure. However, following injection the stability of the radiolabel is always in question. One approach to quantifying the relative level of intact ¹²⁵I-Replagal® is precipitation of tissue homogenate with trichloroacetic acid (TCA). For this study kidney, heart, liver, spleen, lung, thyroid, and testes were collected from rats 24 h and 48 h after treatment with either SC or IV 1 mg/kg ¹²⁵I-Replagal®. An aliquot of homogenate was mixed with an equal volume of 20% TCA v/v and vortexed. After centrifugation, the pellet and supernatant were analyzed separately for the presence of ¹²⁵I-Replagal® via gamma counting. Results suggest that the majority (>65%) of ¹²⁵I-Replagal® is TCA-precipitable after injection by either SC or IV routes. FIGS. 12 and 13 provide a summary of TCA precipitable radioactivity in rat tissues 24 hr (FIG. 12) or 48 hr (FIG. 13) after SC (top graph) or IV (bottom graph) and also provides a summary of mean pellet recovery at 24 and 48 hours, respectively.

### Materials And Methods

### Animals

Jugular vein cannulated (JVC) Sprague-Dawley rats were obtained from Taconic (Germantown, N.Y., USA) at 6-8 weeks of age. As described by the supplier, surgical modification involved the cannulation of the right common jugular vein with 0.023-inch (ID) polyethylene tubing equipped with a silicone rubber intravascular tip and secured with nylon sutures. The remaining 25 mm of cannula was passed beneath the clavicle and externalized between the scapulae using a small midline incision. An anchoring bead was secured along with the skin edges using stainless steel wound clips. A sterile, stainless steel pin sealed the cannulae and was removed for venous access. The average dead volume of the catheter was 30 μL. Animals were housed singly with free access to food and water before and during the experiment. Environmental enrichment was provided via food supplementation and Nylabones®. A total of twelve rats were obtained for this experiment; all were injected with the iodinated test article as planned (see Table 11). Tissue and blood were collected as outlined below for experimental animals. These studies complied with USDA regulations and the approved procedures outlined in the institutional Animal Care and Use Protocol (ACUP) 46, entitled “Injections of Therapeutic Proteins in Mice and Rats.”

### Research Methods

### Test Article

Pre-filled vials of Replagal® (agalsidase alfa), lot # FG923-004, were obtained from Shire Human Genetic Therapies (Cambridge, Mass.). The concentration was 1.0 mg protein per ml of storage buffer, with a total of 20 mg available for these experiments. The specific enzymatic activity of lot # FG923-004 was reported at 3.2×10⁶ U/mg. Radioiodinated ¹²⁵I-Replagal® was utilized as a tracer for these PK and BD experiments. Iodination using the lactoperoxidase method (Parker, 1990; Marchalonis, 1969) was performed by PerkinElmer (Billerica, Mass., USA) with 500 μg of Replagal®. The final iodinated product contained 25 μCi/mL or approximately 55,200 counts per minute (CPM)/μL. Mean dose volume was 0.31±0.01 mL per rat. Dosing solutions consisted of unlabeled Replagal® mixed with ¹²⁵I-Replagal® for a specific activity of 25,647,428 CPM per mL. The mean dose per rat was 7,867,349±176,738 CPM. Both cold and iodinated Replagal® stocks were stored at 4° C. until use.

### Animal Dosing Procedures

Animals were restrained in plastic Decapicone® bags (Braintree Scientific, Braintree, Mass.), secured around the tail using 2″-binder clips, for all dosing and sampling. A small triangular opening was made in the plastic bag, through which the catheter was accessed. Baseline blood samples (0.25 mL) were removed via the catheter using a 23-gauge, ½″ aluminum hub blunt needle (Kendall Tyco Healthcare, Mansfield, Mass.) attached to a 1.0-ml plastic disposable syringe (immediately prior to dosing. IV dosing was performed using a clean 23-gauge, ½″ aluminum hub blunt needle (Kendall Tyco Healthcare, Mansfield, Mass.) attached to a 1.0-ml plastic disposable syringe pre-filled with the appropriate dose volume. Dosing was well tolerated, with no obvious discomfort during or immediately following administration.

TABLE 11
Summary of Experimental Groups

| Group ID | N | Route | Dose | Test Article |
|---|---|---|---|---|
| A | 6 | SC | 1 mg/kg | ¹²⁵I-Replagal ® |
| B | 6 | IV | 1 mg/kg | ¹²⁵I-Replagal ® |

### Tissue Harvest and Processing

In order to assess the BD of ¹²⁵I-Replagal® several target tissues rats were sacrificed at 24 h (#A1-#A3; #B1-B3) and 48 h (#A4-A6; B4-B6) post-injection (Table 12). The testes, kidneys, spleen, liver, heart, lungs, and thyroid were harvested for analysis. Each tissue sample was harvested by blunt dissection and placed immediately in a 5-ml plastic conical tube (Fisher Scientific, Chicago, Ill.). The thyroid was harvested to assess uptake of radiolabelled iodine from systemic circulation not as a target tissue for Replagal®. Immediately after harvest, the entire organ (or pooled organs in the case of the kidneys and testes) was weighed; these values were recorded as “total organ weight (g).” Tissues were then homogenized in preparation for further analysis.

Precipitation with Trichloroacetic Acid (TCA)

In order to assess the relative stability of the injected ¹²⁵I-Replagal®, tissue homogenate was kept precipitated with trichloroacetic acid (TCA). Briefly, each organ was homogenized in 1.0 mL of reverse-osmosis purified deionized (RO/DI) water using a Fisher PowerGen™ 125 handheld generator equipped with disposable polycarbonate saw-tooth wands for approximately 1 min. Wands were discarded between samples to prevent contamination. A 200 μL aliquot of homogenate was transferred to 0.8 mL microcentrifuge tube and mixed with an equal volume of 20% TCA (v/v) in RO/DI water. The mixture was vortexed on maximum speed for 30 sec and then centrifuged at 7000×g for 2 min to pellet TCA-precipitable material. A 100 μL aliquot of the supernatant was transferred to a clean radioimmunoassay (RIA) tube for gamma counting. Any remaining supernatant was decanted to waste. The pellet was transferred to an RIA tube and resuspended in 100 μL tetraethylammonium hydroxide (TEAH) using plastic spatulas. Samples were loaded immediately into the gamma counter for analysis.

### Gamma Counting

The radioactivity of tissue samples from rats injected with ¹²⁵I-Replagal® was quantified using a Wallace WIZARD automatic gamma counter (PerkinElmer, Boston, Mass.).

Tissue samples were loaded into 12×55 mm polycarbonate RIA (radioimmunoassay) tubes (PerkinElmer, Boston, Mass.). A pre-programmed protocol was utilized for the analysis. Briefly, the disintegrations per minute (DPM) of each sample were measured over 60 sec. The DPM was then converted to CPM using an internal efficiency algorithm.

### Data Analysis

The relative radioactivity in the precipitable (pellet) and non-precipitable protein (supernatant) was calculated as percent of the combined CPM. Values from like tissues were combined to provide a mean±SEM percent. Relative radioactivity in pellet vs. supernatant between the treatment groups was calculated using MS Excel for Windows XP Professional (Redmond, Wash.). Graphs were created using Prism v.4 software (GraphPad, San Diego, Calif.).

### Discussion

Recovery of radioactivity using TCA precipitation suggests a relatively intact test article within 48 h after single injection.

Significant variability was observed between tissues and between subjects. CV % for this experiment ranged between 19% and 55% for the eight data analysis groups (n=3 rats per group).

### Example 5

Investigation of Pharmacokinetics (pk) and Biodistribution (bd) of Subcutaneous (sc) Versus Intravenous (iv) ¹²⁵I-replagal® In Fabry Mice.

### Experimental Design

Data from this experiment describe the bioavailability and tissue BD of the Replagal® (Shire Human Genetic Therapies, Cambridge, Mass.) at a single dose level (1.0 mg/kg) following subcutaneous (SC) or intravenous (IV) administration in Fabry mice. Blood sampling was performed via intracardiac puncture at sacrifice. BD of the test article in skin, injection site, testes, kidneys, spleen, liver, heart, lungs, and thyroid was evaluated by gamma counting.

These experiments were designed to further evaluate the equivalence of SC administration by providing parallel PK/BD data from intravenous injection in a mouse model of Fabry disease.

### Material and Methods

### Animals

Gla^{tmlkul}/J mice, referred to herein as Fabry mice, were bred in-house from breeding pairs obtained from Jackson Laboratories, Bar Harbor, Me. All mice were genotyped via PCR on DNA extracted from tail or ear samples shortly after weaning. Briefly, tissue samples from tail snips or ear punches collected from conventional ear marking were digested using the DNeasy Tissue Kit (Qiagen, Valencia, Calif.; lot #4096816). DNA was amplified using three primers that target sequences flanking exon 3 of the murine Gla gene; this portion of the Gla gene is deleted in affected males and carrier females, thereby producing a different length amplicon for these two genotypes. PCR products were separated using bufferless, pre-cast 4% agarose E-gels pre-stained with ethidium bromide (Invitrogen) for 30 minutes at 60 volts. A UV gel documentation system was utilized to visualize DNA fragments separated on the gel. After genotyping, mice were assigned colony numbers and tracked using the BigBench™ colony software (Vancouver, B.C, Canada). Tissue and blood were collected as outlined below for experimental animals. These studies complied with USDA regulations and the approved procedures outlined in the institutional Animal Care and Use Protocol (ACUP) 46, entitled “Injections of Therapeutic Proteins in Mice and Rats.”

### Test Article

Replagal® (agalsidase alfa) drug substance at 51 mg/mL, lot #NB4249-48, was obtained from Shire Human Genetic Therapies (Cambridge, Mass.). Radioiodinated test article (¹²⁵I-Replagal®) was utilized as a tracer for these PK and BD experiments. Iodination using the lactoperoxidase method (Parker, 1990; Marchalonis, 1969) was performed by PerkinElmer (Billerica, Mass., USA) with 500 μg of Replagal® drug substance. The final iodinated product contained 100 μCi/mL or approximately 1.28 μCi per μg of protein. Dosing solutions consisted of unlabeled Replagal® mixed with ¹²⁵I-Replagal® for an approximate radioactivity of 13,000,000 CPM per mL. Mean dose volume was 0.11 mL per mouse, for an approximate dose of 1,500,000 CPM per animal. Iodinated Replagal® stocks were stored at 4° C., whereas cold 30 mg/mL Replagal® was maintained at −80° C. until use.

### Animal Dosing Procedures

Mice were placed in plastic restrainers for intravenous dosing. Subcutaneous dosing was performed using manual restraint the mice. Both routes of dosing were well tolerated, with no obvious discomfort during or immediately following dose administration. See Table 12 for summary of experimental groups.

TABLE 12
Summary of Experimental Groups

| Group | Dose (mg/kg) | Route | Test Article | Lot # | N |
|---|---|---|---|---|---|
| A | 1.0 | IV | Replagal ® | FG923-004 | 18 |
| B | 1.0 | IV | Vehicle | n/a | 3 |
| C | 1.0 | SC | Replagal ® | FG923-004 | 18 |
| D | 1.0 | SC | Vehicle | n/a | 3 |

### Blood Collection

Mice were sacrificed using carbon dioxide euthanasia. Approximately 0.5 ml of whole blood was obtained via cardiac puncture using a 25 g ⅝-inch needle attached a 1.0-ml syringe. Blood samples clotted at room temperature for at least 10 min and were centrifuged at 11,000 rpm (8,500×g) in a fixed-angle rotor for 2 min at room temperature to collect serum. An aliquot of each serum sample was transferred to labeled 1.2-ml cryovials (Fisher Scientific, Chicago, Ill.) and analyzed via gamma counting.

### Biodistribution in Target Tissues

In order to assess the BD of ¹²⁵I-Replagal® several target tissues were sampled including injection site, kidneys, spleen, liver, heart, and thyroid. Each tissue sample was harvested by blunt dissection and placed immediately in a 5-ml plastic conical tube (Fisher Scientific, Chicago, Ill.) for storage. The thyroid was harvested to assess uptake of radiolabelled iodine from systemic circulation not as a target tissue for Replagal® disposition. The injection site samples consisted of approximately 1 cm² portion of skin removed from the scapular region containing the site of injection. Immediately after harvest, the entire organ (or pooled organs in the case of the kidneys) was weighed; these values were recorded as “total organ weight (g).” A portion of the liver was removed for analysis since the entire intact organ would not fit inside a standard RIA (radioimmunoassay) tube. The weight of this piece was recorded separately as “sample weight (g)”. The remaining organs were counted intact after minimal deconstruction in order to fit the tissues inside RIA tubes. Control tissues were harvested from an untreated mouse. Representative liver and kidney samples were homogenized and a 200 μL aliquot precipitated with equal volumes of 20% trichloroacetic acid (TCA). The precipitate was centrifuged to collect insoluble proteins into a pellet. The supernatant and pellet were transferred to separate RIA tubes for analysis. A 100 μL aliquot of homogenate was counted to calculate percent recovery of radioactivity in pellet and supernatant.

### Gamma Counting

The radioactivity of serum and tissue samples from mice injected with ¹²⁵I-Replagal® was quantified using a Wallace WIZARD automatic gamma counter (PerkinElmer, Boston, Mass.). Tissue samples and 100 μL serum aliquots were thawed, if necessary, and transferred to 12×55 mm polycarbonate RIA (radioimmunoassay) tubes (PerkinElmer, Boston, Mass.). A pre-programmed protocol was utilized for the analysis. Briefly, the disintegrations per minute (DPM) of each sample were measured over 60 sec. The DPM was then converted to CPM using an internal efficiency algorithm. An aliquot of the dosing solution (100 μL) from each treatment group was analyzed along with the samples to calculate dose delivered to each mouse.

### Data Analysis

Serum CPM/mL Values Were Calculated Sample CPM and the known Sample Volume (0.1 mL).

Total organ CPM=(Sample CPM/Sample weight in g)*(Organ weight in g). Percent dose in tissue=(total organ CPM)/(dose CPM)*100

Non-compartmental serum PK parameters were calculated using WinNonLin Professional, version 5.0.1 (Pharsight, Mountain View, Calif.). The best-fit lambda z curve was selected based on correlation values of R²=0.90 or greater. WNL calculated several key parameters, including: maximal serum radioactivity (Cₘₐₓ), area under the curve extrapolated to infinity (AUC_{inf}), predicted volume of distribution (Vd_{pred}), predicted total clearance (Cl_{pred}), and mean residence time extrapolated to infinity (MRT_{inf}). Fraction available in serum was calculated manually using the relationship:

F(%)=[AUCᵢᵥ)/AUC_{sc})]*100.

TCA-precipitated tissue data was expressed as percent recovered in pellet (% pellet) and supernatant (% supernatant). Serum and tissue radioactivity-time curves were created in GraphPad Prism v.4.0 software (San Diego, Calif.) and Microsoft Excel (v. 2003, Redmond, Wash).

### Discussion

### Key Points:

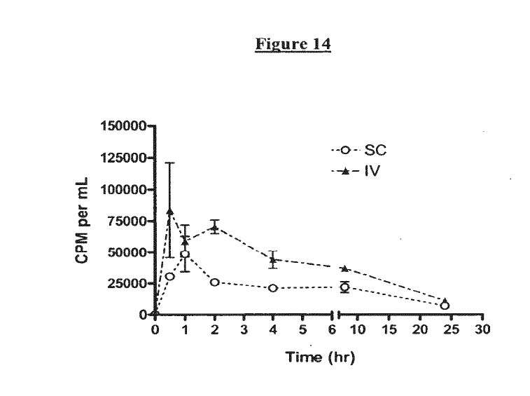

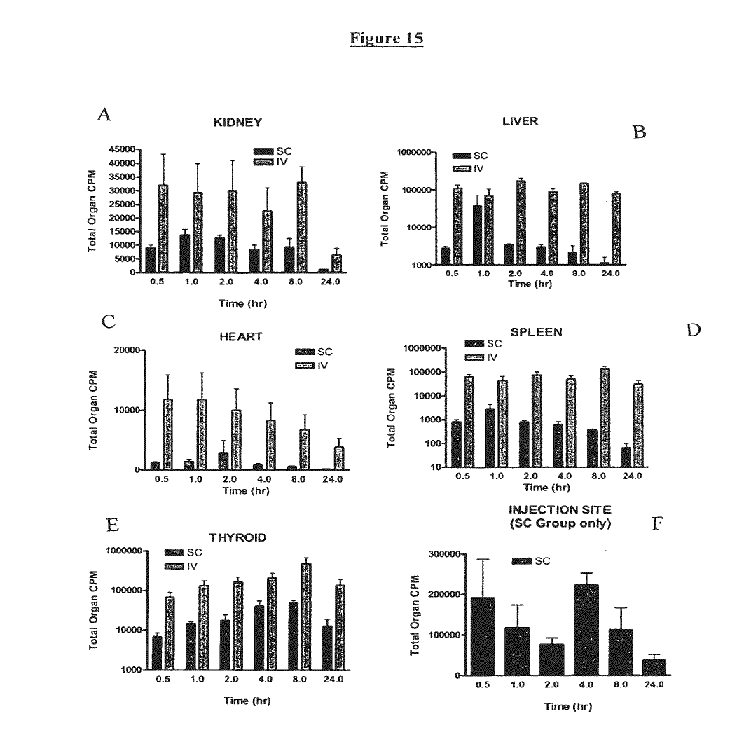

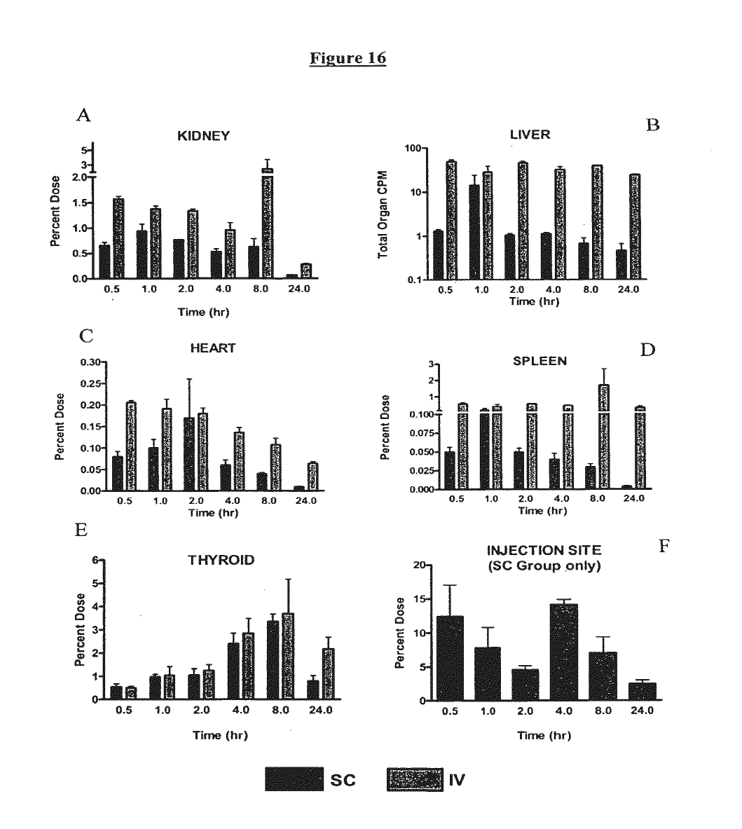

Serum PK appeared similar to findings from previous cannulated rat PK/BD studies (see FIG. 14).
Tissue BD was also very similar to data previously attained on rat studies, see FIG. 15, which demonstrates:
   SC injection achieved comparable levels in kidney versus bolus IV injection.
   Liver radioactivity was greatly reduced in SC-treated mice compared to IV injection, with the exception of the lh time point.
   Thyroid radioactivity was low (<5% of dose) and the overall pattern of accumulation was very similar between SC and IV-treated mice.
TCA-precipitable radioactivity was similar to previous data, with an overall pellet recovery of approximately 42% and 75% for SC and IV-treated mice, respectively, see FIGS. 16 and 17.
Approximately 20% of the dose was accounted for in this study, although several large compartments were not sampled, including the GI tract.

### Example 6

### Steady-State PK of ¹²⁵I-Replagal® in Rats

### Major Objectives

Achieve steady-state serum levels of ¹²⁵I-Replagal® in rats.
Characterize tissue radioactivity after multiple injections of ¹²⁵I-Repalagal™.

### Experimental Design

Jugular-vein cannulated (JVC) rats were injected either SC or IV with ¹²⁵I-Replagal® to investigate the required dose and frequency to achieve steady-state serum pharmacokinetics (PK). Tables 13 and 14 show the experimental design. The study lasted for one week and serum was collected several times per day to assess pharmacokinetics of the test article. All samples were analyzed for the presence of ¹²⁵I-Replagal® using a gamma counter. These studies, complement previous studies, by providing a more comprehensive, long-term view of Replagal® disposition following multiple SC or IV injections.

TABLE 13
Experimental Design for Study.

| | Dose Per | | Injections | | | |
|---|---|---|---|---|---|---|
| Group | Injection | Route | per week | Test Article | Lot # | Rat ID |
| A | 1.0 mg/kg | SC | Two | ¹²⁵I- | PAD- | #A1-A9 |
| B | 1.0 mg/kg | IV | Two | Replagal ® | 4344-27 | #B1-B9 |
| C | 1.0 mg/kg | SC | Four | | | #C1-C9 |
| D | 1.0 mg/kg | IV | Four | | | #D1-D9 |

TABLE 14
Experimental Design for Study.

| | Dose Per | | Injections | | PAD | |
|---|---|---|---|---|---|---|
| Group | Injection | Route | per week | Test Article | Lot # | ID |
| A | 0.5 mg/kg | SC | Two | ¹²⁵I- | PAD- | #A1-A9 |
| B | 0.5 mg/kg | IV | Two | Replagal ® | 4344- | #B1-B9 |
| C | 0.25 mg/kg | SC | Four | | 27 | #C1-C9 |
| D | 0.25 mg/kg | IV | Four | | | #D1-D9 |

### Material and Methods

### Animals

Jugular vein cannulated (JVC) Sprague-Dawley rats were obtained from Taconic (Germantown, N.Y., USA) at 6-8 weeks of age. As described by the supplier, surgical modification involved the cannulation of the right common jugular vein with 0.023-inch (ID) polyethylene tubing equipped with a silicone rubber intravascular tip and secured with nylon sutures. The remaining 25 mm of cannulae was passed beneath the clavicle and externalized between the scapulae using a small midline incision. An anchoring bead was secured along with the skin edges using stainless steel wound clips. A sterile, stainless steel pin sealed the cannulae and was removed for venous access. The average dead volume of the catheter was 30 μL. Animals were housed singly with free access to food and water before and during the experiment. Environmental enrichment was provided via food supplementation and Nylabones®. A total of twenty-five were purchased for this experiment; 21 were injected with the iodinated test article as planned. No morbidity or mortality of animals occurred during this study. Untreated animals were sacrificed as controls or used to train lab personnel on proper injection techniques. Tissue and blood were collected as outlined below for experimental animals. These studies complied with USDA regulations and the approved procedures outlined in the institutional Animal Care and Use Protocol (ACUP) 46, entitled “Injections of Therapeutic Proteins in Mice and Rats.”

### Test Article

Replagal® was obtained from Shire Human Genetic Therapies (Cambridge, Mass.) at a concentration of 51 mg/mL, lot# PAD4344-17, source material lot# (DS) 302-010. Radioiodinated ¹²⁵I-Replagal® was utilized as a tracer for these pharmacokinetic and biodistribution experiments. Iodination using the lactoperoxidase method (Parker, 1990; Marchalonis, 1969) was performed by PerkinElmer (Billerica, Mass., USA) with 500 μg of Replagal®. The final iodinated product contained 25 μCi/mL or approximately 55,200 CPM/μL. Mean dose volume was 0.26 mL per rat. Dosing solutions consisted of unlabeled Replagal® mixed with ¹²⁵I-Replagal® for an approximate radioactivity of 14,500,000 CPM per rat. Both cold and iodinated Replagal® stocks were stored at 4° C. until use.

### Animal Dosing Procedures

Animals were restrained in plastic Decapicone® bags (Braintree Scientific, Braintree, Mass.), secured around the tail using 2″-binder clips, for all dosing and sampling. A small triangular opening was made in the plastic bag, through which the catheter was accessed. Baseline blood samples (0.25 mL) were removed via the catheter using a 23-gauge, ½″ aluminum hub blunt needle (Kendall Tyco Healthcare, Mansfield, Mass.) attached to a 1.0-ml plastic disposable syringe (immediately prior to dosing. Intravenous dosing was performed using a clean 23-gauge, ½″ aluminum hub blunt needle attached to a 1.0-ml plastic disposable syringe pre-filled with the appropriate dose volume. Subcutaneous dosing was performed using a 1.0-ml plastic syringe attached to a ⅝″-28 g needle pre-filled with the appropriate dose volume. Intravenous and subcutaneous dosing was well tolerated, with no obvious discomfort during or immediately following dose administration.

### Serum Pharmacokinetics

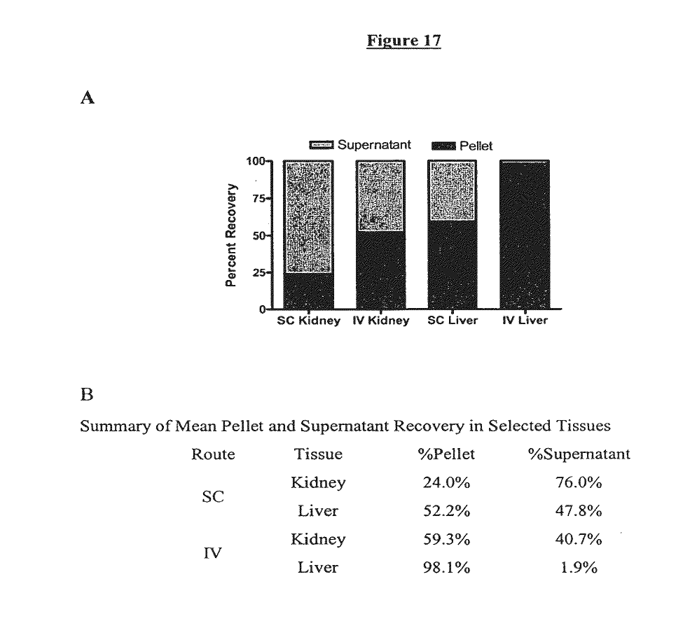

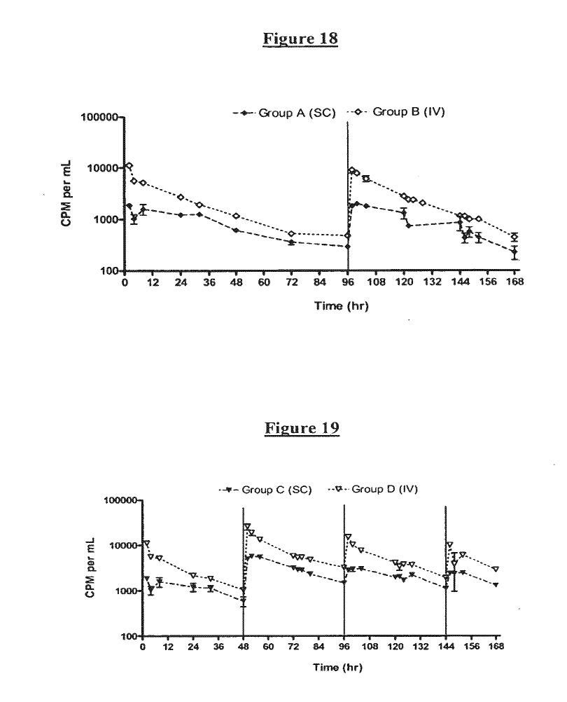

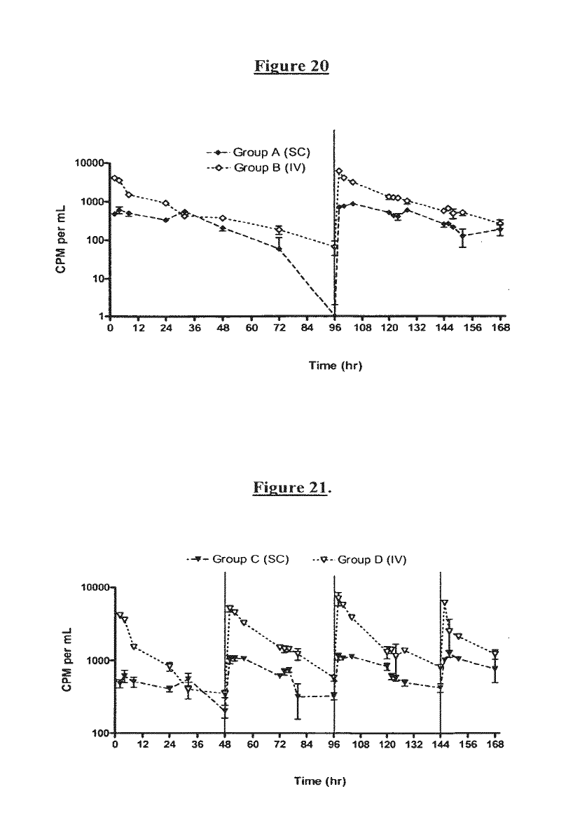

At the specified time point, a 0.25 mL blood samples was withdrawn from the rats via the jugular catheter and collected in a 0.4 mL Microtainer™ serum separator tube (BD Biosciences). Following sample collection the catheter was flushed with an equal volume of sterile saline to prevent coagulation and associated morbidity. Each blood sample was allowed to clot at room temperature for a maximum of 10 min. The coagulated blood samples were centrifuged at 14,000 rpm (approximately 5000×g) for two minutes at room temperature. A 100 μL aliquot of serum was transferred to an RIA tube for gamma counting. Any remaining serum was held at 4° C. until all the samples had been successfully analyzed. FIGS. 18 and 19 provide results from study group depicted in Table 13. FIGS. 20 and 21 provide results from study group depicted in Table 14.

### Biodistribution in Target Tissues

In order to assess the biodistribution of ¹²⁵I-Replagal® of the study duration groups of n=3 rats were sacrificed from either group every 24 hr following treatment. The liver, kidney, heart, spleen, injection site, and thyroid were harvested for analysis. Each tissue sample was harvested by blunt dissection and placed immediately in a 5-ml plastic conical tube (Fisher Scientific, Chicago, Ill.). The thyroid was harvested to assess uptake of radiolabelled iodine from systemic circulation not as a target tissue for Replagal®. Immediately after harvest, each intact organ was weighed; these values were recorded as “organ weight (g).”

### Gamma Counting

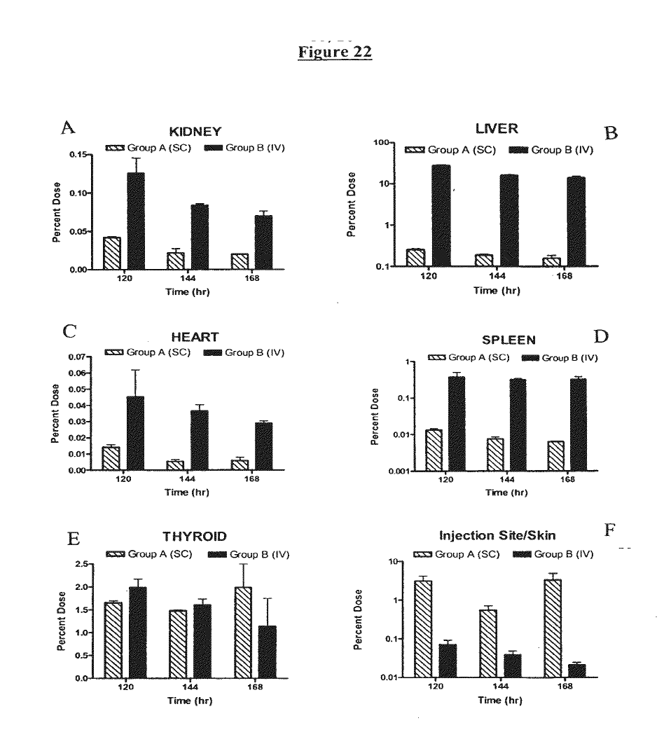

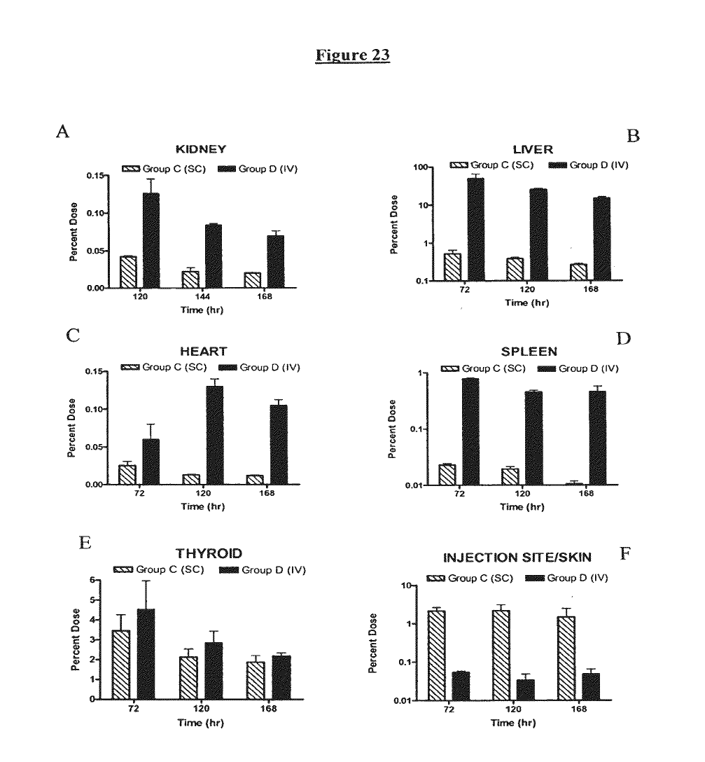

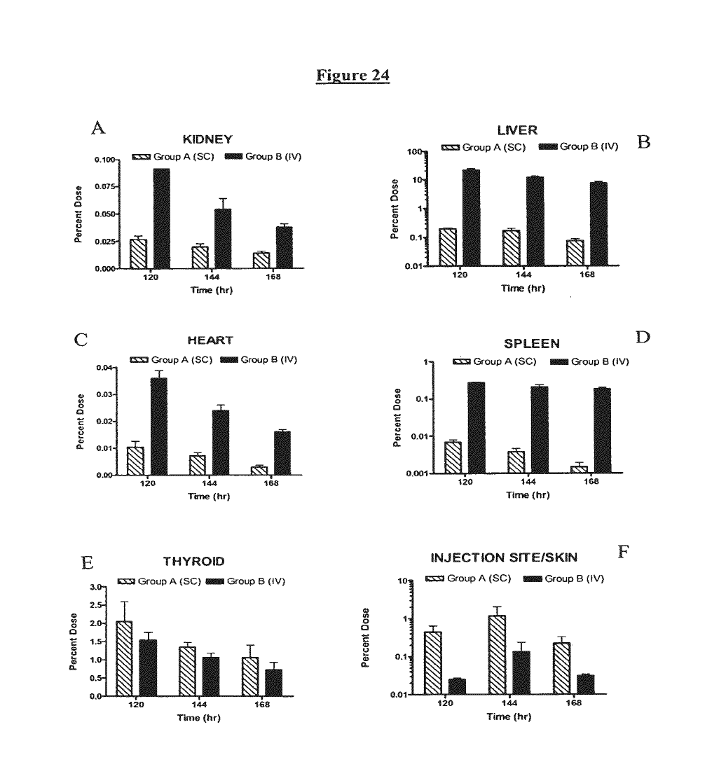

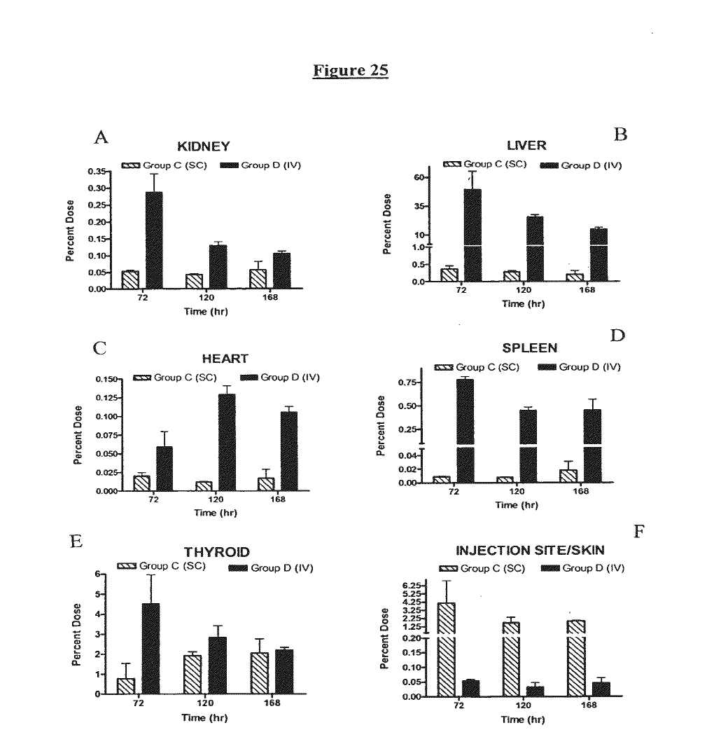

The radioactivity of tissue samples from rats injected with ¹²⁵I-Replagal® was quantified using a Wallace WIZARD automatic gamma counter (PerkinElmer, Boston, Mass.). Tissue samples were loaded into 12×55 mm polycarbonate RIA (radioimmunoassay) tubes (PerkinElmer, Boston, Mass.). A pre-programmed protocol was utilized for the analysis. Briefly, the disintegrations per minute (DPM) of each sample were measured over 60 sec. The DPM was then converted to CPM using an internal efficiency algorithm. Tissue radioactivity data for the study depicted in Table 13 is shown in FIGS. 22 and 23. Tissue radioactivity data for the study depicted in Table 14 is shown in FIGS. 24 and 25.

### Data Analysis

Serum CPM/mL values were calculated sample CPM and the known sample volume (0.1 mL). Total organ CPM was based on the relationship=(Sample CPM/Sample weight in g)*(Organ weight in g). Percent dose in tissue was calculated by dividing the total organ CPM by the dose (CPM) administered to each mouse. Non-compartmental serum pharmacokinetic parameters were calculated using WinNonLin Professional, version 5.0.1 (Pharsight, Mountain View, Calif.) (Table 16). The best-fit lambda z curve was selected based on correlation values of R²=0.90 or greater. WNL calculated several key parameters, including: maximal serum radioactivity (Cₘₐₓ), area under the curve extrapolated to infinity (AUC_{inf}), predicted volume of distribution (Vd_{pred}), predicted total clearance (Cl_{pred}), and mean residence time extrapolated to infinity (MRT_{inf}). Fraction available in serum was calculated manually using the relationship, F(%)=[(AUCᵢᵥ)/AUC_{sc})]*100. Tissue half-life was calculated from the best-fit lambda z line against log—transformed total organ CPM vs. time curves using the relationship t½=(ln 2)/(-slope). Serum and tissue radioactivity-time curves were created in GraphPad Prism v.4.0 software (San Diego, Calif.) and Microsoft Excel (v. 2003, Redmond, Wash.). Table 15 shows calculated parameters from WinNonLin non compartmental analysis of serum PK and Table 16 shows a comparison of tissue serum ratios for selected organs.

TABLE 15
Calculated parameters from WinNonLin noncompartmental analysis of serum PK

| Study ID | | GAL.05.06 | | | | GAL.06.06 | | | |
|---|---|---|---|---|---|---|---|---|---|
| Weekly Dosing Regimen | | 2 × 1 mg/kg* | | 4 × 1 mg/kg^{‡} | | 2 × 0.5 mg/kg* | | 4 × 0.25 mg/kg^{‡} | |
| Parameter | Units | SC | IV | SC | IV | SC | IV | SC | IV |
| Cₘₐₓ | CPM/mL | 2,023 | 9,181 | 2,464 | 10,201 | 885 | 6,273 | 1,245 | 6,154 |
| tₘₐₓ | hr | 4 | 2 | 8 | 2 | 8 | 2 | 4 | 2 |
| λ_{z} t_{1/2} | hr | 24 | 17 | 22 | 13 | 27 | 17 | 39 | 12 |
| AUCₗₐₛₜ | (CPM/mL) * (hr) | 149,957 | 369,692 | 368,721 | 900,402 | 56,118 | 161,392 | 109,971 | 302,364 |
| Cl_{obs} | ml/hr/kg | 12 | 9 | n/a | n/a | 31 | 12 | n/a | n/a |
| Clₛₛ | ml/hr/kg | n/a | n/a | 40 | 18 | n/a | n/a | 9 | 5 |
| Vz_{obs} | ml | 407 | 215 | 1,345 | 433 | 1,141 | 279 | 521 | 92 |
| AUMCₗₐₛₜ | [(hr) * | 11,123,107 | 3,801,844 | n/a | n/a | 809,097 | 1,981,526 | n/a | n/a |
| | (CPM/mL)] * | | | | | | | | |
| | hr | | | | | | | | |
| MRTₗₐₛₜ | hr | 75 | 20 | 32 | 24 | 25 | 19 | 56 | 19 |
| F | % | 41 | | 41 | | 35 | | 36 | |

*Injections at 0 and 96 hrs.
^{‡}Every other day (EOD) dosing.
Cₘₐₓ = maximum observed serum concentration after final injection.
Tₘₐₓ = time Cₘₐₓ was achieved after final injection.
λ_{z} t_{1/2} = half-life in terminal phase (lambda z or λ_{z}).
AUCₗₐₛₜ = area under the CPM/mL vs. time curve without extrapolation to infinity (observed data only)
Cl_{obs} = total serum clearance based on observed data.
Clₛₛ = clearance during steady-state dosing.
Vz_{obs} = volume of distribution in the lambda z (elimination) phase based on observed data
AUMCₗₐₛₜ = area under the first moment vs. time curve based on observed data.
MRTₗₐₛₜ = mean residence time, based on (AUC/AUMC) relationship.
F = relative bioavailability, based on the (AUC_{SC}/AUV_{IV}) relationship.
n/a = not applicable.

TABLE 16
Comparison of tissue serum ratios for selected organs.

| | | | | | | Tissue | |
|---|---|---|---|---|---|---|---|
| | Total Weekly | | | | | to Serum | |
| Dosing | Dose | Tissue AUC [symbol] | | Serum AUC§ | | Ratio† | |
| Regimen | (mg/kg/wk) | SC | IV | SC | IV | SC | IV |

Rat Kidney

| | | | | | | | | |
|---|---|---|---|---|---|---|---|---|
| 4 × 0.25 | mg/kg* | 1 | 86,746 | 164.668 | 109,971 | 302,364 | 0.79 | 0.54 |
| 2 × 0.5 | mg/kg | 1 | 34,707 | 96,514 | 56,118 | 161,392 | 0.62 | 0.60 |
| 2 × 1 | mg/kg | 2 | 95,565 | 201,275 | 149,957 | 369,692 | 0.64 | 0.54 |
| 4 × 1 | mg/kg* | 4 | 233,249 | 546,386 | 368,721 | 900,402 | 0.63 | 0.61 |

Rat Heart

| | | | | | | | | |
|---|---|---|---|---|---|---|---|---|
| 4 × 0.25 | mg/kg* | 1 | 27,195 | 67,097 | 109,971 | 302,364 | 0.25 | 0.22 |
| 2 × 0.5 | mg/kg | 1 | 12,815 | 39,568 | 56,118 | 161,392 | 0.23 | 0.25 |
| 2 × 1 | mg/kg | 2 | 40,752 | 74,433 | 149,957 | 369,692 | 0.27 | 0.20 |
| 4 × 1 | mg/kg* | 4 | 56,757 | 182,381 | 368,721 | 900,402 | 0.15 | 0.20 |

Rat Liver

| | | | | | | | | |
|---|---|---|---|---|---|---|---|---|
| 4 × 0.25 | mg/kg* | 1 | 488,435 | 4.14 × 10⁷ | 109,971 | 302,364 | 4.44 | 136.92 |
| 2 × 0.5 | mg/kg | 1 | 262,164 | 2.17 × 10⁷ | 56,118 | 161,392 | 4.67 | 134.46 |
| 2 × 1 | mg/kg | 2 | 557,129 | 4.3 × 10⁷ | 149,957 | 369,692 | 3.72 | 116.31 |
| 4 × 1 | mg/kg* | 4 | 1.4 × 10⁶ | 9.1 × 10⁷ | 368,721 | 900,402 | 3.80 | 101.07 |

[symbol]

Tissue ACU values are based on total organ CPM vs. time curves.
§Serum AUC values are based on CPM per mL vs. time curves.
†Tissue to serum ratio = (tissue AUC)/(serum AUC).
*This dosing regimen achieved steady-state.

### Discussion

### Serum Pharmacokinetics

Every other day (EOD or 4×/wk) dosing of ¹²⁵I-Replagal® achieved steady-state kinetics with accumulation observed after the third injection.

Mean fraction available (F%) for SC versus IV administration was approximately 38%, with a range of 36 to 41% depending on dose and regimen.

### Tissue Biodistribution

Every other day (EOD or 4×/wk) dosing of ¹²⁵I-Replagal® improved tissue partitioning of SC Replagal®, especially in the kidney.

Overall it appears that splitting a 1 mg/kg/wk dose into smaller more frequent SC injections may provide, at minimum, similar test article levels in organs of interest, especially the kidney, compared to a single weekly injection.

### Example 7

### Major Objectives

To characterize the serum pharmacokinetics and tissue biodistribution of ¹²⁵I-Replagal® over 7 days after a single subcutaneous (SC) or intravenous (IV) injection in rats.

### Experimental Design

### Overview

Jugular-vein cannulated (JVC) rats were injected either SC or IV with 1 mg/kg ¹²⁵I-Replagal® on Day 0. Groups of 6 rats (3 per route) were sacrificed every 24 hr to harvest tissue until Day 7 (168 hr). Serum was collected several times per day to assess pharmacokinetics of the test article. All samples were analyzed for the presence of ¹²⁵I-Replagal® using a gamma counter. This study, complements previous studies, especially studies such as Example 2, by providing a more comprehensive, long-term view of test article disposition following a single SC or IV injection. In previous experiments, the longest duration was 48 hr post-injection; the terminal half-life of SC ¹²⁵I-Replagal® was calculated at 53 hr, suggesting that a multi-day study was required to completely describe elimination. Moreover, the study described in this example study provides a 7 day time course of test article accumulation in key organs including the kidneys, heart, liver, and spleen.

### Materials and Methods

### Animals

Jugular vein cannulated (JVC) Sprague-Dawley rats were obtained from Taconic (Germantown, N.Y., USA) at 6-8 weeks of age. As described by the supplier, surgical modification involved the cannulation of the right common jugular vein with 0.023-inch (ID) polyethylene tubing equipped with a silicone rubber intravascular tip and secured with nylon sutures. The remaining 25 mm of cannulae was passed beneath the clavicle and externalized between the scapulae using a small midline incision. An anchoring bead was secured along with the skin edges using stainless steel wound clips. A sterile, stainless steel pin sealed the cannulae and was removed for venous access. The average dead volume of the catheter was 30 μL. Animals were housed singly with free access to food and water before and during the experiment. Environmental enrichment was provided via food supplementation and Nylabones®. A total of 43 were purchased for this experiment; 42 were injected with the iodinated test article as planned. No morbidity or mortality of animals occurred during this study. Tissue and blood were collected as outlined below for experimental animals. These studies complied with USDA regulations and the approved procedures outlined in the institutional Animal Care and Use Protocol (ACUP) 46, entitled “Injections of Therapeutic Proteins in Mice and Rats.”

### Test Article

Replagal® was obtained from internal sources (PAD) at a concentration of 51 mg/mL, lot# PAD4344-17, source material lot# (DS) 302-010. Radioiodinated ¹²⁵I-Replagal® was utilized as a tracer for these pharmacokinetic and biodistribution experiments. Iodination using the lactoperoxidase method (Parker, 1990; Marchalonis, 1969) was performed by PerkinElmer (Billerica, Mass., USA) with 500 μg of Replagal®. The final iodinated product contained 25 μCi/mL or approximately 55,200 CPM/μL. Mean dose volume was 0.26 mL per rat. Dosing solutions consisted of unlabeled Replagal® mixed with ^{125I}-Replagal® for an approximate radioactivity of 14,500,000 CPM per rat. Both cold and iodinated Replagal® stocks were stored at 4° C. until use.

### Animal Dosing Procedures

Animals were restrained in plastic Decapicone® bags (Braintree Scientific, Braintree, Mass.), secured around the tail using 2″-binder clips, for all dosing and sampling. A small triangular opening was made in the plastic bag, through which the catheter was accessed. Baseline blood samples (0.25 mL) were removed via the catheter using a 23-gauge, ½″ aluminum hub blunt needle (Kendall Tyco Healthcare, Mansfield, Mass.) attached to a 1.0-ml plastic disposable syringe (immediately prior to dosing. Intravenous dosing was performed using a clean 23-gauge, ½″ aluminum hub blunt needle (Kendall Tyco Healthcare, Mansfield, Mass.) attached to a 1.0-ml plastic disposable syringe pre-filled with the appropriate dose volume. Intravenous and subcutaneous dosing was well tolerated, with no obvious discomfort during or immediately following dose administration. A summary of the experimental groups is shown in Table 17.

TABLE 17
Summary of Experimental Groups

| | Dose | | | | | |
|---|---|---|---|---|---|---|
| Group | (mg/kg) | Route | Test Article | Lot # | ID | N |
| A | 1.0 | SC | ¹²⁵I- | PAD-4344-17 | #A1-A21 | 21 |
| | | | Replagal ® | | | |
| B | 1.0 | IV | ¹²⁵I | PAD-4344-17 | #B1-B21 | 21 |
| | | | Replagal ® | | | |

### Serum Pharmacokinetics

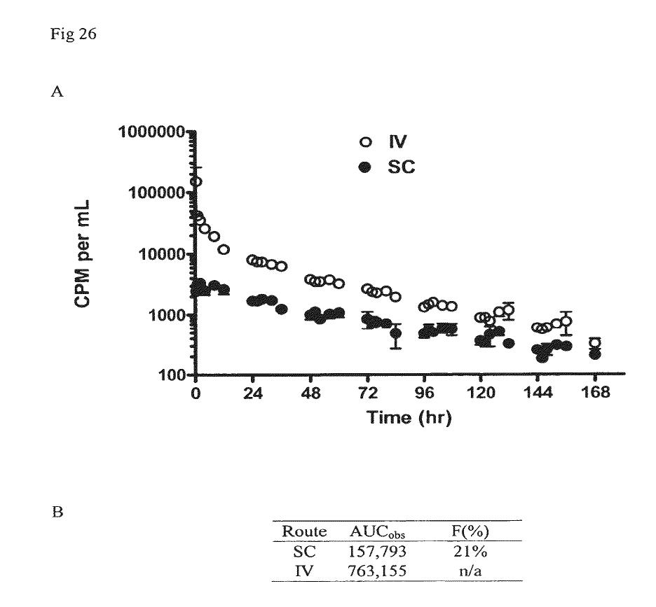

At the specified time point, a 0.25 mL blood samples was withdrawn from the rats via the jugular catheter and collected in a 0.4 mL Microtainer™ serum separator tube (BD Biosciences). Following sample collection the catheter was flushed with an equal volume of sterile saline to prevent coagulation and associated morbidity. Each blood sample was allowed to clot at room temperature for a maximum of 10 min. The coagulated blood samples were centrifuged at 14,000 rpm (approximately 5000×g) for two minutes at room temperature. A 100 μL of serum was transferred to an RIA tube for gamma counting. Any remaining serum was held at 4° C. until all the samples had been successfully analyzed. Table 18 illustrates the serum collection schedule for the study. FIG. 26 represents serum radioactivity over one week after a single injection of 1 mg/kg ¹²⁵I-Repligal®.

TABLE 18
Serum Collection Schedule

| Rat ID | Day 0 | Day 1 | Day 2 | Day 3 | Day 4 | Day 5 | Day 6 | Day 7 |
|---|---|---|---|---|---|---|---|---|
| #A1-A3 | 0.5 h | 24 h^{§} | | | | | | |
| #B1-B3 | 1 h 2 h | | | | | | | |
| | 4 h 8 h | | | | | | | |
| | 12 h | | | | | | | |
| #A4-A6 | | 24 h | 48 h^{§} | | | | | |
| #B4-B6 | | 26 h | | | | | | |
| | | 28 h | | | | | | |
| | | 30 h | | | | | | |
| | | 36 h | | | | | | |
| #A7-A9 | | 24 h | 48 h | 72^{§} | | | | |
| #B7-B9 | | | 50 h | | | | | |
| | | | 52 h | | | | | |
| | | | 54 h | | | | | |
| | | | 60 h | | | | | |
| #A10-A12 | | | 48 h | 72 h | 96 h^{§} | | | |
| #B10-B12 | | | | 74 h | | | | |
| | | | | 76 h | | | | |
| | | | | 78 h | | | | |
| | | | | 84 h | | | | |
| #A13-A15 | | | | 72 h | 96 h | 120 h^{§} | | |
| #B13-B15 | | | | | 98 h | | | |
| | | | | | 100 h | | | |
| | | | | | 104 h | | | |
| | | | | | 108 h | | | |
| #A16-A18 | | | | | 96 h | 120 h | 144 h^{§} | |
| #B16-B18 | | | | | | 122 h | | |
| | | | | | | 124 h | | |
| | | | | | | 128 h | | |
| | | | | | | 132 h | | |
| #A19-A21 | | | | | | 120 h | 144 h | 168 h^{§} |
| #B19-B21 | | | | | | | 146 h | |
| | | | | | | | 148 h | |
| | | | | | | | 152 h | |
| | | | | | | | 156 h | |

^{§}indicates terminal blood sampling

### Biodistribution in Target Tissues

In order to assess the biodistribution of ¹²⁵I-Replagal® of the study duration groups of n=3 rats were sacrificed from either group every 24 hr following treatment. The liver, kidney, heart, spleen, injection site, and thyroid were harvested for analysis. Each tissue sample was harvested by blunt dissection and placed immediately in a 5-ml plastic conical tube (Fisher Scientific, Chicago, Ill.). The thyroid was harvested to assess uptake of radiolabelled iodine from systemic circulation not as a target tissue for Replagal®. Immediately after harvest, the entire organ (or pooled organs in the case of the kidneys and testes) was weighed; these values were recorded as “total organ weight (g).” Table 19 illustrates the tissue collection schedule for the study.

TABLE 19
Tissue collection schedule post-injection.

| | Hours | | | | | | |
|---|---|---|---|---|---|---|---|
| | 24 h | 48 h | 72 h | 96 h | 120 h | 144 h | 168 h |
| | Days | | | | | | |
| | 1 | 2 | 3 | 4 | 5 | 6 | 7 |
| Rat | #A1-A3 | #A4-A6 | #A7-A9 | #A10-A12 | #A13-A15 | #A16-A18 | #A19-A21 |
| ID | #B1-B3 | #B4-B6 | #B7-B9 | #B10-B12 | #B13-B15 | #B16-B18 | #B19-B21 |

### Gamma Counting

The radioactivity of tissue samples from rats injected with ¹²⁵I-Replagal® was quantified using a Wallace WIZARD automatic gamma counter (PerkinElmer, Boston, Mass.). Tissue samples were loaded into 12×55 mm polycarbonate RIA (radioimmunoassay) tubes (PerkinElmer, Boston, Mass.). A pre-programmed protocol was utilized for the analysis. Briefly, the disintegrations per minute (DPM) of each sample were measured over 60 sec. The DPM was then converted to CPM using an internal efficiency algorithm.

### Data Analysis

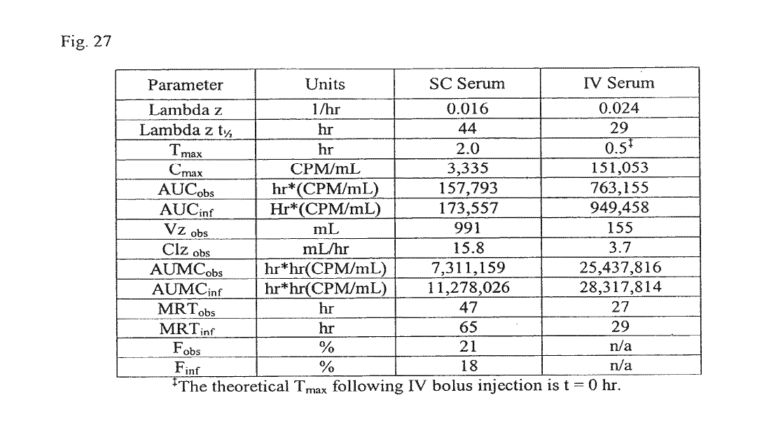

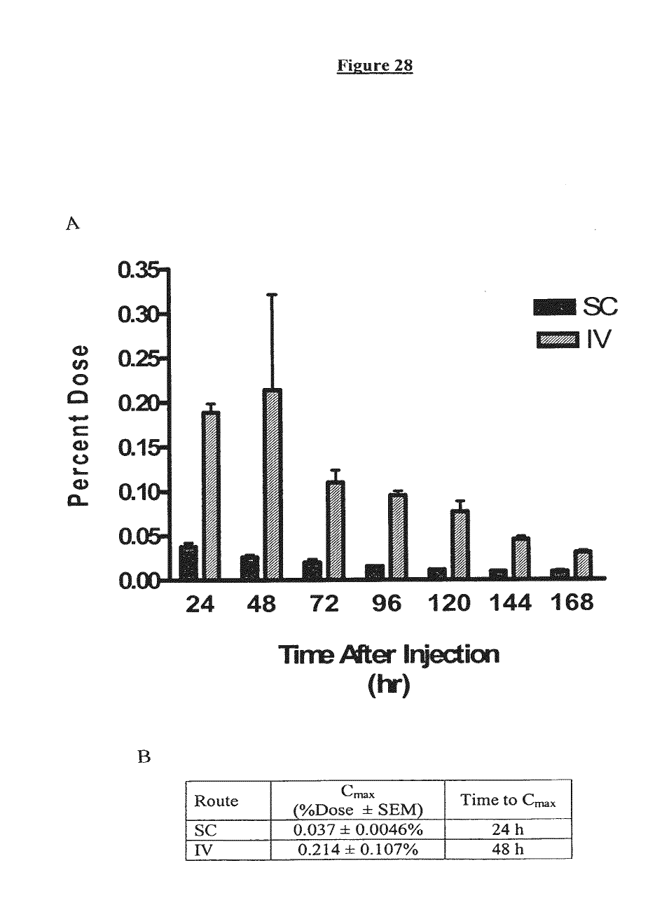

Serum CPM/mL values were calculated sample CPM and the known sample volume (0.1 mL). Total organ CPM was based on the relationship=(Sample CPM/Sample weight in g)*(Organ weight in g). Percent dose in tissue was calculated by dividing the total organ CPM by the dose (CPM) administered to each mouse. Non-compartmental serum pharmacokinetic parameters were calculated using WinNonLin Professional, version 5.0.1 (Pharsight, Mountain View, Calif.). The best-fit lambda z curve was selected based on correlation values of R²=0.90 or greater. WNL calculated several key parameters, including: maximal serum radioactivity (Cₘₐₓ), area under the curve extrapolated to infinity (AUC_{inf}), predicted volume of distribution (Vd_{pred}), predicted total clearance (Cl_{pred}), and mean residence time extrapolated to infinity (MRT_{inf}). Fraction available in serum was calculated manually using the relationship, F(%)=[(AUCᵢᵥ)/AUC_{sc})]*100. Tissue half-life was calculated from the best-fit lambda z line against log -transformed total organ CPM vs. time curves using the relationship t½=(ln 2)/(-slope). Serum and tissue radioactivity-time curves were created in GraphPad Prism v.4.0 software (San Diego, Calif.) and Microsoft Excel (v. 2003, Redmond, Wash.). A summary of WinNonLin NCA results for serum radioactivity after a single 1 mg/kg injection of ¹²⁵I-Replagal® is presented in FIG. 27. FIG. 28 shows results from a single injection of 1 mg/kg ¹²⁵I-Replagal® in rat kidney, including data on time to maximal serum CPM/mL (Cₘₐₓ) following injection.

### DISCUSSION Summary of Key Points:

A single injection of SC Replagal® exhibited compartmental pharmacokinetics over the duration of this study. The terminal phase predicted the majority of serum PK, with linear elimination beginning several hours after injection (FIGS. 26 and 27).
The elimination half-life for SC Replagal® was calculated at 44 hr, compared to 29 hr for test article injected IV.
The fraction available (“bioavailability”) of SC Replagal® versus IV Replagal® was 18-21% depending on the AUC calculation method.
Rats treated with SC Replagal® had lower liver radioactivity than animals injected intravenously.
Kidney levels were comparable between groups, with IV-treated animals exhibited higher overall radioactivity compared to rats injected SC.
The tissue half-life for kidneys from SC-treated rats was increased 130% compared to IV-treated animals.

### Equivalents

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference herein in their entirety.

What is claimed is:

1. A composition comprising from about 1 mg/ml to about 60 mg/ml alpha-galactosidase A (α-Gal from about 2% to about 10% (w/v) carbohydrate, from about 5 mM to about 10 mM citrate, up to 3% (v/v) excipient, from about 0.05% to about 0.5% (v/v) surfactant, and having a pH of 6.0.

2. The composition of claim 1, wherein the carbohydrate is sucrose.

3. The composition of claim 1, wherein the excipient is glycerol.

4. The composition of claim 1, wherein the surfactant is poloxamer 188.

5-10. (canceled)

11. A method of enhancing delivery of alpha-galactosidase A (α-Gal A) to the kidneys in an individual with Fabry disease, the method comprising administering human α-Gal A subcutaneously or by an oral route or by a parenteral route to the individual.

12. The method of claim 11, wherein the parenteral route is selected from the group consisting of the following routes: intra-arterial, intraperitoneal, ophthalmic, intramuscular, vaginal, intraorbital, intracerebral, intradermal, intracranial, intraspinal, intraventricular, intrathecal, intracisternal, intracapsular, intrapulmonary, intranasal, transmucosal, transdermal and inhalation.

13-14. (canceled)

15. The method of claim 11, wherein alpha-galactosidase A (α-Ga A) is administered in sufficient dose to result in kidney α-Gal A levels in the individual that result in an increase in the fraction of normal glomeruli and/or a decrease in the fraction of glomeruli with mesangial widening.

16. The method of claim 11, wherein alpha-galactosidase A (α-Gal A) is isolated, genetically engineered α-Gal A.

17. (canceled)

18. The method of claim 11, wherein the alpha-galactosidase A (α-Gal A) is administered in an α-Gal A formulation.

19. (canceled)

20. The method of claim 18, wherein the formulation of the alpha-galactosidase A (α-Gal A) comprises from about 1 mg/ml to about 60 mg/ml α-Gal A, from about 2% to about 10% (w/v) carbohydrate, from about 5 mM to about 10 mM citrate, up to 3% (v/v) excipient, and from about 0.05% to about 0.5% (v/v) surfactant.

21-23. (canceled)

24. The method of claim 18, wherein the formulation comprises 30 mg/ml of alpha-galactosidase A (α-Gal A), 5% (w/v) sucrose, 5 mM citrate, between about 1% and 2.5% (v/v) glycerol, and 0.05% (v/v) poloxamer 188, and wherein the pH of the formulation is 6.0.

25. The method of claim 18, wherein the formulation of the alpha-galactosidase A (α-Gal A) is a multi-dose formulation.

26. The method of claim 18, wherein the formulation of the alpha-galactosidase A (α-Gal A) comprises from about 1 mg/ml to about 60 mg/ml α-Gal A, from about 2% to about 10% (w/v) carbohydrate, from about 5 mM to about 10 mM citrate, about 1% or less of an antimicrobial agent, and up to 3% (v/v) excipient.

27-28. (canceled)

29. The method of claim 26, wherein the antimicrobial agent is phenol, m-crescol, parabens, or benzyl alcohol.

30. The method of claim 25, wherein the multi-dose formulation comprises 30mWm1 of alpha-galactosidase A (α-Gal A), 5% (w/v) sucrose, 5 mM citrate, 1% or less (v/v) benzyl alcohol, up to 3% (v/v) glycerol, and has a pH of 6.0.

31. The method of any one of claims 11, wherein alpha-galactosidase A (α-Gal A) is administered once per day, once every two days, once every three days, once every four days, once every five days, or once every six days, in a dose of from about 0.1 mg to about 20 mg of α-Gal A per kg body weight.

32. The method of claim 18, wherein the alpha-galactosidase A (α-Gal A) formulation is a Replagal ® or Fabrazyme® formulation.

33. A method of producing therapeutically effective kidney levels of alpha-galactosidase A (α-Gal A) in an individual with Fabry disease, the method comprising administering subcutaneously or by an oral route or by a parenteral route to the individual a dose of from about 0.1 mg to about 20 mg of α-Gal A per kg. body weight, wherein the dose is administered once per day, once every two days, once every three days, once every four days, once every five days, or once every six days, wherein the parenteral route is selected from the group consisting of the following routes: intra-arterial, intraperitoneal, ophthalmic intramuscular, vaginal, intraorbital, intracerebral, intradermal, intracranial, intraspinal, intraventricular, intrathecal, intracisternal, intracapsular, intrapulmonary, intranasal, transmucosal, transdermal and inhalation.

34-37. (canceled)

38. The method of claim33any one of claims 33 to 37, wherein alpha-galactosidase A (α-Gal A) is isolated, genetically engineered α-Gal A.

39. (canceled)

40. The method of claim 33, wherein the alpha-galactosidase A (α-Gal A) is administered in an α-Gal A formulation.

41-75. (canceled)
